(19) United States
(12) Patent Application Publication
(10) Pub. No.: US 2026/0146264 A1
(43) Pub. Date: May 28, 2026

(54) METHODS FOR REPROGRAMMING AND GENE EDITING CELLS

(71) Applicant: Factor Bioscience Inc., Cambridge, MA (US)

(72) Inventors: Matthew ANGEL, Cambridge, MA (US); Christopher ROHDE, Cambridge, MA (US); Ismet Caglar TANRIKULU, Cambridge, MA (US); Tae Yun KIM, Cambridge, MA (US); Claire AIBEL, Cambridge, MA (US)

(21) Appl. No.: 19/374,153

(22) Filed: Oct. 30, 2025

Related U.S. Application Data
(63) Continuation of application No. PCT/US2024/027247, filed on May 1, 2024.
(60) Provisional application No. 63/463,295, filed on May 1, 2023.

(51) Int. Cl. C12N 15/90; C12N 9/16; C12N 9/22; C12N 15/11

(52) U.S. Cl. CPC C12N 15/907; C12N 9/16; C12N 9/224; C12N 15/11; C12Y 301/21004; C12N 2310/20; C12N 2310/315; C12N 2820/002

(57) ABSTRACT

The present disclosure provides improved methods for reprogramming and gene editing cells. In one aspect, the present disclosure provides a method of introducing a single strand break in a DNA site. In one aspect, the present disclosure provides a method of inserting a sequence in a DNA site by introducing a single strand break followed by insertion of the sequence using a single-stranded repair template. In another aspect, the present disclosure provides a method of activating gene expression in a cell during its differentiation. In another aspect, the present disclosure methods and compositions related to iPSC-derived mesenchymal stroma/stem cells that overexpressed IDO1. In another aspect, the present disclosure provides a solid support comprising iPSC-derived mesenchymal stem cells which can be used to treat organ damage.

### CROSS-REFERENCE

This application is a continuation of International Application No. PCT/US2024/027247 filed May 1, 2024, which claims the benefit to U.S. Provisional Application No. 63/463,295 filed May 1, 2023, each of which is incorporated herein by reference in its entirety.

### BACKGROUND

Methods for reprogramming differentiated cells into pluripotent cells and methods for gene-editing cells have progressed greatly over recent years. However, there remain unmet needs for improved methods for reprogramming and gene editing cells.

Gene-editing nickases create targeted single-strand breaks (SSBs) that favor high-fidelity repair through the homology-directed repair (HDR) pathway rather than the more error-prone non-homologous end joining (NHEJ) or microhomology-mediated end joining (MMEJ) pathways. However, there remain needs for improved methods and compositions for gene editing via single-stranded breaks.

Currently, when skilled artisans want to activate genes during cell culturing, they add components (e.g., cytokines) to a culturing medium. Improved methods and compositions for controlling gene activation during cell during, particularly before, during, and/or cell differentiation, are needed.

The many known drawbacks of RNA-based reprogramming methods make them undesirable for research, therapeutic or cosmetic use. There is an unmet need for improved RNA-based reprogramming methods.

Compared with double-stranded DNA (dsDNA), single-stranded DNA (ssDNA) exhibits lower toxicity and is less prone to random genomic integration, making it a suitable form of DNA donor for gene knock-in. However, current methods of synthesizing ssDNA, including enzymatic digestion, asymmetric PCR, and chemical synthesis, suffer from low yields, contamination with residual dsDNA, length limitations, and high cost. There remain needs for improved method of producing ssDNA donor.

### SEQUENCE LISTING

The instant application contains a Sequence Listing which has been submitted electronically in XML format and is hereby incorporated by reference in its entirety. The XML copy, created on Oct. 29, 2025, is named 61057-721301.xml and is 132,350 bytes in size.

### SUMMARY

Provided herein is a method for creating a single-stranded break in a DNA site, the method comprising: (a) contacting the DNA with a first gene-editing protein comprising a nuclease domain (1) capable of binding to the DNA and forming a dimer with another nuclease domain, and (2) capable of creating a single-stranded break in a DNA site; and (b) contacting the DNA with a second gene-editing protein comprising a nuclease domain (1) capable of binding to the DNA and forming a dimer with another nuclease domain, and (2) incapable of creating a single-stranded break in a DNA site.

In some embodiments, the nuclease domain incapable of creating a single-stranded break in a DNA site comprises a mutation in its catalytic domain.

In some embodiments, the method further comprises contacting the DNA with a single-stranded repair template.

In some embodiments, the single-stranded repair template integrates into the DNA at the single-stranded break.

Also provided herein is a method for creating a single-stranded break in a DNA site of a cell, the method comprising contacting the cell with a pair of synthetic RNAs comprising a first synthetic RNA and a second synthetic RNA, wherein the first synthetic RNA encodes a gene-editing protein comprising a nuclease domain (1) capable of binding to the DNA and forming a dimer with another nuclease domain, and (2) capable of creating a single-stranded break in a DNA site of the cell, and wherein a second synthetic RNA encoding a gene-editing protein comprising a nuclease domain (1) capable of binding to the DNA and forming a dimer with another nuclease domain, and (2) incapable of creating a single-stranded break in a DNA site of the cell.

In some embodiments, the nuclease domain incapable of creating a single-stranded break in a DNA site comprises a mutation in its catalytic domain.

In some embodiments, the method further comprises contacting the cell with a single-stranded repair template.

In some embodiments, the single-stranded repair template is DNA.

In some embodiments, the single-stranded repair template integrates into the DNA of the cell at the single-stranded break.

In some embodiments, the nuclease domain incapable of creating a single-stranded break in a DNA site comprises a FokI domain.

In some embodiments, the FokI domain bears a mutation.

In some embodiments, the mutation is selected from the group consisting of D67A, D67N, D84A, and any combinations thereof, each of which is numbered in reference to the sequence of SEQ ID NO: 80.

In some embodiments, the mutation is a D67A mutation and the FokI domain comprises a sequence with at least 80%, 85%, 90%, 95%, 96%, 98%, or 99% sequence identity to SEQ ID NO: 81.

In some embodiments, the FokI domain is encoded by a nucleic acid comprising a sequence with at least 80%, 85%, 90%, 95%, 96%, 98%, or 99% sequence identity to SEQ ID NO: 85.

In some embodiments, the mutation is a D67N mutation and the FokI domain comprises a sequence with at least 80%, 85%, 90%, 95%, 96%, 98%, or 99% sequence identity to SEQ ID NO: 82.

In some embodiments, the FokI domain is encoded by a nucleic acid comprising a sequence with at least 80%, 85%, 90%, 95%, 96%, 98%, or 99%, or 100% sequence identity to SEQ ID NO: 86.

In some embodiments, the mutation is a D67A mutation and the FokI domain comprises a sequence with at least 80%, 85%, 90%, 95%, 96%, 98%, or 99%, or 100% sequence identity to SEQ ID NO: 83.

In some embodiments, the FokI domain is encoded by a nucleic acid comprising a sequence with at least 80%, 85%, 90%, 95%, 96%, 98%, or 99%, or 100% sequence identity to SEQ ID NO: 87.

In some embodiments, the first gene editing protein and/or the second gene editing protein comprises a plurality of repeat sequences and at least one of the repeat sequences comprises the amino acid sequence: LTPvQVVAIAwxyz (SEQ ID NO: 16), wherein: (a) v is Q, D or E, (b) w is S or N, (c) x is I, H, or N, (d) y is D, A, I, N, H, K, S, G or null, and (e) z is

```
  (SEQ ID NO: 23)
GGRPALE,
(SEQ ID NO: 24)
GGKQALE,
(SEQ ID NO: 25)
GGKQALETVQRLLPVLCQDHG,
(SEQ ID NO: 26)
GGKQALETVQRLLPVLCQAHG,
  (SEQ ID NO: 27)
GKQALETVQRLLPVLCQDHG,
  (SEQ ID NO: 28)
GKQALETVQRLLPVLCQAHG,
  (SEQ ID NO: 19)
GGKQALETVQRLLPVLCQD
or
  (SEQID NO: 20)
GGKQALETVQRLLPVLCQA.
```

In some embodiments, the first gene editing protein and/or the second gene editing protein comprises a plurality of repeat sequences and at least one of the repeat sequences comprises the amino acid sequence: LTPvQVVAIAwxyzα (SEQ ID NO: 22), wherein: (a) v is Q, D or E, (b) w is S or N, (c) x is I, H, or N, (d) y is D, A, I, N, H, K, S, G or null, (e) z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), and (f) α is any four consecutive amino acids.

In some embodiments, α comprises at least one glycine (G) residue.

In some embodiments, α comprises at least one histidine (H) residue.

In some embodiments, α comprises at least one histidine (H) residue at any one of positions 33, 34, or 35.

In some embodiments, α comprises at least one aspartic acid (D) residue.

In some embodiments, α comprises at least one, or two, or three of a glycine (G) residue, a histidine (H) residue, and an aspartic acid (D) residue.

In some embodiments, α comprises one or more hydrophilic residues, optionally selected from: (a) a polar and positively charged hydrophilic amino acid, optionally selected from arginine (R) and lysine (K); (b) a polar and neutral of charge hydrophilic amino acid, optionally selected from asparagine (N), glutamine (Q), serine (S), threonine (T), proline (P), and cysteine (C); (c) a polar and negatively charged hydrophilic amino acid, optionally selected from aspartate (D) and glutamate (E), and (d) an aromatic, polar and positively charged hydrophilic amino acid, optionally selected from histidine (H).

In some embodiments, α comprises one or more hydrophobic residues, optionally selected from: (a) a hydrophobic, aliphatic amino acid, optionally selected from glycine (G), alanine (A), leucine (L), isoleucine (I), methionine (M), and valine (V), and (b) a hydrophobic, aromatic amino acid, optionally selected from phenylalanine (F), tryptophan (W), and tyrosine (Y).

In some embodiments, u is selected from GHGG (SEQ ID NO: 31), HGSG (SEQ ID NO: 32), HGGG (SEQ ID NO: 33), GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60).

In some embodiments, the first gene editing protein and/or the second gene editing protein comprises: (a) a plurality of repeat sequences and at least one of the repeat sequences comprises the amino acid sequence: LTPvQVVAIAwxyzGHGG (SEQ ID NO: 17), wherein: “v” is Q, D or E, “w” is S or N, “x” is H, N, or I, “y” is D, A, I, N, G, H, K, S, G or null, and “z” is GGKQALETVQRLLPVLCQD (SEQ ID NO: 19) or GGKQALETVQRLLPVLCQA (SEQ ID NO: 20); and (b) a nuclease domain comprising a catalytic domain of a nuclease.

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is any amino acid other than N, H and I, “y” is any amino acid or no amino acid, and “z” is

```
  (SEQ ID NO: 23)
GGRPALE,
  (SEQ ID NO: 24)
GGKQALE,
  (SEQ ID NO: 19)
GGKQALETVQRLLPVLCQD,
  (SEQ ID NO: 20)
GGKQALETVQRLLPVLCQA,
  (SEQ ID NO: 69)
GKQALETVQRLLPVLCQD
or
(SEQ ID NO: 68)
GKQALETVQRLLPVLCQA.
```

In some embodiments, the first gene editing protein and/or the second gene editing protein comprises a plurality of repeat sequences and at least one of the repeat sequences comprises the amino acid sequence: LTPvQVVAIAwIyzGHGG (SEQ ID NO: 88), wherein “v” is Q, D or E, “w” is S or N, “y” is any amino acid other than G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwIAzGHGG (SEQ ID NO: 71), wherein “v” is Q, D or E, “w” is S or N, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwx (SEQ ID NO: 67), wherein “v” is Q, D or E, “w” is S or N, and “x” is S, T or Q.

In some embodiments, the first gene editing protein and/or the second gene editing protein comprises a plurality of repeat sequences and at least one of the repeat sequences comprises the amino acid sequence: LTPvQVVAIAwxy (SEQ ID NO: 72), wherein “v” is Q, D or E, “w” is S or N, “x” is S, T or Q, and “y” is selected from: D, A, I, N, H, K, S, and G.

Also provided herein is a pair of synthetic RNAs comprising a first synthetic RNA and a second synthetic RNA, wherein the first synthetic RNA encodes a gene-editing protein comprising a nuclease domain (1) capable of binding to the DNA and forming a dimer with another nuclease domain, and (2) capable of creating a single-stranded break in a DNA site, and wherein the second synthetic RNA encodes a gene-editing protein comprising a nuclease domain (1) capable of binding to the DNA and forming a dimer with another nuclease domain, and (2) incapable of creating a single-stranded break in a DNA site.

In some embodiments, the nuclease domain incapable of creating a single-stranded break in a DNA site comprises a FokI domain.

In some embodiments, the FokI domain bears a mutation.

In some embodiments, the mutation is selected from the group consisting of D67A, D67N, D84A, and any combinations thereof, each of which is numbered in reference to the sequence of SEQ ID NO: 80.

In some embodiments, the mutation is a D67A mutation and the FokI domain comprises a sequence with at least 80%, 85%, 90%, 95%, 96%, 98%, or 99% sequence identity to SEQ ID NO: 81.

In some embodiments, the FokI domain is encoded by a nucleic acid comprising a sequence with at least 80%, 85%, 90%, 95%, 96%, 98%, or 99% sequence identity to SEQ ID NO: 85.

In some embodiments, the mutation is a D67N mutation and the FokI domain comprises a sequence with at least 80%, 85%, 90%, 95%, 96%, 98%, or 99% sequence identity to SEQ ID NO: 82.

In some embodiments, the FokI domain is encoded by a nucleic acid comprising a sequence with at least 80%, 85%, 90%, 95%, 96%, 98%, or 99%, or 100% sequence identity to SEQ ID NO: 86.

In some embodiments, the mutation is a D67A mutation and the FokI domain comprises a sequence with at least 80%, 85%, 90%, 95%, 96%, 98%, or 99%, or 100% sequence identity to SEQ ID NO: 83.

In some embodiments, the FokI domain is encoded by a nucleic acid comprising a sequence with at least 80%, 85%, 90%, 95%, 96%, 98%, or 99%, or 100% sequence identity to SEQ ID NO: 87.

In some embodiments, the first gene editing protein and/or the second gene editing protein comprises a plurality of repeat sequences and at least one of the repeat sequences comprises the amino acid sequence: LTPvQVVAIAwxyz (SEQ ID NO: 16), wherein: (a) v is Q, D or E, (b) w is S or N, (c) x is I, H, or N, (d) y is D, A, I, N, H, K, S, G or null, and (e) z is

```
  (SEQ ID NO: 23)
GGRPALE,
  (SEQ ID NO: 24)
GGKQALE,
(SEQ ID NO: 25)
GGKQALETVQRLLPVLCQDHG,
(SEQ ID NO: 26)
GGKQALETVQRLLPVLCQAHG,
  (SEQ ID NO: 27)
GKQALETVQRLLPVLCQDHG,
  (SEQ ID NO: 28)
GKQALETVQRLLPVLCQAHG,
  (SEQ ID NO: 29)
GGKQALETVQRLLPVLCQD
or
  (SEQ ID NO: 20)
GGKQALETVQRLLPVLCQA.
```

In some embodiments, the first gene editing protein and/or the second gene editing protein comprises a plurality of repeat sequences and at least one of the repeat sequences comprises the amino acid sequence: LTPvQVVAIAwxyzα (SEQ ID NO: 22), wherein: (a) v is Q, D or E, (b) w is S or N, (c) x is I, H, or N, (d) y is D, A, I, N, H, K, S, G or null, (e) z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), and (f) α is any four consecutive amino acids.

In some embodiments, α comprises at least one glycine (G) residue.

In some embodiments, α comprises at least one histidine (H) residue.

In some embodiments, α comprises at least one histidine (H) residue at any one of positions 33, 34, or 35.

In some embodiments, α comprises at least one aspartic acid (D) residue.

In some embodiments, α comprises at least one, or two, or three of a glycine (G) residue, a histidine (H) residue, and an aspartic acid (D) residue.

In some embodiments, α comprises one or more hydrophilic residues, optionally selected from: (a) a polar and positively charged hydrophilic amino acid, optionally selected from arginine (R) and lysine (K); (b) a polar and neutral of charge hydrophilic amino acid, optionally selected from asparagine (N), glutamine (Q), serine (S), threonine (T), proline (P), and cysteine (C); (c) a polar and negatively charged hydrophilic amino acid, optionally selected from aspartate (D) and glutamate (E), and (d) an aromatic, polar and positively charged hydrophilic amino acid, optionally selected from histidine (H).

In some embodiments, α comprises one or more hydrophobic residues, optionally selected from: (a) a hydrophobic, aliphatic amino acid, optionally selected from glycine (G), alanine (A), leucine (L), isoleucine (I), methionine (M), and valine (V), and (b) a hydrophobic, aromatic amino acid, optionally selected from phenylalanine (F), tryptophan (W), and tyrosine (Y).

In some embodiments, a is selected from GHGG (SEQ ID NO: 31), HGSG (SEQ ID NO: 32), HGGG (SEQ ID NO: 33), GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60).

In some embodiments, the first gene editing protein and/or the second gene editing protein comprises: (a) a plurality of repeat sequences and at least one of the repeat sequences comprises the amino acid sequence: LTPvQVVAIAwxyzGHGG (SEQ ID NO: 17), wherein: “v” is Q, D or E, “w” is S or N, “x” is H, N, or I, “y” is D, A, I, N, G, H, K, S, G or null, and “z” is GGKQALETVQRLLPVLCQD (SEQ ID NO: 19) or GGKQALETVQRLLPVLCQA (SEQ ID NO: 20); and (b) a nuclease domain comprising a catalytic domain of a nuclease.

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is any amino acid other than N, H and I, “y” is any amino acid or no amino acid, and “z” is

```
  (SEQ ID NO: 23)
GGRPALE,
  (SEQ ID NO: 24)
GGKQALE,
  (SEQ ID NO: 19)
GGKQALETVQRLLPVLCQD,
  (SEQ ID NO: 20)
GGKQALETVQRLLPVLCQA,
(SEQ ID NO: 69)
GKQALETVQRLLPVLCQD
or
(SEQ ID NO: 68)
GKQALETVQRLLPVLCQA
```

In some embodiments, the first gene editing protein and/or the second gene editing protein comprises a plurality of repeat sequences and at least one of the repeat sequences comprises the amino acid sequence: LTPvQVVAIAwIyzGHGG (SEQ ID NO: 88), wherein “v” is Q, D or E, “w” is S or N, “y” is any amino acid other than G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwIAzGHGG (SEQ ID NO: 71), wherein “v” is Q, D or E, “w” is S or N, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwx (SEQ ID NO: 67), wherein “v” is Q, D or E, “w” is S or N, and “x” is S, T or Q.

In some embodiments, the first gene editing protein and/or the second gene editing protein comprises a plurality of repeat sequences and at least one of the repeat sequences comprises the amino acid sequence: LTPvQVVAIAwxy (SEQ ID NO: 72), wherein “v” is Q, D or E, “w” is S or N, “x” is S, T or Q, and “y” is selected from: D, A, I, N, H, K, S, and G.

Also provided herein is a composition comprising the pair of synthetic RNAs of any one of the foregoing embodiments.

In some embodiments, the composition further comprises a single-stranded DNA template.

Also provided herein is a kit comprising the pair of synthetic RNAs of any one of ethe foregoing embodiments and a single-stranded DNA template.

Also provided herein is a method for activating gene expression, the method comprising: obtaining a cell comprising a transgene which expresses a protein of interest under the control of a promoter which is active during a defined period of a cell's differentiation and, when the cell's differentiation state is before or after the defined period during which the promoter is active, contacting the cell with a compound that demethylates DNA.

In some embodiments, the promoter has reduced activity as the cell differentiates, and wherein the method comprises contacting the cell with the compound that demethylates DNA during differentiation to reactivate the promoter.

In some embodiments, the promoter is inactive once the cell has differentiated, and wherein the method comprises contacting the cell with the compound that demethylates DNA after the cell has differentiated to reactivate the promoter.

In some embodiments, the promoter is active as the cell differentiates, and wherein the method comprises contacting the cell with the compound that demethylates DNA before the cell begins to differentiate to activate the promoter.

In some embodiments, the compound that demethylates DNA is a selective histone deacetylase (HDAC) inhibitor.

In some embodiments, wherein the selective HDAC inhibitor is selected from the group consisting of: trapoxin, suberanilohydroxamic acid, valproate, and romidepsin, and combinations thereof, optionally the selective HDAC inhibitor is Trichostatin A or a functional derivative thereof.

In some embodiments, activating or reactivating the promoter provides expression of protein of interest at an atypical period in the cell's differentiation.

In some embodiments, expression of the protein of interest at the atypical period in the cell's differentiation changes fate of the cell and/or changes activity of the cell.

In some embodiments, changing the fate of the cell comprising differentiating the cell into a cell type or cell subtype for which it was not previously destined.

In some embodiments, the cell is an induced pluripotent stem cell (iPSC) which has not begun differentiating into an induced mesenchymal stem cell (iMSC).

In some embodiments, the cell is an induced pluripotent stem cell (iPSC) which is differentiating into an induced mesenchymal stem cell (iMSC).

In some embodiments, the promoter is a JeT promoter.

Also provided herein is a method for producing single-stranded DNA, the method comprising: (a) obtaining a double-stranded DNA, which comprises a strand comprising at least one phosphorothioate-containing nucleotide and a strand lacking a phosphorothioate-containing nucleotide, and (b) contacting the double-stranded DNA with one or more nucleases which digest the strand lacking the phosphorothioate-containing nucleotide, thereby obtaining a single stranded DNA.

In some embodiments, the double-stranded DNA is synthesized by PCR using a standard 5′ primer lacking a phosphorothioate-containing nucleotide, and a 5′ primer comprising one or more phosphorothioate-containing nucleotides, or wherein the method further comprises synthesizing the double-stranded DNA by PCR using a standard 5′ primer lacking the phosphorothioate-containing nucleotide and a 5′ primer comprising one or more phosphorothioate-containing nucleotides.

In some embodiments, the one or more nucleases comprises lambda exonuclease or T7 exonuclease.

In some embodiments, the one or more nucleases comprises lambda exonuclease and T7 exonuclease.

In some embodiments, the DNA is contacted with lambda exonuclease before contacting with T7 exonuclease.

In some embodiments, the strand comprising at least one phosphorothioate-containing nucleotide resists digestion by the one or more nucleases.

In some embodiments, the method reduces the amount of double-stranded contamination in a product comprising the single-stranded DNA by at least 95%, e.g., at least 99%.

In some embodiments, the double stranded DNA comprises at least 50% GC base pairs.

In some embodiments, the single stranded DNA is used as a repair template in the method of any one of the foregoing embodiments.

Also provided herein is a single stranded DNA comprising at least one phosphorothioate-containing nucleotide and obtained by the method of any one of the foregoing embodiments.

Also provided herein is a method for manufacturing iPSC-derived differentiated cells that are engineered to overexpress IDO1, the method comprising: obtaining an iPSC comprising a genetic modification which results in overexpression of IDO1 in the iPSC, culturing the iPSC under conditions that promote differentiation of the iPSC, and contacting the iPSC with one or more IDO1 inhibitors.

In some embodiments, the iPSC comprising the genetic modification which results in overexpression of IDO1 in the iPSC has delayed or non-existent differentiation absent contact with the one or more IDO1 inhibitors.

In some embodiments, the iPSC comprising the genetic modification which results in overexpression of IDO1 in the iPSC has increased population doubling time absent contact with the one or more IDO1 inhibitors.

In some embodiments, the contacting the iPSC with the one or more IDO1 inhibitors permits differentiation of the iPSC and/or reduces population doubling time of the iPSC.

In some embodiments, the method comprises differentiating the iPSC into a cell of the lymphoid lineage, a cell of the myeloid lineage, a hematopoietic stem cell, or a mesenchymal stromal/stem cell (MSC).

In some embodiments, the one or more IDO1 inhibitors comprise Epacadostat and/or IDO1-IN-5.

In some embodiments, the one or more IDO1 inhibitors comprise both Epacadostat and IDO1-IN-5.

Also provided herein is a method for suppressing dysregulated immune cells while simultaneously promoting expansion of regulatory T lymphocytes and the M1 (pro-inflammatory) to M2 (anti-inflammatory) polarization of macrophages in a subject in need thereof, the method comprising administering to the subject a therapeutically effective amount of an engineered cell, wherein the engineered cell is a cell of the lymphoid lineage, a cell of the myeloid lineage, a hematopoietic stem cell, or a mesenchymal stromal/stem cell (MSC), and the engineered cell comprises a genetic modification that results in overexpression of IDO1 in the engineered cell.

In some embodiments, the engineered cell is derived from an iPSC.

Also provided herein is a pharmaceutical composition comprising a therapeutically effective amount of an engineered cell, wherein the engineered cell is a cell of the lymphoid lineage, a cell of the myeloid lineage, a hematopoietic stem cell, or a mesenchymal stromal/stem cell (MSC), and the engineered cell comprises a genetic modification that results in overexpression of IDO1 in the engineered cell.

Also provided herein is a solid support comprising a therapeutically effective amount of mesenchymal stromal/stem cells (MSCs), wherein the MSCs comprise a genetic modification resulting in overexpression of IDO1 in the MSCs, and are derived from an iPSC.

Also provided herein is a solid support comprising a therapeutically effective amount of the mesenchymal stromal/stem cells (MSCs) manufactured by the method of any one of the foregoing embodiments.

In some embodiments, the solid support comprises a patch.

In some embodiments, the solid support comprises a sheet comprising collagen.

In some embodiments, the solid support further comprises a polymer.

In some embodiments, the solid support further comprises a hydrogel.

Also provided herein is a method for treating a damaged organ, the method comprising contacting a damaged portion of the damaged organ with the solid support of any one of the foregoing embodiments.

Also provided herein is a method for treating a damaged organ, the method comprising contacting a damaged portion of the damaged organ with a solid support comprising a therapeutically effective amount of mesenchymal stromal/stem cells (MSCs), wherein the MSCs comprise a genetic modification resulting in overexpression of IDO1 in the MSCs, and are derived from an iPSC.

### BRIEF DESCRIPTION OF THE DRAWINGS

FIG. 1 is a cartoon showing that gene editing proteins of the present disclosure, which comprise one mutant nuclease domain, can enable scarless targeted gene insertion in primary human cells.

FIG. 2A is an illustration of a wild type COL7A1_e73 gene-editing protein pair bound to target DNA. FIG. 2B is an illustration of COL7A1_e73 gene editing protein of the present disclosure, which comprises one mutant nuclease domain and is bound to target DNA.

FIG. 3 shows a picture of a gel (left) and a graph (right) depicting results of primary human fibroblasts that were transfected with gene-editing protein pairs.

FIG. 4 shows a picture of a gel (left) and a graph (right) depicting results of iPSCs that were transfected with gene-editing protein pairs.

FIG. 5 shows a picture of a gel (top left) and a graph (top right) depicting results of the relative insertion efficiency of a donor sequence into primary human fibroblasts, which were transfected with gene-editing protein pairs. Also shown in the figure are DNA sequences (bottom) from primary human fibroblasts that were transfected with gene-editing protein pairs.

FIG. 6 shows a picture of a gel (left) and a graph (right) depicting results of the relative insertion efficiency of a donor sequence into iPSCs that were transfected with gene-editing protein pairs.

FIG. 7 shows a picture of a gel (left) and a graph (right) depicting results of the relative insertion efficiency of a donor sequence into human iMSCs that were transfected with gene-editing protein pairs.

FIG. 8 is a schematic showing that efficient transgene knock-in in human iPS cells combined with small molecule treatment yields clonal populations of engineered tissue-specific cells.

FIG. 9A is an image of iMSCs expressing GFP under a pJet promoter. FIG. 9B is an image of iMSCs expressing GFP under a Ef1a promoter.

FIG. 10 is an image of a gel showing pJet and EF1a GFP insertion into amplicons.

FIG. 11 is an image of a gel showing integration of a repair template in iPSCs.

FIG. 12A is an image of hiPSCs transfected with GFP under a pJet promoter. FIG. 12B is an image of hiPSCs transfected with GFP under an Ef1a promoter.

FIG. 13A is a FACS cytometry cell sorting graph of cells expressing GFP under a pJet promoter. FIG. 13B is a picture of a gel demonstrating homozygous and heterologous GFP transgene insertion in target gene region of engineered cells as compared to wildtype (uninserted) cells.

FIG. 14A is a FACS cytometry cell sorting graph of cells expressing GFP under a Ef1a promoter. FIG. 14B is a picture of a gel demonstrating homozygous and heterologous GFP transgene insertion in target gene region of engineered cells as compared to wildtype (uninserted) cells.

FIG. 15 is a flowchart of the Stemdiff™ Mesenchymal Progenitor kit protocol for differentiating iPSCs to iMSCs.

FIG. 16A shows bright field and GFP fluorescent images of cells. FIG. 16B shows GFP fluorescent images of cells treated with TSA.

FIG. 17A is bright field and GFP fluorescent images of cells. FIG. 17B is images of bulk sorted GFP fluorescent cells.

FIG. 18 is a schematic showing that gene-editing proteins can be used to create single- or double-strand breaks at specific genomic sites.

FIG. 19 is a schematic showing an overview of single-stranded DNA synthesis from plasmid DNA.

FIG. 20 is a picture of a gel showing digestion of DNA with lambda or T7 exonuclease.

FIG. 21 is a picture of a gel showing the removal of double-stranded DNA product using T7 and/or lambda exonuclease.

FIG. 22 presents pictures of a gel (left) showing undigested and digested dsDNA from samples A, B, and C, and a gel (right) showing digested samples A, B, and C that were treated with T7 exonuclease.

FIG. 23 is a picture of a gel showing a comparison between precursor dsDNA and purified ssDNA.

FIG. 24 is a picture of a gel showing a method of calculating the percentage of residual dsDNA in a ssDNA sample.

FIG. 25 presents a picture of a gel showing gene-edited ssDNA donor samples (left), and a table describing ssDNA donor size (kb) and insertion efficiency (%) (right).

FIG. 26 is a graph showing the insertion efficiency for ssDNA donors of varying sizes.

FIG. 27 shows a bright field and a GFP fluorescent image of transfected iPS cells.

FIG. 28 is a schematic showing methods for inhibiting a transgene-encoded protein.

FIG. 29 is a flow chart showing the process of gene edited iPS cell line development.

FIG. 30 is a schematic showing an IDO1-encoding single-stranded repair template.

FIG. 31 is a picture of a gel showing control and IDO1-inserted iPSCs.

FIG. 32 shows a schematic of an iPSC differentiation protocol (top), and images of iPSCs (bottom).

FIG. 33 shows flow cytometry histograms.

FIG. 34 shows a graph of iMSC doubling time.

FIG. 35 shows a graph showing relative viability of iMSCs.

### DETAILED DESCRIPTION

Gene Editing Proteins with Nickase Functionality

In some aspects, the present disclosure provides novel gene editing proteins which create single-stranded breaks in a target DNA site.

In some aspects, here are described uses of gene-editing proteins of the present disclosure, which comprise one mutant nuclease domain containing cleavage domain variants with nickase functionality for targeted insertion of donor sequences into a defined genomic locus.

In some cases, gene-editing proteins comprising a FokI catalytic domain require dimerization by two catalytic domains to create a break in a DNA sequence. The mutations disclosed herein can permit dimerization of two catalytic domains, however, the mutations prevent catalytic activity by FokI catalytic domain comprising the mutation but does not affect the ability of the wild-type FokI catalytic domain to catalyze a break in the DNA sequence. Together, by including the mutant FokI catalytic domain, rather than enacting a double-strand break in the DNA, which would occur when both domains lack mutations, the gene-editing proteins of this aspect can enact only a single stranded break.

In some cases, this feature provides a surprising benefit when gene editing cells. Following a double-stranded break, often there is some “chew back” which can introduce undesired deletions in the genomic DNA and leave a “scar” in the genome. Also, it is known that the NHEJ and MMEJ pathways can further introduce deletions and “scars”. Further, when a gene-editing protein binds and cleaves an off-target location, this can introduce an unwanted mutation, which may lead to cell death or a cancerous transformation; whereas, using gene-editing proteins of this aspect, if there is a single-stranded off-target cleavage, the cell heals single stranded break and without introducing a mutation.

On the other hand, when the gene-editing proteins of the present disclosure are combined with a single-stranded repair template, e.g., a Homology-directed repair (HDR) template, the repair template is accurately and efficiently inserted into the single-strand break. The efficiency of insertion is particularly surprising. Like other well-known gene-editing proteins, e.g., TALEN and ZFNs, a skilled artisan would expect that insertion of a repair template would require a double-stranded break and a double-stranded repair template. However, efficiency using the gene-editing proteins of this aspect are comparable if not better than efficiency using editing requiring double-stranded breaks.

Notably, use of a double-stranded repair templates often can result in off-target insertions, which again may lead to cell death or a cancerous transformation; however, the cell's natural repair machinery does not readily recognize the single-stranded repair template and will not integrate the template into the genome; thereby, the risk of mutations can be lessened with creating only a single-strand break. Additionally, if a double-stranded repair template is inserted into an undesired locus in the genome, the cell may express the protein the template codes for (e.g., a selectable marker or GFP), as a marker of a positive/accurate insertion; however, its insertion may be in the wrong locus, and this would require sequencing of the insert and its surroundings to verify accuracy of insertion. In contrast, expression of the single-stranded repair template would not occur absent insertion at the desired location.

Further, one would expect that the cell's natural repair machinery would identify single strand breaks and repair them immediately. However, the data generated by exemplary gene-editing proteins according to some embodiments of this aspect of the present disclosure suggest that the gene-editing proteins can create single-strand break that remains in place until its repair template can be inserted. Notably, if the natural repair machinery repairs the single-stranded break before insertion of the repair template, in some cases, the gene-editing protein of the present disclosure is capable of re-cleaving the target DNA and creating a repeat single-stranded break such that another opportunity arises to insert the single-stranded repair template before the cell's natural repair machinery has another opportunity to act.

In some cases, the gene-editing proteins of the present disclosure provide surprisingly efficient and accurate single-stranded breaks which do not introduce unwanted mutations and have a reduced chance of off-target mutations.

An aspect of the present disclosure is a method for creating a single-stranded break in a DNA site. The method comprises (a) contacting the DNA with a first gene-editing protein comprising a nuclease domain (1) capable of binding to the DNA and forming a dimer with another nuclease domain and (2) capable of creating a single-stranded break in a DNA site; and (b) contacting the DNA with a second gene-editing protein comprising a nuclease domain (1) capable of binding to the DNA and forming a dimer with another nuclease domain and (2) incapable of creating a single-stranded break in a DNA site.

In embodiments, the nuclease domain incapable of creating a single-stranded break in a DNA site comprises a mutation in its catalytic domain. In some cases, the method further comprises contacting the DNA with a single-stranded repair template, which sequence can be integrated into the DNA at the single-stranded break.

Another aspect of the present disclosure is a method for creating a single-stranded break in a DNA site of a cell. In some cases, the method comprises contacting the cell with a pair of synthetic RNAs, comprising a first synthetic RNA and a second synthetic RNA, wherein the first synthetic RNA encodes a gene-editing protein comprising a nuclease domain (1) capable of binding to the DNA and forming a dimer with another nuclease domain, and (2) capable of creating a single-stranded break in a DNA site of the cell, and wherein a second synthetic RNA encoding a gene-editing protein comprising a nuclease domain (1) capable of binding to the DNA and forming a dimer with another nuclease domain, and (2) incapable of creating a single-stranded break in a DNA site of the cell.

In embodiments, the nuclease domain incapable of creating a single-stranded break in a DNA site comprises a mutation in its catalytic domain. In some cases, the method further comprises contacting the cell with a single-stranded repair template, e.g., which sequence can be integrated into the DNA of the cell at the single-stranded break.

A further aspect of the present disclosure is a pair of synthetic RNAs, comprising a first synthetic RNA and a second synthetic RNA, wherein the first synthetic RNA encodes a gene-editing protein comprising a nuclease domain (1) capable of binding to the DNA and forming a dimer with another nuclease domain, and (2) capable of creating a single-stranded break in a DNA site of the cell, and wherein a second synthetic RNA encoding a gene-editing protein comprising a nuclease domain (1) capable of binding to the DNA and forming a dimer with another nuclease domain, and (2) incapable of creating a single-stranded break in a DNA site of the cell.

In an aspect, the present disclosure provides a composition comprising a pair of synthetic RNAs disclosed herein.

Notably, any of the gene-editing proteins disclosed herein may be adapted to provide single-stranded breaks, e.g., by including one mutated nuclease domain.

Efficient Transgene Knock-In in Human iPS Cells Combined with Small Molecule-Mediated “On-Switch” Yields Clonal Populations of Engineered Tissue-Specific Cells

Mesenchymal stem cells (MSCs) are a promising cell-therapy platform with the potential to treat a diverse array of diseases due to their immunomodulator properties—properties which can be enhanced through gene editing. Gene editing autologous or donor-derived MSCs is challenging due to the non-clonal nature of these cell sources, and associated risks of off-target effects. In contrast, induced pluripotent (iPS) cells or IPSCs, which are clonal and highly expandible, provide an ideal source of cells for gene editing and subsequent differentiation into tissue-specific cells for cell therapy applications. The present disclosure provides methods of producing clonal populations of engineered MSCs from iPS cells. Notably, the present disclosure provides control over expression of genes that are normally turned off during specific periods of differentiation. In one example, here, control relies on promoters which have temporal/stage specific regulation. By harnessing these promoters along with small molecule compounds, e.g., which demethylate DNA, a promoter can be reactivated, and genes can be turned on at specific times during a cell's differentiation pathway.

An aspect of the present disclosure is a method for activating gene expression. The method comprising obtaining a cell comprising a transgene which expresses protein of interest under the control of a promoter which is active during a defined period of a cell's differentiation and, when the cell's differentiation state is before or after the defined period during which the promoter is active, contacting the cell with a compound that demethylates DNA.

In some embodiments, the promoter has reduced activity as the cell differentiates, and the method comprises contacting the cell with the compound that demethylates DNA during differentiation to reactivate the promoter. In some cases, the promoter is inactive once the cell has differentiated, and the method comprises contacting the cell with the compound that demethylates DNA after the cell has differentiated to reactivate the promoter. In various cases, the promoter is active only as a cell differentiates, and the method comprises contacting the cell with the compound that demethylates DNA before the cell begins to differentiate to activate the promoter. In some of the foregoing embodiments, the compound that demethylates DNA is a selective histone deacetylase (HDAC) inhibitor, e.g., Trichostatin A, trapoxin, suberanilohydroxamic acid, valproate, romidepsin, or any combination thereof. In some embodiments, the compound that demethylates DNA is Trichostatin A or a functional derivative thereof. A functional derivative of a reference organic compound disclosed herein refers to an organic compound that is structurally similar to the reference organic compound with differences in one or more functional groups, and retains at least partially the desirable biological functions of the reference organic compound. A functional derivative of Trichostatin A is structurally similar to Trichostatin A and exerts inhibitory effects on HDAC signaling. In some cases, the compound that demethylates DNA is Trichostatin A.

In various embodiments, activating or reactivating the promoter induces expression of the protein of interest at an atypical period in the cell's differentiation. In some cases, expression of the protein of interest at the atypical period in the cell's differentiation changes the fate of the cell and/or changes the activity of the cell. In various cases, changing the fate of the cell comprises differentiating the cell into a cell type or cell subtype for which it was not previously destined.

In embodiments, the cell is an induced pluripotent stem cell (iPSC) which has not begun differentiating into an induced mesenchymal stem cell (iMSC). In embodiments, the cell is an induced pluripotent stem cell (iPSC) which is differentiating into an induced mesenchymal stem cell (iMSC).

In some embodiments, the promoter is a Jet promoter.

### Chemically Modified Single-Stranded DNA Donors

Genome-editing technology provides a means of modifying genes in living cells and is being explored for the development of therapies to treat cancer and a variety of genetic disorders. Gene-editing proteins can be used to create single- and double-strand breaks at specific genomic sites for knocking out a gene or, when combined with an exogenous DNA donor, knock-in of a defined sequence. In aspects of the present disclosure, provided herein is an enzymatic approach for producing long (>3 kb) and concentrated (>1 μg/μL) ssDNA suitable for generation of knock-in lines of human induced pluripotent stem (hiPS) cells.

An aspect of the present disclosure is a method for producing single-stranded DNA. The method comprising (a) obtaining a double-stranded DNA, which comprises a strand comprising at least one phosphorothioate-containing nucleotide and a strand lacking a phosphorothioate-containing nucleotide, and (b) contacting the double-stranded DNA with one or more endonucleases which digest the strand lacking the phosphorothioate-containing nucleotide, thereby obtaining a single stranded DNA.

In embodiments, the double-stranded DNA is synthesized by PCR using a standard 5′ primer lacking a phosphorothioate-containing nucleotide and a 5′ primer comprising one or more phosphorothioate-containing nucleotides, or wherein the method further comprises synthesizing the double-stranded DNA by PCR using a standard 5′ primer lacking the phosphorothioate-containing nucleotide and a 5′ primer comprising one or more phosphorothioate-containing nucleotides. In some of these embodiments, the one or more endonucleases comprise lambda exonuclease or T7 exonuclease. In some cases, the DNA is contacted with lambda exonuclease before contacting with T7 exonuclease.

In some cases, the strand comprising at least one phosphorothioate-containing nucleotide resists digestion by the one or more endonucleases. In some cases, the strand comprising at least one phosphorothioate-containing nucleotide, when used as a repair template during gene-editing, comprises the protein-coding strand of a gene.

In embodiments, the method reduces the amount of double-stranded contamination in a product comprising the single-stranded DNA by at least 80%, at least 85%, at least 90%, at least 91%, at least 92%, at least 93%, at least 94%, at least 95%, at least 96%, at least 97%, at least 98%, or at least 99%.

In embodiments, the single-stranded DNA is produced from a double stranded DNA comprising at least 30%, at least 40%, at least 50% at least 55%, at least 60%, at least 65%, at least 70%, at least 75%, at least 80% or more GC base pairs.

In embodiments, the single stranded DNA is used as a repair template in a herein-disclosed method in which a gene-editing protein creates a single stranded break in a target DNA site.

In some embodiments, the single stranded DNA is used as a repair template in the method in which a gene-editing protein creates a single stranded break in a target DNA site. The method of creating the single-stranded break may comprise (a) contacting the DNA with a first gene-editing protein comprising a nuclease domain (1) capable of binding to the DNA and forming a dimer with another nuclease domain and (2) capable of creating a single-stranded break in a DNA site; and (b) contacting the DNA with a second gene-editing protein comprising a nuclease domain (1) capable of binding to the DNA and forming a dimer with another nuclease domain and (2) incapable of creating a single-stranded break in a DNA site. In some case, nuclease domain incapable of creating a single-stranded break in a DNA site comprises a mutation in its catalytic domain. In some cases, the method further comprises contacting the DNA with a single-stranded repair template, e.g., which integrates into the DNA at the single-stranded break.

In various embodiments, the single stranded DNA provided herein is used as a repair template in the method in which a gene-editing protein creates a single stranded break in a target DNA site of a cell. The method of creating the single-stranded break may comprise creating a single-stranded break in a DNA site of a cell. The method may comprise contacting the cell with a pair of synthetic RNAs, comprising a first synthetic RNA and a second synthetic RNA, wherein the first synthetic RNA encodes a gene-editing protein comprising a nuclease domain (1) capable of binding to the DNA and forming a dimer with another nuclease domain, and (2) capable of creating a single-stranded break in a DNA site of the cell, and wherein a second synthetic RNA encoding a gene-editing protein comprising a nuclease domain (1) capable of binding to the DNA and forming a dimer with another nuclease domain, and (2) incapable of creating a single-stranded break in a DNA site of the cell. In some cases, the nuclease domain is incapable of creating a single-stranded break in a DNA site comprises a mutation in its catalytic domain. In some cases, the method further comprises contacting the cell with a single-stranded repair template, e.g., which integrates into the DNA of the cell at the single-stranded break.

Another aspect of the present disclosure is a single stranded DNA comprising at least one phosphorothioate-containing nucleotide and obtained by a method disclosed herein. In some embodiments, the at least one phosphorthioate-containing nucleotide is about 1, about 2, about 3, about 4, about 5, about 10, about 20, about 30, about 40, about 50, about 60, about 70, about 80, about 90, about 100, about 150, about 200, about 250, about 300, about 350, about 400, about 450, about 500 or more phosphorthioate-containing nucleotides.

Directed Differentiation of Gene Edited iPSCs

Indoleamine 2,3-dioxygenase 1 (IDO1) is an inducible, heme-containing enzyme that is critically involved in tryptophan catabolism and known to be a prominent immune regulator. Cell therapies with increased IDO1 expression are of high interest for a variety of indications, including autoimmune disorders, inflammatory diseases, transplant recovery, and wound healing. iPSC-derived mesenchymal stem cells (iMSCs) engineered to overexpress IDO1 may be ideal for suppressing dysregulated immune cells while simultaneously promoting expansion of regulatory T lymphocytes and the M1 (pro-inflammatory) to M2 (anti-inflammatory) polarization of macrophages.

In some cases, unfortunately, cells engineered to overexpress IDO1 have decreased proliferation rates and resist differentiating into MSCs. Therefore, the ability to produce IDO1-expressing iMSCs for suppressing dysregulated immune cells while simultaneously promoting expansion of regulatory T lymphocytes may be challenging. In one aspect, the present disclosure provides methods in which the decreased proliferation rate and prevention of differentiation caused by IDO1 expression can be minimized by providing a chemical “off switch”. Importantly, this “off switch” can allow differentiation and expansion of iMSCs. Then, once a therapeutic amount of the iMSCs is produced, the iMSCs can be administered to a subject in need thereof and IDO1 can act as an immune regulator.

Other methods for inhibiting IDO1 activity may be used. As an example, an anti-IDO1 antibody may be added to a culturing medium such that the antibody binds to IDO1 and blocks its activity.

An aspect of the present disclosure is a method for manufacturing iPSC-derived differentiated cells that are engineered to overexpress IDO1. The method comprising obtaining an iPSC comprising a genetic modification which results in overexpression of IDO1 in the iPSC, culturing the iPSC under conditions that promote differentiation of the iPSC, and contacting the iPSC with one or more IDO1 inhibitors.

In embodiments, the iPSC comprising the genetic modification which results in overexpression of IDO1 in the iPSC has delayed or non-existent differentiation absent contact with the one or more IDO1 inhibitors.

In some embodiments, the iPSC comprising the genetic modification which results in overexpression of IDO1 in the iPSC has increased population doubling time absent contact with the one or more IDO1 inhibitors.

In various embodiments, the contacting the iPSC with the one or more IDO1 inhibitors permits differentiation of the iPSC and/or reduces population doubling time of the iPSC. In various embodiments, the contacting the iPSC with the one or more IDO1 inhibitors permits differentiation of the cell. In various embodiments, the contacting the iPSC with the one or more IDO1 inhibitors reduces population doubling time of the iPSC. In various embodiments, the contacting the iPSC with the one or more IDO1 inhibitors permits differentiation of the cell and reduces population doubling time of the iPSC. In some cases, the method comprises differentiating the iPSC into a cell of the lymphoid lineage, a cell of the myeloid lineage, a hematopoietic stem cell, or a mesenchymal stromal/stem cell (MSC).

In some embodiments, the one or more IDO1 inhibitors comprise Epacadostat. In embodiments, the one or more IDO1 inhibitors comprise IDO1-IN-5. In some embodiments, the one or more IDO1 inhibitors comprise Epacadostat and IDO1-IN-5.

Another aspect of the present disclosure is a method for suppressing dysregulated immune cells while simultaneously promoting expansion of regulatory T lymphocytes and the M1 (pro-inflammatory) to M2 (anti-inflammatory) polarization of macrophages in a subject in need thereof. In some cases, the method comprises administering to the subject a therapeutically effective amount of an engineered cell wherein the engineered cell is a cell of the lymphoid lineage, a cell of the myeloid lineage, a hematopoietic stem cell, or a mesenchymal stromal/stem cell (MSC), and the engineered cell comprises a genetic modification that results in overexpression of IDO1 in the engineered cell. In embodiments engineered cell is derived from an iPSC.

Yet another aspect of the present disclosure is a pharmaceutical composition comprising a therapeutically effective amount of an engineered cell, wherein the engineered cell is a cell of the lymphoid lineage, a cell of the myeloid lineage, a hematopoietic stem cell, or a mesenchymal stromal/stem cell (MSC), and the engineered cell comprises a genetic modification that results in overexpression of IDO1 in the engineered cell.

Solid Supports Comprising iPSC-Derived Cells

An aspect of the present disclosure is a solid support comprising a therapeutically effective amount mesenchymal stromal/stem cells (MSCs), wherein the MSCs comprise a genetic modification that results in overexpression of IDO1 in the MSCs, and are derived from an iPSC.

Another aspect of the present disclosure is a method for treating a damaged organ. The method comprising contacting the damaged portion of the organ with a solid support comprising a therapeutically effective amount mesenchymal stromal/stem cells (MSCs), wherein the MSCs comprise a genetic modification that results in overexpression of IDO1 in the MSCs, and are derived from an iPSC.

Another aspect of the present disclosure is a solid support comprising a therapeutically effective amount of the mesenchymal stromal/stem cells (MSCs) manufactured by the methods disclosed herein. Another aspect of the present disclosure is a method for treating a damaged organ, the method comprising contacting a damaged portion of the damaged organ with a solid support disclosed herein.

In these aspects, the solid support, e.g., a patch or a collagen-containing sheet, holds the MSCs in proximity to the damaged organ allowing the MSCs to provide a therapeutic benefit. In some embodiments, the solid support comprises a polymer. In some embodiments, the solid support further comprises a hydrogel.

In some cases, the organ is a heart.

Methods and devices relevant to this are as described elsewhere, e.g., US20110189140A1; US20160199450A1; US20200085877A1; US20180353652A1; WO2020189948; US20220111123A1; US20220241334A1; US20220347346A1; US20220347346A1; US20230226259A1; US20230405190A1; WO2022159878; WO2022261534; WO2022267093; WO2022271255; and US20230071220A1; the contents of each of which is incorporated by reference in their entirety.

### Methods for Manufacturing Cytotoxic Lymphocytes

An aspect of the present disclosure is a method for manufacturing a population of cells that is enriched for cytotoxic lymphocytes. The method comprises steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes. In this aspect, steps (5) and (6) occur in an adherent culturing vessel. When CD34+ cells are selected, the embryoid bodies may be first chemically and/or mechanically dissociated.

In embodiments, the stem cell is an induced pluripotent stem (iPSC).

In some embodiments, the stem cell has a wild-type genome or has a genetically engineered disruption in a beta-2-microglobulin (B2M) gene. In some cases, the stem cell has a biallelic disruption in a B2M gene.

In some cases, mRNA-reprogrammed iPSC lines with a biallelic knockouts of the beta-2 microglobulin (B2M) gene, a key component of MHC class I molecules, are obtained using an mRNA-encoded chromatin context-sensitive gene-editing endonuclease. The B2M-knockout iPSCs may be differentiated using a novel, fully suspension process that replaces specialized micropatterned culture vessels with a spheroid culture step. Additional details regarding B2M knockout iPSCs useful in the present disclosure are described in PCT/US2022/019020, the contents of which are incorporated herein by reference in its entirety.

In various embodiments, the bioreactor is suited for culturing shear-sensitive cells and/or does not require use of anti-foaming agents or shear protectants, e.g., a vertical wheel bioreactor such as a PBS Biotech vertical-wheel bioreactor.

In embodiments, the medium in step (2) is serum-free and feeder-free culture medium, e.g., an mTeSR™ medium.

In some embodiments, the medium in step (6) is a serum-free and feeder-free culture medium, e.g., a StemDiff™ NK medium.

In various embodiments, the adherent culturing vessel is a multi-well plate or a cell culturing flask.

In embodiments, the method provides from about 2-fold, about 4-fold, about 6-fold, about 8-fold, about 10-fold, about 20-fold, about 40-fold, about 60-fold, about 80-fold, about 100-fold, about 200-fold, about 300-fold, about 400-fold, to about 500-fold more cytotoxic lymphocytes than obtained by a method in which each of the culturing steps comprise adherent culturing vessels; obtained by a method in which step (2) comprises a spheroid suspension culture and steps (3), (5), and (6) occur in adherent culturing vessels; and/or obtained by a method in which steps (5) and (6) occur in bioreactor.

In some embodiments, the cytotoxic lymphocytes are enriched for CD56+ cells, for CD16+ cells, NKG2D+ cells, CD226+ Cells, NKp46+ cells, NKp44+ cells, CD244+ cells, and/or CD94+ cells.

In various embodiments, the method provides from about 2-fold, about 4-fold, about 6-fold, about 8-fold, about 10-fold, about 20-fold, about 40-fold, about 60-fold, about 80-fold, about 100-fold, about 200-fold, about 300-fold, about 400-fold, to about 500-fold more CD16+ cells than obtained by a method in which step (2) comprises a spheroid suspension culture and steps (3), (5), and (6) occur in adherent culturing vessels and/or obtained by a method in which steps (5) and (6) occur in bioreactor.

In embodiments, the method provides from about 2-fold, about 4-fold, about 6-fold, about 8-fold, about 10-fold, about 20-fold, about 40-fold, about 60-fold, about 80-fold, about 100-fold, about 200-fold, about 300-fold, about 400-fold, to about 500-fold more NDG2D+ cells than obtained by a method in which step (2) comprises a spheroid suspension culture and steps (3), (5), and (6) occur in adherent culturing vessels and/or obtained by a method in which steps (5) and (6) occur in bioreactor.

In some embodiments, the method provides from about 2-fold, about 4-fold, about 6-fold, about 8-fold, about 10-fold, about 20-fold, about 40-fold, about 60-fold, about 80-fold, about 100-fold, about 200-fold, about 300-fold, about 400-fold, to about 500-fold more NKp44+ cells than obtained by a method in which step (2) comprises a spheroid suspension culture and steps (3), (5), and (6) occur in adherent culturing vessels and/or obtained by a method in which steps (5) and (6) occur in bioreactor.

In various embodiments, the method provides from about 2-fold, about 4-fold, about 6-fold, about 8-fold, about 10-fold, about 20-fold, about 40-fold, about 60-fold, about 80-fold, about 100-fold, about 200-fold, about 300-fold, about 400-fold, to about 500-fold more CD94+ cells than obtained by a method in which step (2) comprises a spheroid suspension culture and steps (3), (5), and (6) occur in adherent culturing vessels and/or obtained by a method in which steps (5) and (6) occur in bioreactor.

In embodiments, the method provides from about 2-fold, about 4-fold, about 6-fold, about 8-fold, about 10-fold, about 20-fold, about 40-fold, about 60-fold, about 80-fold, about 100-fold, about 200-fold, about 300-fold, about 400-fold, to about 500-fold more NKp46 cells than obtained by a method in which step (2) comprises a spheroid suspension culture and steps (3), (5), and (6) occur in adherent culturing vessels.

In some embodiments, the cytotoxic lymphocyte targets and kills cancer cells, e.g., a K562 cancer cell. In various embodiments, the cytotoxic lymphocyte targets and kills cancer cells without requiring IL-15 and/or without requiring IL-2 activation. In embodiments, the cytotoxic lymphocyte targets and kills at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or more cancer cells in a population within about 4 hours. In some embodiments, the cytotoxic lymphocyte targets and kills at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or more of cancer cells in a population within about 24 hours.

In various embodiments, the cytotoxic lymphocyte has reduced cytotoxicity to an NK-resistant cancer cell, e.g., a NAMALWA cell.

In embodiments, the cytotoxic lymphocyte is a Natural Killer (NK) cell. In some cases, the NK cell is a mature NK cell.

In some embodiments, the cytotoxic lymphocyte is a Natural killer T (NKT) cell.

In various embodiments, the cytotoxic lymphocyte is a delta-gamma T cell.

In various embodiments, the cytotoxic lymphocyte is an alpha beta T cell.

In various embodiments, the cytotoxic lymphocyte is a CD4 T cell.

In various embodiments, the cytotoxic lymphocyte is a CD8 T cell.

In embodiments, the iPSC was reprogrammed from a somatic cell by contacting the somatic cell with one or more ribonucleic acids (RNAs), wherein each RNA encodes one or more reprogramming factors.

### Cytotoxic Lymphocytes

In embodiments, the present cytotoxic lymphocyte is of the lymphoid cell lineage or the myeloid cell lineage.

In some cases, the lymphoid cell is a T cell, e.g., a cytotoxic T cell or gamma-delta T cell.

In some cases, the lymphoid cell is an NK cell, e.g., an NK-T cell. The NK cell may be a human cell.

In some cases, the myeloid cell is a macrophage, e.g., an M1 macrophage or an M2 macrophage.

In various embodiment, the cytotoxic lymphocyte is reprogrammed from a stem cell, e.g., an iPSC, and differentiated into the cytotoxic lymphocyte.

In embodiments, the cytotoxic lymphocyte has a disruption in its beta-2-microglobulin (B2M) gene.

In embodiments, the cytotoxic lymphocyte has a disruption in its beta-2-microglobulin (B2M) gene and expresses a fusion protein comprising a B2M polypeptide and an HLA polypeptide (e.g., an HLA-A, HLA-B, HLA-C, HLA-E, HLA-F and HLA-G polypeptide).

In embodiments, the cytotoxic lymphocyte is gene edited to express a high affinity variant of CD16a.

In embodiments, the myeloid lineage cell is a cell derived from, or derivable from, a common myeloid progenitor cell. In embodiments, the myeloid cell is a megakaryocyte, erythrocyte, mast cell, or myeloblast. In embodiments, the myeloid cell is a cell derived from, or derivable from, a myeloblast. In embodiments, the myeloid cell is a basophil, neutrophil, eosinophil, or monocyte. In embodiments, the myeloid cell is a cell derived from, or derivable from a monocyte. In embodiments, the myeloid cell is a macrophage. In embodiments, the myeloid cell is a dendritic cell.

In embodiments, the cytotoxic lymphocyte is an NK cell. In embodiments, the NK cell is a human cell. In embodiments, the NK cell is derived from somatic cell of a subject. In embodiments, the NK cell is derived from allogeneic or autologous cells. In embodiments, the NK cell is derived from an induced pluripotent stem (iPS) cell. In embodiments, the iPS is derived from reprogramming a somatic cell to an iPS cell, the reprogramming comprising contacting the iPS cell with a ribonucleic acid (RNA) encoding one or more reprogramming factors, optionally selected from Oct4, Sox2, cMyc, and Klf4. In embodiments, the iPS cell is derived from allogeneic or autologous cells. In embodiments, the NK cell expresses one or more of CD56 and CD16.

In embodiments, the NK cell expresses CD16a, which optionally binds an antibody/antigen complex on a tumor cell and/or wherein the CD16a is optionally a high affinity variant, optionally homozygous or heterozygous for F158V.

In embodiments, the NK cell does not express CD3.

In embodiments, the NK cell is CD56^{bright} CD16^{dim/-}. In embodiments, the NK cell is CD56^{dim} CD16+. In embodiments, the NK cell is a NK^{tolerant} cell, optionally comprising CD56^{bright} NK cells or CD27− CD11b− NK cells. In embodiments, the NK cell is a NK^{cytotoxic}, optionally comprising CD56^{dim} NK cells or CD11b+ CD27− NK cells. In embodiments, the NK cell is a NK^{regulatory} cell, optionally comprising CD56^{bright} NK cells or CD27+ NK cells. In embodiments, the NK cell is a natural killer T (NKT) cell.

In embodiments, the NK cell secretes one or more cytokines selected from interferon-gamma (IFN-g), tumor necrosis factor-alpha (TNF-a), tumor necrosis factor-beta (TNF-b), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b).

In embodiments, the present cytotoxic lymphocyte has reduced or eliminated cytotoxic lymphocyte fratricide, e.g., NK-cell fratricide. For instance, in embodiments, the present engineered NK cells surprisingly do not engage in NK cytotoxicity and therefore are able to survive despite disruptions, e.g., in beta-2-microglobulin (B2M).

In embodiments, the present cytotoxic lymphocyte is capable of self-activating. In embodiments, the present cytotoxic lymphocyte is capable of activating without the need for extracellular signals (e.g., cytokines), including signals that may be provided exogenously. In embodiments, the present cytotoxic lymphocyte does not require ex vivo stimulation for activity. In embodiments, the present cytotoxic lymphocyte is capable of self-activating in the absence of an interleukin, optionally selected from IL-2 and IL-15.

In embodiments, the present cytotoxic lymphocyte is capable of inducing tumor cell cytotoxicity. In embodiments, the present cytotoxic lymphocyte is capable of inducing tumor cell cytotoxicity in the absence of an interleukin, optionally selected from IL-2 and IL-15. Assays for assessing tumor cell cytotoxicity include in vivo anti-cancer response evaluation, as well as microscopic evaluation, e.g., a calcein acetoxymethyl (AM) staining-based microscopic method (See EXAMPLES and Chava et al. J Vis Exp. 2020 Feb. 22; (156): 10.3791/60714, the entire contents of which are incorporated by reference). Further, a colorimetric lactic dehydrogenase (LDH) measurement-based NK cell-mediated cytotoxicity assay may be employed (see Chava et al. J Vis Exp. 2020 Feb. 22; (156): 10.3791/60714, the entire contents of which are incorporated by reference).

Scalable, Mixed Population iPS-Cell Derived Cytotoxic Lymphocytes and Myeloid Cells

Induced pluripotent stem cell (iPSC) therapies have the potential to treat a wide variety of devastating diseases. iPSC-derived lymphocytes (e.g., T cells and NK cells) engineered to express targeting molecules such as chimeric antigen receptors (CARs) have shown clinical promise to treat hematological malignancies. More recently, iPS cell-derived myeloid cells are being developed to treat both hematological malignancies and solid tumors due to the ability of these cells to infiltrate and modulate the tumor microenvironment. Despite preliminary success, several challenges still remain, including poor infiltration of cytotoxic lymphocytes into solid tumors and insufficient cytotoxicity of myeloid cells.

As is known in the art, an animal's immune system comprises a wide variety of immune cell types capable of contributing to an anti-cancer effect. And, in vivo, one type of immune cell promotes the cancer-killing ability of a second type of immune cell. Notably, NK cells are expert in killing cancer cells but rarely infiltrate solid tumors alone and require recruitment by macrophages which have already infiltrated the solid tumor and, on the other hand, macrophages are less adept at killing cancer cells but expert in infiltrating solid tumors and secreting cytokines that recruit cancer killing cells. Thus, each type of immune cell has its function which work in cooperation with the other cell types to attack and kill cancer cells. Nonetheless, many cell-based cancer therapeutics in clinical trials employ one type of immune cell rather than a plurality of immune cell types as existent in vivo. Without wishing to be bound by theory, a multi-cell-type therapy comprising both lymphocyte and myeloid cells may work synergistically, enhancing cytotoxicity and efficacy.

This disclosure, in some aspects, provides a scalable platform for generating iPSC-derived multi-cell-type therapies comprising both lymphoid and myeloid cells. These cells act synergistically to kill tumor cells in vitro. And, by closely mimicking natural cellular immunity, multi-cell-type cell therapies represent a new class of cell therapies that may play an important role in the development of new medicines for treating cancer.

An aspect of the present disclosure is a method for treating a cancer. The method comprising administering to a subject in need a therapeutically-effective amount of a first pharmaceutical composition comprising one or both of a population of isolated lymphoid lineage cells and a population of isolated myeloid lineage cells.

In embodiments, the first pharmaceutical composition comprises the population of isolated lymphoid lineage cells and wherein the subject in need is administered a therapeutically-effective amount of a second pharmaceutical composition comprising a population of isolated myeloid lineage cells.

In some embodiments, the first pharmaceutical composition comprises the population of isolated myeloid lineage cells and wherein the subject in need is administered a therapeutically-effective amount of a second pharmaceutical composition comprising a population of isolated lymphoid lineage cells. In some cases, the first pharmaceutical composition and the second pharmaceutical composition are administered simultaneously or sequentially. The first pharmaceutical composition and the second pharmaceutical composition may be administered sequentially with the first pharmaceutical composition administered before the second pharmaceutical composition or the first pharmaceutical composition and the second pharmaceutical composition may be administered sequentially with the second pharmaceutical composition administered before the first pharmaceutical composition.

In various embodiments, the first pharmaceutical composition comprises both the population of isolated lymphoid lineage cells and the population of isolated myeloid lineage cells.

In numerous embodiments, one or more of the isolated lymphoid lineage cells and/or one or more of the isolated myeloid lineage cells comprise a genomic modification that expresses a chimeric antigen receptor (CAR), e.g., a CAR which comprises an antigen binding region that binds to one or more antigens expressed by a cancer cell. In some cases, the antigen binding region binds to one or more tumor antigens. In various cases, the CAR comprises an antigen binding region that binds to ROR1.

In embodiments, one or more of the isolated lymphoid lineage cells and/or one or more of the isolated myeloid lineage cells comprise a genomic modification that expresses or over expresses a cytokine.

In some embodiments, one or more of the isolated lymphoid lineage cells and/or one or more of the isolated myeloid lineage cells comprise a genomic modification which disrupts the beta-2-microglobulin (B2M) gene, optionally, wherein the cells express a fusion protein comprising a B2M polypeptide and an HLA polypeptide (e.g., an HLA-A, HLA-B, HLA-C, HLA-E, HLA-F and HLA-G polypeptide).

In various embodiments, the method further comprises administering to the subject in need a synthetic mRNA encoding a gene-editing protein (e.g., a temperature-sensitive gene-editing protein) and a single-stranded or double-stranded repair template which encodes a chimeric antigen receptor (CAR). In some cases, the gene-editing protein creates a single-stranded break or a double-stranded break in the genomic DNA of a cell in the subject and the single-stranded or double-stranded repair template which encodes the CAR inserts into the break. In these embodiments, the cell in the subject expresses the CAR.

In numerous embodiments, the method further comprises administering to the subject in need a synthetic mRNA encoding a gene-editing protein (e.g., a temperature-sensitive gene-editing protein) and a single-stranded or double-stranded repair template which encodes a cytokine. In some cases, the gene-editing protein creates a single-stranded break or a double-stranded break in the genomic DNA of a cell in the subject and the single-stranded or double-stranded repair template which encodes the cytokine inserts into the break. In these embodiments, the cell in the subject expresses or over expresses the cytokine.

In some embodiments, when the synthetic mRNA and/or the repair template is administered to a subject, the synthetic mRNA and/or the repair is combined with a lipid system comprising a compound of Formula (IV).

In various cases, transfection of a cell with synthetic nucleic acids for gene-editing may be facilitated by use of the ToRNAdo™ Nucleic-Acid Delivery System. This system relates to new lipids that find use, inter alia, in improved delivery of biological payloads, e.g., nucleic acids, to cells. The system relates to use of a compound of Formula (IV)

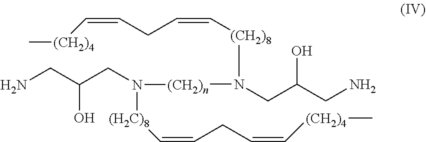

where n is 1, 2, 3, 4, 5, 6, 7, 8, 9, 10, 11, 12, 13, 14, or 15. Further description of ToRNAdo™ Nucleic-Acid Delivery System is found in one or both of US. Pat. No. 10,501,404 and US20210009505A1. The entire contents of which are incorporated by reference in their entirety.

In embodiments, the cell in the subject (e.g., which expresses the CAR and/or cytokine) is of the lymphoid lineage or is of the myeloid lineage.

In some embodiments, the isolated lymphoid lineage cell and/or the isolated myeloid lineage cell is derived from an induced pluripotent stem cell (iPSC). In some cases, the isolated lymphoid lineage cell and the isolated myeloid lineage cell is derived from the same iPSC. The iPSC comprises a genomic modification that expresses a chimeric antigen receptor (CAR) and/or the iPSC comprises a genomic modification that expresses or over expresses a cytokine. In various cases, the iPSC comprises a genomic modification which disrupts the beta-2-microglobulin (B2M) gene.

In various embodiments, the isolated lymphoid lineage cells are manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a lymphoid progenitor medium; and (6) culturing the cells of step (5) in an NK cell medium under conditions to obtain a population of cells enriched for cytotoxic lymphocytes; wherein steps (5) and (6) occur in an adherent culturing vessel.

In numerous embodiments, the isolated lymphoid lineage cells comprise cytotoxic lymphocytes. In some cases, the isolated lymphoid lineage cells comprising cytotoxic lymphocytes are enriched for CD56+ cells, for CD16+ cells, NKG2D+ cells, CD226+ Cells, NKp46+ cells, NKp44+ cells, CD244+ cells, and/or CD94+ cells. In these embodiments, the cytotoxic lymphocyte targets and kills cancer cells and, in some cases, the cytotoxic lymphocyte targets and kills cancer cells without requiring IL-15 and/or without requiring IL-2 activation. The cytotoxic lymphocyte has reduced cytotoxicity to an NK-resistant cancer cell. In various cases, the cytotoxic lymphocyte is a Natural Killer (NK) cell, e.g., a mature NK cell, or is a cytotoxic T cell. In these embodiments, the cytotoxic lymphocyte is a Natural killer T (NKT) cell. In some cases, the NK cell expresses CD16a and/or the NK cell does not express CD3, and/or the NK cell is CD56bright CD16dim/−. In many cases, the NK cell secretes one or more cytokines selected from interferon-gamma (IFNγ), tumor necrosis factor-alpha (TNFα), tumor necrosis factor-beta (TNFβ), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b). The cytotoxic lymphocyte may be a delta-gamma T cell. In numerous cases, the cytotoxic lymphocyte is further engineered to express a chimeric antigen receptor (CAR) and/or is further engineered to express or overexpress a cytokine.

In embodiments, the isolated myeloid lineage cells are manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5) culturing the CD34+ cells in a myeloid progenitor medium; and (6) culturing the cells of step (5) in a macrophage cell medium under conditions to obtain a population of cells enriched for macrophages; wherein steps (5) and (6) occur in a bioreactor.

In some embodiments, the isolated myeloid lineage cells comprise a megakaryocyte, erythrocyte, mast cell, myeloblast, dendritic cell, basophil, neutrophil, eosinophil, monocyte, or macrophage.

In various embodiments, the isolated myeloid lineage cells express one or more of CD11b, CD13, CD14, CD33, CD45, CD80, CD163, CD206, and SIRPα, e.g., in amounts that are similar to amounts expressed by PBMC-derived cells.

In numerous embodiments, the isolated myeloid lineage cells have increased expression of CD80 and/or CD206, which is indicative of an activated state.

In embodiments, the isolated myeloid lineage cell is a macrophage. In some cases, the macrophage expresses one or more of CD11b, CD68, CD80, CD86, CD163, CD206, and SIRPα in amounts that are similar to amounts expressed by PBMC-derived cells and/or secretes one or more of TNFα, IL-12p70, and IL-10 in amounts that are similar to amounts expressed by PBMC-derived cells. In various cases, the macrophage expresses one or more of CD34, CD44, CD45, CD73, and CD90. In some embodiments, the method further comprises a step of differentiating the macrophages into M1 and/or M2 macrophages, e.g., by exposure to MCSF. And, the method may further comprise a step of polarizing the M1 macrophages with interferon gamma (IFN-γ) and lipopolysaccharide (LPS) and/or treating the M2 macrophages with IL-4. In these cases, the macrophages comprise M1 macrophages and/or M2 macrophages. The M1 macrophages and/or M2 macrophages secrete one or more of TNFα, IL-12p70, and IL-10 in amounts that are similar to amounts expressed by PBMC-derived cells.

In some embodiments, the isolated myeloid lineage cells kill cancer cells and/or promote cancer cell killing by cytotoxic lymphocytes.

In various embodiments, the isolated myeloid lineage cell is further engineered to express a chimeric antigen receptor (CAR).

In numerous embodiments, the isolated myeloid lineage cell is further engineered to express or overexpress a cytokine. In cases when CD34+ cells are selected, the embryoid bodies are first chemically and/or mechanically dissociated.

In embodiments, the stem cell is an induced pluripotent stem (iPSC). In some cases, the stem cell stem has a wild-type genome or has a genetically engineered disruption in a beta-2-microglobulin (B2M) gene, e.g., a biallelic disruption in a B2M gene. In various cases, the stem cell expresses a fusion protein comprising a B2M polypeptide and an HLA polypeptide (e.g., an HLA-A, HLA-B, HLA-C, HLA-E, HLA-F and HLA-G polypeptide).

In some embodiments, the iPSC was reprogrammed from a somatic cell and the method further comprises contacting the somatic cell with one or more ribonucleic acids (RNAs), wherein each RNA encodes one or more reprogramming factors. The one or more reprogramming factors may be selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In some cases, the somatic cell is selected from fibroblasts, keratinocytes, melanocyte blood cells, bone marrow cells, adipose cells, and tissue-resident progenitor cells.

In embodiments where the stem cell is an iPSC, the iPSC is further engineered to express a chimeric antigen receptor (CAR) and/or the iPSC is further engineered to express or overexpress a cytokine.

In various embodiments, the isolated lymphoid lineage cells and the isolated myeloid lineage cells are manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor. In cases when CD34+ cells are selected, the embryoid bodies are first chemically and/or mechanically dissociated. In various cases, the stem cell is an induced pluripotent stem (iPSC).

In embodiments, the method of manufacturing provides at least 1×10⁶ myeloid lineage cells/ml and at least 3×10⁵ lymphoid lineage cells/ml.

In some embodiments, the method of manufacturing provides both CD14+ (>95% positive) macrophages and CD56^{bright}/CD16^{dim} NK cells.

In various embodiments, the method of manufacturing is amenable to scaling to clinically relevant doses.

In embodiments, the population of isolated lymphoid lineage cells and the population of isolated myeloid lineage cells act synergistically to kill cancer cells.

In numerous embodiments, the administering is intravenous, intraarterial, intratumoral, or injected in the vicinity of a tumor.

In embodiments, the cancer is a blood cancer.

In some embodiments, the cancer is a solid tumor.

In various embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

Another aspect of the present disclosure is a plurality of compositions for use in any herein-disclosed method for treating a cancer.

Yet another aspect of the present disclosure is a method for killing a cancer cell or for inhibiting the proliferation of a cancer cell. The method comprising contacting the cancer cell with a population of isolated lymphoid lineage cells and a population of isolated myeloid lineage cells.

In numerous embodiments, the cancer cell is contacted with the population of isolated lymphoid lineage cells and the population of isolated myeloid lineage cells simultaneously.

In embodiments, the cancer cell is contacted with the population of isolated lymphoid lineage cells before being contacted with the population of isolated myeloid lineage cells or the cancer cell is contacted with the population of isolated lymphoid lineage cells after being contacted with the population of isolated myeloid lineage cells.

In some embodiments, wherein one or more of the isolated lymphoid lineage cells and/or one or more of the isolated myeloid lineage cells comprise a genomic modification that expresses a chimeric antigen receptor (CAR). In some cases, the CAR comprises an antigen binding region that binds to one or more antigens expressed by a cancer cell. In various cases, the antigen binding region binds to one or more tumor antigens. In these embodiments, the CAR may comprise an antigen binding region that binds to ROR1.

In various embodiments, one or more of the isolated lymphoid lineage cells and/or one or more of the isolated myeloid lineage cells comprise a genomic modification that expresses or over expresses a cytokine.

In numerous embodiments, one or more of the isolated lymphoid lineage cells and/or one or more of the isolated myeloid lineage cells comprise a genomic modification which disrupts the beta-2-microglobulin (B2M) gene, optionally, wherein the cells express a fusion protein comprising a B2M polypeptide and an HLA polypeptide (e.g., an HLA-A, HLA-B, HLA-C, HLA-E, HLA-F and HLA-G polypeptide).

In embodiments, the isolated lymphoid lineage cells comprise cytotoxic lymphocytes. In some cases, the isolated lymphoid lineage cells comprising cytotoxic lymphocytes are enriched for CD56+ cells, for CD16+ cells, NKG2D+ cells, CD226+ Cells, NKp46+ cells, NKp44+ cells, CD244+ cells, and/or CD94+ cells. In various cases, the cytotoxic lymphocyte targets and kills cancer cells, e.g., the cytotoxic lymphocyte targets and kills cancer cells without requiring IL-15 and/or without requiring IL-2 activation. The cytotoxic lymphocyte has reduced cytotoxicity to an NK-resistant cancer cell. In some cases, the cytotoxic lymphocyte is a Natural Killer (NK) cell, e.g., a mature NK cell, or is a cytotoxic T cell. The cytotoxic lymphocyte may be a Natural killer T (NKT) cell. In these embodiments, the NK cell expresses CD16a and/or the NK cell does not express CD3 and/or the NK cell is CD56bright CD16dim/−. In some cases, the NK cell secretes one or more cytokines selected from interferon-gamma (IFNγ), tumor necrosis factor-alpha (TNFα), tumor necrosis factor-beta (TNFβ), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b). In other case, the cytotoxic lymphocyte is a delta-gamma T cell. In these embodiments, the cytotoxic lymphocyte is further engineered to express a chimeric antigen receptor (CAR) and/or is further engineered to express or overexpress a cytokine.

In some embodiments, the isolated myeloid lineage cells comprise a megakaryocyte, erythrocyte, mast cell, myeloblast, dendritic cell, basophil, neutrophil, eosinophil, monocyte, or macrophage.

In various embodiments, the isolated myeloid lineage cells express one or more of CD11b, CD13, CD14, CD33, CD45, CD80, CD163, CD206, and SIRPα, e.g., in amounts that are similar to amounts expressed by PBMC-derived cells.

In numerous embodiments, the isolated myeloid lineage cells have increased expression of CD80 and/or CD206, which is indicative of an activated state.

In embodiments, the isolated myeloid lineage cell is a macrophage. In some cases, the macrophage expresses one or more of CD11b, CD68, CD80, CD86, CD163, CD206, and SIRPα in amounts that are similar to amounts expressed by PBMC-derived cells and/or secretes one or more of TNFα, IL-12p70, and IL-10 in amounts that are similar to amounts expressed by PBMC-derived cells. The macrophage may express one or more of CD34, CD44, CD45, CD73, and CD90. In various cases, the macrophages comprise M1 macrophages and/or M2 macrophages. The M1 macrophages and/or M2 macrophages may secrete one or more of TNFα, IL-12p70, and IL-10 in amounts that are similar to amounts expressed by PBMC-derived cells.

In some embodiments, the isolated myeloid lineage cells kill cancer cells and/or promote cancer cell killing by cytotoxic lymphocytes.

In various embodiments, the isolated myeloid lineage cell is further engineered to express a chimeric antigen receptor (CAR).

In numerous embodiments, the isolated myeloid lineage cell is further engineered to express or overexpress a cytokine.

In embodiments, the isolated lymphoid lineage cells and the isolated myeloid lineage cells are manufactured by a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor. In cases when CD34+ cells are selected, the embryoid bodies are first chemically and/or mechanically dissociated. In these embodiments, the stem cell may be an induced pluripotent stem (iPSC). In some cases, the isolated lymphoid lineage cell and the isolated myeloid lineage cell are derived from the same iPSC. In various cases, the iPSC comprises a genomic modification that expresses a chimeric antigen receptor (CAR) and/or comprises a genomic modification that expresses or over expresses a cytokine. The iPSC may comprise a genomic modification which disrupts the beta-2-microglobulin (B2M) gene, e.g., a biallelic disruption in a B2M gene; in these cases, the iPSC expresses a fusion protein comprising a B2M polypeptide and an HLA polypeptide (e.g., an HLA-A, HLA-B, HLA-C, HLA-E, HLA-F and HLA-G polypeptide).

In embodiments, the iPSC was reprogrammed from a somatic cell, and the method further comprises contacting the somatic cell with one or more ribonucleic acids (RNAs), wherein each RNA encodes one or more reprogramming factors. The one or more reprogramming factors may be selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In some cases, the somatic cell is selected from fibroblasts, keratinocytes, melanocyte blood cells, bone marrow cells, adipose cells, and tissue-resident progenitor cells.

In some embodiments, contacting the cancer cell with a population of isolated lymphoid lineage cells and a population of isolated myeloid lineage cells occurs in vitro.

In various embodiments, contacting the cancer cell with a population of isolated lymphoid lineage cells and a population of isolated myeloid lineage cells occurs in vivo.

Yet a further aspect of the present disclosure is a plurality of compositions for use in any herein-disclosed method for killing a cancer cell or for inhibiting the proliferation of a cancer cell.

In an aspect, the present disclosure provides a method for manufacturing a plurality of population of cells comprising a population of isolated lymphoid lineage cells and a population of isolated myeloid lineage cells for treating a cancer, for killing a cancer cell, and/or for inhibiting the proliferation of a cancer cell. The method a method comprising steps of (1) obtaining a stem cell; (2) culturing the stem cell in a bioreactor comprising a media that promotes formation of spheroids; (3) culturing the spheroids in a bioreactor in a media that promotes formation of embryoid bodies; (4) optionally, selecting CD34+ cells from the embryoid bodies; (5a) culturing a first subset of the CD34+ cells in a lymphoid progenitor medium and (5b) culturing a second subset of the CD34+ cells in a myeloid progenitor medium; (6a) culturing the cells of step (5a) in an NK cell medium under conditions to obtain a population of cells enriched for cytotoxic lymphocytes and (6b) culturing the cells of step (5b) in a macrophage cell medium under conditions to obtain a population of cells enriched for macrophages; wherein steps (5a) and (6a) occur in an adherent culturing vessel, and steps (5b) and (6b) occur in a bioreactor.

In numerous embodiments, when CD34+ cells are selected, the embryoid bodies are first chemically and/or mechanically dissociated.

In embodiments, the stem cell is an induced pluripotent stem (iPSC). In some cases, the isolated lymphoid lineage cell and the isolated myeloid lineage cell are derived from the same iPSC. The iPSC may comprise a genomic modification that expresses a chimeric antigen receptor (CAR) and/or a genomic modification that expresses or over expresses a cytokine. In various cases, the iPSC comprises a genomic modification which disrupts the beta-2-microglobulin (B2M) gene, e.g., a biallelic disruption in a B2M gene. In these cases, the iPSC expresses a fusion protein comprising a B2M polypeptide and an HLA polypeptide (e.g., an HLA-A, HLA-B, HLA-C, HLA-E, HLA-F and HLA-G polypeptide).

In some embodiments, the iPSC was reprogrammed from a somatic cell, and the method further comprises contacting the somatic cell with one or more ribonucleic acids (RNAs), wherein each RNA encodes one or more reprogramming factors. The one or more reprogramming factors may be selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In some case, the somatic cell is selected from fibroblasts, keratinocytes, melanocyte blood cells, bone marrow cells, adipose cells, and tissue-resident progenitor cells.

In various embodiments, one or more of the isolated lymphoid lineage cells and/or one or more of the isolated myeloid lineage cells comprise a genomic modification that expresses a chimeric antigen receptor (CAR), e.g., which comprises an antigen binding region that binds to one or more antigens expressed by a cancer cell. In some case, the antigen binding region binds to one or more tumor antigens. In these embodiments, the CAR may comprise an antigen binding region that binds to ROR1.

In numerous embodiments, one or more of the isolated lymphoid lineage cells and/or one or more of the isolated myeloid lineage cells comprise a genomic modification that expresses or over expresses a cytokine.

In embodiments, one or more of the isolated lymphoid lineage cells and/or one or more of the isolated myeloid lineage cells comprise a genomic modification which disrupts the beta-2-microglobulin (B2M) gene. In some cases, the cells express a fusion protein comprising a B2M polypeptide and an HLA polypeptide (e.g., an HLA-A, HLA-B, HLA-C, HLA-E, HLA-F and HLA-G polypeptide).

In some embodiments, the isolated lymphoid lineage cells comprise cytotoxic lymphocytes. In some cases, the isolated lymphoid lineage cells comprising cytotoxic lymphocytes are enriched for CD56+ cells, for CD16+ cells, NKG2D+ cells, CD226+ Cells, NKp46+ cells, NKp44+ cells, CD244+ cells, and/or CD94+ cells. The cytotoxic lymphocyte targets and kills cancer cells. In various cases, the cytotoxic lymphocyte targets and kills cancer cells without requiring IL-15 and/or without requiring IL-2 activation. The cytotoxic lymphocyte may have reduced cytotoxicity to an NK-resistant cancer cell. The cytotoxic lymphocyte may be a Natural Killer (NK) cell, e.g., a mature NK cell, or is a cytotoxic T cell. The cytotoxic lymphocyte may be a Natural killer T (NKT) cell. The NK cell may express CD16a and/or the NK cell does not express CD3 and/or the NK cell is CD56bright CD16dim/−. In various cases, the NK cell secretes one or more cytokines selected from interferon-gamma (IFNγ), tumor necrosis factor-alpha (TNFα), tumor necrosis factor-beta (TNFβ), granulocyte macrophage-colony stimulating factor (GM-CSF), interleukin-2 (IL-2), interleukin-7 (IL-7), interleukin-10 (IL-10), interleukin-13 (IL-13), macrophage inflammatory protein-1a (MIP-1a), and macrophage inflammatory protein-1b (MIP-1b). The cytotoxic lymphocyte may be a delta-gamma T cell. In some cases, the cytotoxic lymphocyte is further engineered to express a chimeric antigen receptor (CAR) and/or is further engineered to express or overexpress a cytokine.

In various embodiments, the isolated myeloid lineage cells comprise a megakaryocyte, erythrocyte, mast cell, myeloblast, dendritic cell, basophil, neutrophil, eosinophil, monocyte, or macrophage.

In numerous embodiments, the isolated myeloid lineage cells express one or more of CD11b, CD13, CD14, CD33, CD45, CD80, CD163, CD206, and SIRPα, e.g., in amounts that are similar to amounts expressed by PBMC-derived cells.

In embodiments, the isolated myeloid lineage cells have increased expression of CD80 and/or CD206, which is indicative of an activated state.

In some embodiments, the isolated myeloid lineage cell is a macrophage. In some cases, the macrophage expresses one or more of CD11b, CD68, CD80, CD86, CD163, CD206, and SIRPα in amounts that are similar to amounts expressed by PBMC-derived cells and/or secretes one or more of TNFα, IL-12p70, and IL-10 in amounts that are similar to amounts expressed by PBMC-derived cells. In various cases, the macrophage expresses one or more of CD34, CD44, CD45, CD73, and CD90. In some embodiments, the method may further comprise a step of differentiating the macrophages into M1 and/or M2 macrophages, e.g., by exposure to MCSF. The method may also further comprise a step of polarizing the M1 macrophages with interferon gamma (IFN-γ) and lipopolysaccharide (LPS) and/or treating the M2 macrophages with IL-4. In various cases, the macrophages comprise M1 macrophages and/or M2 macrophages; the M1 macrophages and/or M2 macrophages may secrete one or more of TNFα, IL-12p70, and IL-10 in amounts that are similar to amounts expressed by PBMC-derived cells.

In various embodiments, the isolated myeloid lineage cells kill cancer cells and/or promote cancer cell killing by cytotoxic lymphocytes.

In numerous embodiments, the isolated myeloid lineage cell is further engineered to express a chimeric antigen receptor (CAR).

In embodiments, the isolated myeloid lineage cell is further engineered to express or overexpress a cytokine.

In some embodiments, the iPSC was contacted with resveratrol before reprogramming.

In various embodiments, one or more culturing steps comprise a medium which is serum-free culture medium and/or feeder-free culture medium. In some cases, the serum-free culture medium and/or feeder-free culture medium is an mTeSR™ medium and/or the serum-free culture medium and/or feeder-free culture medium is a StemDiff™ NK medium.

Innumerous embodiments, the adherent culturing vessel is a multi-well plate or a cell culturing flask.

In embodiments, the method of manufacturing provides at least 1×10⁶ myeloid lineage cells/ml and at least 3×10⁵ lymphoid lineage cells/ml.

In some embodiments, the method of manufacturing provides both CD14+ (>95% positive) macrophages and CD56^{bright}/CD16^{dim} NK cells.

In various embodiments, the method of manufacturing is amenable to scaling to clinically relevant doses.

In embodiments, the population of isolated lymphoid lineage cells and the population of isolated myeloid lineage cells act synergistically to kill cancer cells.

In embodiments, the cancer is a blood cancer.

In some embodiments, the cancer is a solid tumor.

In various embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome.

In another aspect, the present disclosure provides a plurality population of cells comprising a population of isolated lymphoid lineage cells and a population of isolated myeloid lineage cells for treating a cancer, for killing a cancer cell, and/or for inhibiting the proliferation of a cancer cell which were manufactured by any herein-disclosed method.

### Chimeric Antigen Receptor (CAR)-Bearing Cytotoxic Lymphocytes

In embodiments, the present cytotoxic lymphocytes are engineered with chimeric antigen receptors (CARs), e.g., the present cytotoxic lymphocytes are CAR-NK cells, CAR-T cells, or CAR-myeloid cells.

In embodiments, a cell is genetically modified to express a recombinant chimeric antigen receptor (CAR) comprising an intracellular signaling domain, a transmembrane domain, and an extracellular domain comprising an antigen binding region. In embodiments, the intracellular signaling domain comprises at least one immune receptor tyrosine-based activation motif (ITAM)-containing domain.

In embodiments, the intracellular signaling domain is from one of CD3-zeta, CD28, CD27, CD134 (OX40), and CD137 (4-1BB).

In embodiments, the transmembrane domain is from one of CD28 or a CD8.

In embodiments, the antigen binding region binds one antigen. In embodiments, the binding region binds two antigens.

In embodiments, the extracellular domain comprising an antigen binding region comprises: (a) a natural ligand or receptor, or fragment thereof, or (b) an immunoglobulin domain, optionally a single-chain variable fragment (scFv). In embodiments, the extracellular domain comprising an antigen binding region comprises two of (a) a natural ligand or receptor, or fragment thereof, or (b) an immunoglobulin domain, optionally a single-chain variable fragment (scFv). In embodiments, the extracellular domain comprising an antigen binding region comprises one of each of (a) a natural ligand or receptor, or fragment thereof, and (b) an immunoglobulin domain, optionally a single-chain variable fragment (scFv).

In embodiments, the antigen binding region binds a tumor antigen.

In embodiments, the antigen binding region comprises one or more of (i) CD94/NKG2a, which optionally binds HLA-E on a tumor cell; (ii) CD96, which optionally binds CD155 on a tumor cell; (iii) TIGIT, which optionally binds CD155 or CD112 on a tumor cell; (iv) DNAM-1, which optionally binds CD155 or CD112 on a tumor cell; (v) KIR, which optionally binds HLA class I on a tumor cell; (vi) NKG2D, which optionally binds NKG2D-L on a tumor cell; (vii) CD16 (e.g., CD16a or CD16b), which optionally binds an antibody/antigen complex on a tumor cell and/or wherein the CD16a is optionally a high affinity variant, optionally homozygous or heterozygous for F158V; (viii) NKp30, which optionally binds B7-H6 on a tumor cell; (ix) NKp44; and (x) NKp46.

In embodiments, the antigen binding region comprises an immunoglobulin domain, optionally an scFv directed against HLA-E, CD155, CD112 HLA class I, NKG2D-L, or B7-H6, as well as any variant thereof.

In embodiments, the antigen binding region binds an antigen, e.g., a tumor antigen, selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, as well as any variant thereof. In embodiments, an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, as well as any variant thereof can be used as a single-target CAR, dual-target CAR, mAb, or any combination of any of those.

In embodiments, the antigen binding region binds two antigen, e.g., two tumor antigens, the antigens being: (a) an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2, as well as any variant thereof and (b) an antigen selected from AFP, APRIL, BCMA, CD123/IL3Ra, CD133, CD135/FLT3, CD138, CD147, CD19, CD20, CD22, CD239 (BCAM), CD276 (B7-H3), CD30, CD314/NKG2D, CD319/CS1/SLAMF7, CD326/EPCAM/TROP1, CD37, CD38, CD44v6, CD5, CD7, CD70, CLDN18.2, CLDN6, cMET, EGFRvIII, EPHA2, FAP, FR alpha, GD2, GPC3, IL13Ralpha2, Integrin B7, Lewis Y (LeY), MESO, MG7 antigen, MUC1, NECTIN4, NKG2DL, PSCA, PSMA/FOL1, ROBO1, ROR1, ROR2, TNFRSF13B/TACI, TRBC1, TRBC2, and TROP 2, as well as any variant thereof.

In embodiments, the antigen binding region binds two antigen, the antigens being: (a) an antigen selected from CD16, CD64, CD78, CD96, CLL1, CD116, CD117, CD71, CD45, CD71, CD123 and CD138, a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, hsp70-2, M-CSF, prostase, prostase specific antigen (PSA), PAP, NY-ESO-1, LAGA-1a, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-1, IGF-I I, IGFI receptor, mesothelin, a major histocompatibility complex (MHC) molecule presenting a tumor-specific peptide epitope, 5T4, ROR1, Nkp30, N KG2D, tumor stromal antigens, the extra domain A (EDA) and extra domain B (EDB) of fibronectin and the Al domain of tenascin-C (TnC Al) and fibroblast associated protein (FAP); a lineage-specific or tissue specific antigen such as CD3, CD4, CD8, CD24, CD25, CD33, CD34, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF17), multiple myeloma or lymphoblastic leukemia antigen, such as one selected from TNFRSF17, SLAMF7, GPRC5D, FKBP11, KAMP3, ITGA8, and FCRL5, a virus-specific surface antigen such as an HIV-specific antigen (such as HIV gpl20); an EBV-specific antigen, a CMV-specific antigen, a HPV-specific antigen, a Lasse Virus-specific antigen, an Influenza Virus-specific antigen, as well as any variant thereof and (b) an antigen selected from CD16, CD64, CD78, CD96, CLL1, CD116, CD117, CD71, CD45, CD71, CD123 and CD138, a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD30, CD40, disialoganglioside GD2, ductal-epithelial mucine, gp36, TAG-72, glycosphingolipids, glioma-associated antigen, β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, CD22, insulin growth factor (IGF1)-1, IGF-I I, IGFI receptor, mesothelin, a major histocompatibility complex (MHC) molecule presenting a tumor-specific peptide epitope, 5T4, ROR1, Nkp30, N KG2D, tumor stromal antigens, the extra domain A (EDA) and extra domain B (EDB) of fibronectin and the Al domain of tenascin-C (TnC Al) and fibroblast associated protein (FAP); a lineage-specific or tissue specific antigen such as CD3, CD4, CD8, CD24, CD25, CD33, CD34, CD133, CD138, CTLA-4, B7-1 (CD80), B7-2 (CD86), GM-CSF, cytokine receptors, endoglin, a major histocompatibility complex (MHC) molecule, BCMA (CD269, TNFRSF 17), multiple myeloma or lymphoblastic leukemia antigen, such as one selected from TNFRSF17, SLAMF7, GPRC5D, FKBP11, KAMP3, ITGA8, and FCRL5, a virus-specific surface antigen such as an HIV-specific antigen (such as HIV gpl20); an EBV-specific antigen, a CMV-specific antigen, a HPV-specific antigen, a Lasse Virus-specific antigen, an Influenza Virus-specific antigen, as well as any variant thereof.

In embodiments, the extracellular domain of the recombinant CAR comprises the extracellular domain of an NK cell activating receptor or a scFv.

In embodiments, the NK cell comprises a gene-edit in one or more of IL-7, CCL17, CCR4, IL-6, IL-6R, IL-12, IL-15, NKG2A, NKG2D, KIR, TRAIL, TRAC, PD1, and HPK1.

In embodiments, the gene-edit in one or more of IL-7, CCL17, CCR4, IL-6, IL-6R, IL-12, IL-15, NKG2A, NKG2D, KIR, TRAIL, TRAC, PD1, and HPK1 is caused by contacting the cell with RNA encoding one or more gene-editing proteins. In embodiments, the gene-edit of causes a reduction or elimination of expression and/or activity of IL-6, NKG2A, NKG2D, KIR, TRAC, PD1, and/or HPK1. In embodiments, the gene-edit causes an increase of expression and/or activity of IL-7, CCL17, CCR4, IL-6R, IL-12, IL-15, and/or TRAIL.

In embodiments, the cytotoxic lymphocyte, e.g., a T cell, NK cell, further comprises one or more recombinant genes capable of encoding a suicide gene product. In embodiments, the suicide gene product comprises a protein selected from the group consisting of thymidine kinase and an apoptotic signaling protein.

Any cytotoxic lymphocyte disclosed herein (e.g., manufactured by a method disclosed herein, comprising a gene edit (e.g., in B2M), expressing a high affinity CD16a receptor, and/or expressing a fusion protein comprising B2M polypeptide and an HLA polypeptide) can be further genetically engineered to express a CAR.

### Reprogramming Methods

In embodiments, the present disclosure relates to RNA-based modifications, e.g., reprogramming and/or gene-editing. In some embodiments, an RNA molecule encodes a gene-editing protein. In some embodiments, an RNA molecule encodes a reprogramming factor.

In embodiments, the RNA is mRNA. In embodiments, the RNA is modified mRNA. In embodiments, the modified mRNA comprises one or more non-canonical nucleotides.

In various embodiments, the present invention relates to the reprogramming of iPSCs to Monocytes, which can then be further differentiated into M1 and/or M2 macrophages, using non-viral, RNA-based means. iPSCs, namely pluripotent or less differentiated cells, can be reprogrammed from non-pluripotent or differentiated cells, including fibroblasts, keratinocytes, melanocyte blood cells, bone marrow cells, adipose cells, and tissue-resident progenitor cells.

In some embodiments, the method for reprogramming a non-pluripotent cell comprises: (a) providing a non-pluripotent cell; (b) culturing the non-pluripotent cell; and (c) transfecting the non-pluripotent cell with one or more synthetic RNA molecules, wherein the one or more synthetic RNA molecules include at least one RNA molecule encoding one or more reprogramming factors selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, wherein the transfecting results in the cell expressing the one or more reprogramming factors to result in the cell being reprogrammed; and wherein step (c) occurs in the presence of a medium containing ingredients that support reprogramming of the differentiated cell to a less differentiated state.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, wherein the transfecting results in the cell expressing the one or more reprogramming factors to result in the cell being reprogrammed to a less differentiated state; and wherein step (c) occurs in the presence of a medium containing ingredients that support reprogramming of the differentiated cell to a less differentiated state.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors; wherein the transfecting results in the cell expressing the one or more reprogramming factors; and wherein step (c) is performed at least twice and the amount of one or more synthetic RNA molecules transfected in one or more later transfections is greater than the amount transfected in one or more earlier transfections to result in the cell being reprogrammed to a less differentiated state and occurs in the presence of a medium containing ingredients that support reprogramming of the differentiated cell to a less differentiated state.

In some embodiments, the method for reprogramming a non-pluripotent cell, comprises: (a) providing a non-pluripotent cell; (b) culturing the non-pluripotent cell; and (c) transfecting the non-pluripotent cell with one or more synthetic RNA molecules, wherein the one or more synthetic RNA molecules include at least one RNA molecule encoding one or more reprogramming factors; wherein the transfecting results in the cell expressing the one or more reprogramming factors to result in the cell being reprogrammed; and wherein step (c) is performed without using irradiated human neonatal fibroblast feeder cells and occurs in the presence of a medium containing ingredients that support reprogramming of the cell.

In some embodiments, the method for reprogramming a differentiated cell to a less differentiated state, comprises: (a) providing a differentiated cell; (b) culturing the differentiated cell; and (c) transfecting the differentiated cell with one or more synthetic RNA molecules, wherein the one or more synthetic RNA molecules include at least one RNA molecule encoding one or more reprogramming factors; wherein the transfecting results in the cell expressing the one or more reprogramming factors to result in the cell being reprogrammed to a less differentiated state; and wherein step (c) is performed without using irradiated human neonatal fibroblast feeder cells and occurs in the presence of a medium containing ingredients that support reprogramming of the cell to a less differentiated state.

In some embodiments, the method for reprogramming a non-pluripotent cell, comprises: (a) providing a non-pluripotent cell; (b) culturing the non-pluripotent cell; (c) transfecting the non-pluripotent cell with one or more synthetic RNA molecules, wherein the one or more synthetic RNA molecules include at least one RNA molecule encoding one or more reprogramming factors and wherein the transfecting results in the cell expressing the one or more reprogramming factors; and (d) repeating step (c) at least twice during 5 consecutive days, wherein the amount of one or more synthetic RNA molecules transfected in one or more later transfections is greater than the amount transfected in one or more earlier transfections, to result in the non-pluripotent cell being reprogrammed, wherein steps (c) and (d) occur in the presence of a medium containing ingredients that support reprogramming of the non-pluripotent cell.

In some embodiments, the method for reprogramming a differentiated cell to a less differentiated state, comprises: (a) providing a differentiated cell; (b) culturing the differentiated cell; (c) transfecting the differentiated cell with one or more synthetic RNA molecules, wherein the one or more synthetic RNA molecules include at least one RNA molecule encoding one or more reprogramming factors and wherein the transfecting results in the cell expressing the one or more reprogramming factors; and (d) repeating step (c) at least twice during 5 consecutive days, wherein the amount of one or more synthetic RNA molecules transfected in one or more later transfections is greater than the amount transfected in one or more earlier transfections, to result in the cell being reprogrammed to a less differentiated state, wherein steps (c) and (d) occur in the presence of a medium containing ingredients that support reprogramming of the differentiated cell to a less differentiated state.

In some embodiments, the method for reprogramming a non-pluripotent cell comprises: (a) providing a non-pluripotent cell, the non-pluripotent cell being derived from a biopsy of a human subject; (b) culturing the non-pluripotent cell; and (c) transfecting the non-pluripotent cell with a synthetic RNA molecule, wherein: the synthetic RNA molecule encodes one or more reprogramming factor(s) selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, the transfecting results in the non-pluripotent cell expressing the one or more reprogramming factor(s) which reprograms the non-pluripotent cell; and step (c) is performed without using irradiated human neonatal fibroblast feeder cells and occurs in the presence of a medium containing ingredients that support reprogramming of the non-pluripotent cell.

In some embodiments, the method for reprogramming a cell to a less differentiated state, comprises: (a) providing a non-pluripotent cell; (b) culturing the cell; and (c) transfecting the cell with a synthetic RNA molecule, wherein: the RNA molecule encodes one or more reprogramming factor(s) selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, the transfecting results in the cell expressing the one or more reprogramming factor(s) which reprograms the cell to a less differentiated state, and step (c) is performed without using irradiated human neonatal fibroblast feeder cells and occurs in the presence of a medium containing ingredients that support reprogramming of the cell to a less differentiated state.

In some embodiments, the method for reprogramming a cell to a less differentiated state, comprises: (a) providing a non-pluripotent cell; (b) culturing the cell in a medium containing ingredients that support reprogramming of the cell to a less differentiated state; and (c) transfecting the cell with a synthetic RNA molecule, wherein: the RNA molecule encodes one or more reprogramming factor(s) selected from the group consisting of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, the transfecting results in the cell expressing the one or more reprogramming factor(s) which reprograms the cell to a less differentiated state, and step (c) is performed without using irradiated human neonatal fibroblast feeder cells and occurs in the presence of a feeder cell conditioned medium.

In some embodiments, the method for reprogramming a cell to a less differentiated state comprises: (a) providing a non-pluripotent cell; (b) culturing the cell in a medium containing albumin and ingredients that support reprogramming of the cell to a less differentiated state, wherein the albumin is treated with an ion-exchange resin or charcoal; (c) transfecting the cell with a synthetic RNA molecule, wherein the RNA molecule encoding one or more reprogramming factor(s) selected from the group consisting of Oct4 protein, Sox2 protein, Klf4 protein, c-Myc protein, l-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof, wherein the transfecting results in the cell expressing the one or more reprogramming factor(s) which reprograms the cell to a less differentiated state.

In some embodiments, the method for reprogramming a cell to a less differentiated state, comprises: (a) culturing a differentiated cell with a reprogramming medium; (b) transfecting the cell with one or more synthetic RNA molecules, wherein the one or more synthetic RNA molecules include at least one RNA molecule encoding one or more reprogramming factors and wherein the transfecting results in the cell expressing the one or more reprogramming factors; and (c) repeating step (b) at least twice during 5 consecutive days, wherein the amount of one or more synthetic RNA molecules transfected in one or more later transfections is greater than the amount transfected in one or more earlier transfections, to result in the cell being reprogrammed to a less differentiated state, wherein steps (a)-(c) are performed without using feeder cells and occur in the presence of a feeder cell conditioned medium.

In some embodiments, the method for reprogramming a cell to a less differentiated state, comprises: a. culturing a differentiated cell with a reprogramming medium containing albumin, wherein the albumin is treated with an ion-exchange resin or charcoal; b. transfecting the cell with one or more synthetic RNA molecules, wherein the one or more synthetic RNA molecules includes at least one RNA molecule encoding one or more reprogramming factors and wherein the transfecting results in the cell expressing the one or more reprogramming factors; and c. repeating step (b) at least twice during 5 consecutive days to result in the cell being reprogrammed to a less differentiated state.

In some embodiments, the method for reprogramming a cell to a less differentiated state, comprises: a. culturing a differentiated cell with a reprogramming medium containing albumin, wherein the albumin is treated with sodium octanoate; brought to a temperature of at least about 40° C.; and treated with an ion-exchange resin or charcoal; b. transfecting the cell with one or more synthetic RNA molecules, wherein the one or more synthetic RNA molecules includes at least one RNA molecule encoding one or more reprogramming transcription factors and wherein the transfecting results in the cell expressing the one or more synthetic RNA molecules; and c. repeating step (b) at least twice during about 5 consecutive days to result in the cell being reprogrammed to a less differentiated state.

In embodiments, the reprogramming is non-viral. In embodiments, the reprogramming factor is one or more of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

In some embodiments, iPSCs are obtained and Monocytes, which can then be further differentiated into M1 and/or M2 macrophages, are generated via cell reprogramming with non-immunogenic messenger RNA (mRNA) encoding one or more reprogramming factors in a defined, animal component-free process. In some embodiments the process is immunosuppressant-free. In some embodiments, the process is animal component-free. In some embodiments, the process is defined.

In some embodiments, iPSCs are generated from adult human dermal fibroblasts using a high-efficiency, immunosuppressant-free mRNA-based protocol, whereupon iPSCs are differentiated into monocytes using a 28-day monolayer protocol. Beginning on day 14, cells can be harvested every 3-4 days. CD14+ isolation yielded >95% CD14+ cells with an average yield of 4.1×10⁴ cells per cm2 per harvest. iPSC-derived monocytes were compared to peripheral blood mononuclear cell (PBMC)-derived monocytes for expression of key hematopoietic and myeloid-lineage markers CD11b, CD14, CD33, CD45, CD80, CD163, CD206, and SIRPα. In some embodiments, the differentiated monocytes are characterized by downregulation of Nanog and Oct4 and/or changes in expression of CD11b, CD14, CD33, CD45, CD80, CD163, CD206, and SIRPα, e.g., relative to the source cells. In some embodiments, rtPCR analysis is used to characterize monocytes. In some embodiments, monocytes are further differentiated into M1 and/or M2 macrophages. iPSC-derived monocytes can be further differentiated into macrophages by exposure to MCSF for 3-4 days. The macrophages can be assessed for their ability to polarize, secrete pro- and anti-inflammatory cytokines, and for cytotoxic activity when co-cultured with cancer cells. M1 macrophages can be polarized with interferon gamma (IFN-γ, 50 ng/mL) and lipopolysaccharide (LPS, 10 ng/mL) for 48 hours, whereas M2 macrophages can be treated with IL-4 (10 ng/mL) for 48 hours. The efficiency of iPSC-derived monocytes differentiation into macrophages can be assessed by cell adherence, morphology, and surface marker expression (CD14, CD45, CD163). The ability of M1 and M2 polarized iPSC-derived macrophages to secrete TNFα, IL-12p70, and IL-10 can be assayed and compared to PBMC-derived macrophages. Finally, the ability of M1 and M2 polarized iPSC-derived macrophages to kill cancer cells can be assayed.

Cells can be reprogrammed by exposing them to specific extracellular cues and/or by ectopic expression of specific proteins, microRNAs, etc. While several reprogramming methods have been previously described, most that rely on ectopic expression require the introduction of exogenous DNA, which can carry mutation risks. DNA-free reprogramming methods based on direct delivery of reprogramming proteins have been reported. However, these methods are too inefficient and unreliable for commercial use. In addition, RNA-based reprogramming methods have been described (see, e.g., Angel. MIT Thesis. 2008. 1-56; Angel et al. PLoS ONE. 2010. 5,107; Warren et al. Cell Stem Cell. 2010. 7,618-630; Angel. MIT Thesis. 2011. 1-89; and Lee et al., Cell. 2012. 151,547-558; the contents of all of which are hereby incorporated by reference). However, existing RNA-based reprogramming methods are slow, unreliable, and inefficient when performed on adult cells, require many transfections (resulting in significant expense and opportunity for error), can reprogram only a limited number of cell types, can reprogram cells to only a limited number of cell types, require the use of immunosuppressants, and require the use of multiple human-derived components, including blood-derived HSA and human fibroblast feeders. The many drawbacks of previously disclosed RNA-based reprogramming methods make them undesirable for research, therapeutic or cosmetic use.

In some embodiments, reprogramming is performed by transfecting cells with one or more nucleic acids encoding one or more reprogramming factors, including, but not limited to Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In one embodiment, the cell is a human skin cell, and the human skin cell is reprogrammed to a pluripotent stem cell. In another embodiment, the cell is a human skin cell, and the human skin cell is reprogrammed to a glucose-responsive insulin-producing cell. Examples of other cells that can be reprogrammed and other cells to which a cell can be reprogrammed include, but are not limited to skin cells, pluripotent stem cells, MSCs, β-cells, retinal pigmented epithelial cells, hematopoietic cells, cardiac cells, airway epithelial cells, neural stem cells, neurons, glial cells, bone cells, blood cells, and dental pulp stem cells. In one embodiment, the cell is contacted with a medium that supports the reprogrammed cell. In one embodiment, the medium also supports the cell.

Importantly, infecting skin cells with viruses encoding Oct4, Sox2, Klf4, and c-Myc, combined with culturing the cells in a medium that supports the growth of cardiomyocytes, has been reported to cause reprogramming of the skin cells to cardiomyocytes, without first reprogramming the skin cells to pluripotent stem cells (See Efs et al Nat Cell Biol. 2011; 13:215-22, the contents of which are hereby incorporated by reference). In certain situations, direct reprogramming (reprogramming one somatic cell to another somatic cell without first reprogramming the somatic cell to a pluripotent stem cell, also known as “transdifferentiation”) may be desirable, in part because culturing pluripotent stem cells can be time-consuming and expensive, the additional handling involved in establishing and characterizing a stable pluripotent stem cell line can carry an increased risk of contamination, and the additional time in culture associated with first producing pluripotent stem cells can carry an increased risk of genomic instability and the acquisition of mutations, including point mutations, copy-number variations, and karyotypic abnormalities.

In embodiments, fewer total transfections may be required to reprogram a cell according to the methods of the present invention than according to other methods. Certain embodiments are therefore directed to a method for reprogramming a cell, wherein from about 1 to about 12 transfections are performed during about 20 consecutive days, or from about 4 to about 10 transfections are performed during about 15 consecutive days, or from about 4 to about 8 transfections are performed during about 10 consecutive days. It is recognized that when a cell is contacted with a medium containing nucleic acid molecules, the cell may likely come into contact with and/or internalize more than one nucleic acid molecule either simultaneously or at different times. A cell can therefore be contacted with a nucleic acid more than once, e.g., repeatedly, even when a cell is contacted only once with a medium containing nucleic acids.

Of note, nucleic acids can contain one or more non-canonical or “modified” residues as described herein. For instance, any of the non-canonical nucleotides described herein can be used in the present reprogramming methods. In one embodiment, pseudouridine-5′-triphosphate can be substituted for uridine-5′-triphosphate in an in vitro-transcription reaction to yield synthetic RNA, wherein up to 100% of the uridine residues of the synthetic RNA may be replaced with pseudouridine residues. In vitro-transcription can yield RNA with residual immunogenicity, even when pseudouridine and 5-methylcytidine are completely substituted for uridine and cytidine, respectively (see, e.g., Angel. Reprogramming Human Somatic Cells to Pluripotency Using RNA [Doctoral Thesis]. Cambridge, MA: MIT; 2011, the contents of which are hereby incorporated by reference). For this reason, it is common to add an immunosuppressant to the transfection medium when transfecting cells with RNA. In certain situations, adding an immunosuppressant to the transfection medium may not be desirable, in part because the recombinant immunosuppressant most commonly used for this purpose, B18R, can be expensive and difficult to manufacture. In one embodiment, the immunosuppressant is B18R or a biologically active fragment, analogue, variant or family-member thereof or dexamethasone or a derivative thereof. In one embodiment, the transfection medium does not contain an immunosuppressant, and the nucleic-acid dose is chosen to prevent excessive toxicity. In another embodiment, the nucleic-acid dose is less than about 1 mg/cm² of tissue or less than about 1 mg/100,000 cells or less than about 10 mg/kg.

In various cases, transfection of a cell with synthetic nucleic acids for reprogramming the cell may be facilitated by use of the ToRNAdo™ Nucleic-Acid Delivery System. This system relates to new lipids that find use, inter alia, in improved delivery of biological payloads, e.g., nucleic acids, to cells. The system relates to use of a compound of Formula (IV):

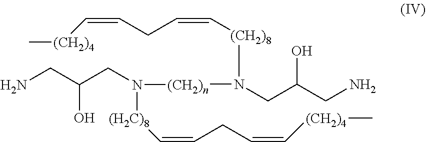

where n is 1, 2, 3, 4, 5, 6, 7, 8, 9, 10, 11, 12, 13, 14, or 15. Further description of ToRNAdo™ Nucleic-Acid Delivery System is found in one or both of U.S. Pat. No. 10,501,404 and US20210009505A1. The entire contents of which are incorporated by reference in their entirety.

In any of the herein-disclosed aspects or embodiments, a synthetic RNA molecule may be in the form of a circular RNA (circRNA). The circRNA are manufactured by methods do not require a linear oligonucleotide (splint) to pre-orient the two reacting ends of a linear RNA to assist in ligation to yield a circRNA, the circRNA are manufactured by methods that do not require ribozymes to yield a circRNA, and/or the circRNA are manufactured by methods that do not require HPLC-based purification, e.g., post-ligation. A nucleic acid that can be manufactured into a circRNA has the structure: 5′-X-Y-A-IRES-B-CDS-C-Y′-Z 3′. Here, Y and Y′ each independently comprise one or more nucleotides and Y and Y′ are substantially complementary; X and Z each independently comprise one or more nucleotides and X and Z are not substantially complementary; IRES comprises an internal ribosome entry site; CDS comprises a coding sequence; and A, B, and C are each independently a spacer comprising one or more nucleotides or null. The CDS of a circRNA may encode one or more proteins of interest, the protein of interest being one or more reprogramming factors, optionally selected from Oct4, Sox2, Klf4, c-Myc, 1-Myc, Tert, Nanog, Lin28, Glis1, Utf1, Aicda, miR200 micro-RNA, miR291 micro-RNA, miR294 micro-RNA and miR295 micro-RNA, miR302 micro-RNA, miR367 micro-RNA, miR369 micro-RNA, or a natural or engineered variant, family member, orthologue, fragment or fusion construct thereof. In some cases, the CDS encodes two, three, four, five, six, seven, eight, nine, ten, eleven, or more reprogramming factor(s). Additional details regarding circRNAs useful in the present disclosure are described in PCT/US2022/026564, the contents of which are incorporated herein by reference in its entirety.

Reprogrammed cells produced according to certain embodiments of the present invention are suitable for therapeutic and/or cosmetic applications as they do not contain undesirable exogenous DNA sequences, and they are not exposed to animal-derived or human-derived products, which may be undefined, and which may contain toxic and/or pathogenic contaminants. Furthermore, the high speed, efficiency, and reliability of certain embodiments of the present invention may reduce the risk of acquisition and accumulation of mutations and other chromosomal abnormalities. Certain embodiments of the present invention can thus be used to generate cells that have a safety profile adequate for use in therapeutic and/or cosmetic applications. For example, reprogramming cells using RNA and the medium of the present invention, wherein the medium does not contain animal or human-derived components, can yield cells that have not been exposed to allogeneic material. Certain embodiments are therefore directed to a reprogrammed cell that has a desirable safety profile. In one embodiment, the reprogrammed cell has a normal karyotype. In another embodiment, the reprogrammed cell has fewer than about 5 copy-number variations (CNVs) relative to the patient genome, such as fewer than about 3 copy-number variations relative to the patient genome, or no copy-number variations relative to the patient genome. In yet another embodiment, the reprogrammed cell has a normal karyotype and fewer than about 100 single nucleotide variants in coding regions relative to the patient genome, or fewer than about 50 single nucleotide variants in coding regions relative to the patient genome, or fewer than about 10 single nucleotide variants in coding regions relative to the patient genome.

Endotoxins and nucleases can co-purify and/or become associated with other proteins, such as serum albumin. Recombinant proteins, in particular, can often have high levels of associated endotoxins and nucleases, due in part to the lysis of cells that can take place during their production. Endotoxins and nucleases can be reduced, removed, replaced or otherwise inactivated by many of the methods of the present invention, including, for example, by acetylation, by addition of a stabilizer such as sodium octanoate, followed by heat treatment, by the addition of nuclease inhibitors to the albumin solution and/or medium, by crystallization, by contacting with one or more ion-exchange resins, by contacting with charcoal, by preparative electrophoresis or by affinity chromatography. In embodiments, partially or completely reducing, removing, replacing, or otherwise inactivating endotoxins and/or nucleases from a medium and/or from one or more components of a medium is provided and this can increase the efficiency with which cells can be transfected and reprogrammed. Certain embodiments are therefore directed to a method for transfecting a cell with one or more nucleic acids, wherein the transfection medium is treated to partially or completely reduce, remove, replace or otherwise inactivate one or more endotoxins and/or nucleases. Other embodiments are directed to a medium that causes minimal degradation of nucleic acids. In one embodiment, the medium contains less than about 1 EU/mL, or less than about 0.1 EU/mL, or less than about 0.01 EU/mL.

In certain situations, protein-based lipid carriers such as serum albumin can be replaced with non-protein-based lipid carriers such as methyl-beta-cyclodextrin. The medium of the present invention can also be used without a lipid carrier, for example, when transfection is performed using a method that may not require or may not benefit from the presence of a lipid carrier, for example, using one or more lipid-based transfection reagents, polymer-based transfection reagents or peptide-based transfection reagents or using electroporation. Many protein-associated molecules, such as metals, can be highly toxic to cells in vivo. This toxicity can cause decreased viability, as well as the acquisition of mutations. Certain embodiments thus have the additional benefit of producing cells that are free from toxic molecules.

The associated-molecule component of a protein can be measured by suspending the protein in solution and measuring the conductivity of the solution. Certain embodiments are therefore directed to a medium that contains a protein, wherein about a 10% solution of the protein in water has a conductivity of less than about 500 μmho/cm. In one embodiment, the solution has a conductivity of less than about 50 μmho/cm. In another embodiment, less than about 0.65% of the dry weight of the protein comprises lipids and/or less than about 0.35% of the dry weight of the protein comprises free fatty acids.

Certain embodiments are therefore directed to a method for transfecting a cell with a nucleic acid, wherein the cell is transfected more than once, and wherein the amount of nucleic acid delivered to the cell is different for two of the transfections. In one embodiment, the cell proliferates between two of the transfections, and the amount of nucleic acid delivered to the cell is greater for the second of the two transfections than for the first of the two transfections. In another embodiment, the cell is transfected more than twice, and the amount of nucleic acid delivered to the cell is greater for the second of three transfections than for the first of the same three transfections, and the amount of nucleic acid delivered to the cells is greater for the third of the same three transfections than for the second of the same three transfections. In yet another embodiment, the cell is transfected more than once, and the maximum amount of nucleic acid delivered to the cell during each transfection is sufficiently low to yield at least about 80% viability for at least two consecutive transfections.

In embodiments, there are provided methods in which modulating the amount of nucleic acid delivered to a population of proliferating cells in a series of transfections can result in both an increased effect of the nucleic acid and increased viability of the cells. In embodiments, when cells are contacted with one or more nucleic acids encoding one or more reprogramming factors in a series of transfections, the efficiency of reprogramming can be increased when the amount of nucleic acid delivered in later transfections is greater than the amount of nucleic acid delivered in earlier transfections, for at least part of the series of transfections. Certain embodiments are therefore directed to a method for reprogramming a cell, wherein one or more nucleic acids is repeatedly delivered to the cell in a series of transfections, and the amount of the nucleic acid delivered to the cell is greater for at least one later transfection than for at least one earlier transfection. In one embodiment, the cell is transfected from about 2 to about 10 times, or from about 3 to about 8 times, or from about 4 to about 6 times. In another embodiment, the one or more nucleic acids includes at least one RNA molecule, the cell is transfected from about 2 to about 10 times, and the amount of nucleic acid delivered to the cell in each transfection is the same as or greater than the amount of nucleic acid delivered to the cell in the most recent previous transfection. In yet another embodiment, the amount of nucleic acid delivered to the cell in the first transfection is from about 20 ng/cm² to about 250 ng/cm², or from 100 ng/cm² to 600 ng/cm². In yet another embodiment, the cell is transfected about 5 times at intervals of from about 12 to about 48 hours, and the amount of nucleic acid delivered to the cell is about 25 ng/cm² for the first transfection, about 50 ng/cm² for the second transfection, about 100 ng/cm² for the third transfection, about 200 ng/cm² for the fourth transfection, and about 400 ng/cm² for the fifth transfection. In yet another embodiment, the cell is further transfected at least once after the fifth transfection, and the amount of nucleic acid delivered to the cell is about 400 ng/cm².

Several molecules can be added to media by conditioning. Certain embodiments are therefore directed to a medium that is supplemented with one or more molecules that are present in a conditioned medium. In one embodiment, the medium is supplemented with Wnt1, Wnt2, Wnt3, Wnt3a or a biologically active fragment, analogue, variant, agonist, or family-member thereof. In another embodiment, the medium is supplemented with TGF-β or a biologically active fragment, analogue, variant, agonist, or family-member thereof. In yet another embodiment, a cell is reprogrammed according to the method of the present invention, wherein the medium is not supplemented with TGF-β for from about 1 to about 5 days and is then supplemented with TGF-β for at least about 2 days. In yet another embodiment, the medium is supplemented with IL-6, IL-6R or a biologically active fragment, analogue, variant, agonist, or family-member thereof. In yet another embodiment, the medium is supplemented with a sphingolipid or a fatty acid. In still another embodiment, the sphingolipid is lysophosphatidic acid, lysosphingomyelin, sphingosine-1-phosphate or a biologically active analogue, variant or derivative thereof.

In addition to mitotically inactivating cells, under certain conditions, irradiation can change the gene expression of cells, causing cells to produce less of certain proteins and more of certain other proteins than non-irradiated cells, for example, members of the Wnt family of proteins. In addition, certain members of the Wnt family of proteins can promote the growth and transformation of cells. In embodiments, the efficiency of reprogramming can be greatly increased by contacting a cell with a medium that is conditioned using irradiated feeders instead of mitomycin-c-treated feeders. In embodiments, the increase in reprogramming efficiency observed when using irradiated feeders is caused in part by Wnt proteins that are secreted by the feeders. Certain embodiments are therefore directed to a method for reprogramming a cell, wherein the cell is contacted with Wnt1, Wnt2, Wnt3, Wnt3a or a biologically active fragment, analogue, variant, family-member or agonist thereof, including agonists of downstream targets of Wnt proteins, and/or agents that mimic one or more of the biological effects of Wnt proteins, for example, 2-amino-4-[3,4-(methylenedioxy)benzylamino]-6-(3-methoxyphenyl)pyrimidine.

Because of the low efficiency of many previously described DNA-based reprogramming methods, these methods may be difficult or impossible to use with cells derived from patient samples, which may contain only a small number of cells. In contrast, the high efficiency of certain embodiments of the present invention can allow reliable reprogramming of a small number of cells, including single cells. Certain embodiments are directed to a method for reprogramming a small number of cells. Other embodiments are directed to a method for reprogramming a single cell. In one embodiment, the cell is contacted with one or more enzymes. In another embodiment, the enzyme is collagenase. In yet another embodiment, the collagenase is animal-component free. In one embodiment, the collagenase is present at a concentration of from about 0.1 mg/mL to about 10 mg/mL, or from about 0.5 mg/mL to about 5 mg/mL. In another embodiment, the cell is a blood cell. In yet another embodiment, the cell is contacted with a medium containing one or more proteins that is derived from the patient's blood. In still another embodiment, the cell is contacted with a medium comprising: DMEM/F12+2 mM L-alanyl-L-glutamine+from about 5% to about 25% patient-derived serum, or from about 10% to about 20% patient-derived serum, or about 20% patient-derived serum.

In embodiments, transfecting cells with a mixture of RNA encoding Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof using the medium of the present invention can cause the rate of proliferation of the cells to increase. When the amount of RNA delivered to the cells is too low to ensure that all of the cells are transfected, only a fraction of the cells may show an increased proliferation rate. In certain situations, such as when generating a personalized therapeutic, increasing the proliferation rate of cells may be desirable, in part because doing so can reduce the time necessary to generate the therapeutic, and therefore can reduce the cost of the therapeutic. Certain embodiments are therefore directed to a method for transfecting a cell with a mixture of RNA encoding Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In one embodiment, the cell exhibits an increased proliferation rate. In another embodiment, the cell is reprogrammed.

While detailed examples are provided herein for the production of specific types of cells and for the production of therapeutics comprising specific types of cells, it is recognized that the methods of the present invention can be used to produce many other types of cells, and to produce therapeutics comprising one or more of many other types of cells, for example, by reprogramming a cell according to the methods of the present invention, and culturing the cell under conditions that mimic one or more aspects of development by providing conditions that resemble the conditions present in the cellular microenvironment during development.

Other embodiments are directed to a method for reprogramming a cell. In one embodiment, the cell is reprogrammed by contacting the cell with one or more nucleic acids. In one embodiment, the cell is contacted with a plurality of nucleic acids encoding at least one of Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof. In another embodiment, the cell is contacted with a plurality of nucleic acids encoding a plurality of proteins including: Oct4 protein, Sox2 protein, Klf4 protein, c-Myc protein, 1-Myc protein, TERT protein, Nanog protein, Lin28 protein, Glis1 protein, Utf1 protein, Aicda protein, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family-members thereof.

Illustrative subjects or patients refers to any vertebrate including, without limitation, humans and other primates (e.g., chimpanzees and other apes and monkey species), farm animals (e.g., cattle, sheep, pigs, goats, and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In some embodiments, the subject is a human.

In some embodiments, a synthetic RNA molecule is used to reprogram iPSCs into monocytes, which can then be further differentiated into M1 and/or M2 macrophages. In embodiments, the synthetic RNA molecule is mRNA. In embodiments, the synthetic RNA molecule is in vitro transcribed. In embodiments, the synthetic RNA is a circRNA.

In embodiments, the RNA is mRNA. In embodiments, the RNA is modified mRNA. In embodiments, the modified mRNA comprises one or more non-canonical nucleotides. In some embodiments, non-canonical nucleotides are incorporated into RNA to increase the efficiency with which the RNA can be translated into protein, and can decrease the toxicity of the RNA. In embodiments, the RNA molecule comprises one or more non-canonical nucleotides. In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 5, about 10, about 15, about 20 nucleotides, about 30, about 40, about 50, about 60, about 70, about 80, about 90, about 100, about 125, about 150, about 175, about 200, about 225, about 250, about 275, about 300, about 400 to about 500 nucleotides.

Certain embodiments are directed to a nucleic acid comprising a 5′-cap structure selected from Cap 0, Cap 1, Cap 2, and Cap 3 or a derivative thereof. In one embodiment, the nucleic acid comprises one or more UTRs. In another embodiment, the one or more UTRs increase the stability of the nucleic acid. In a further embodiment, the one or more UTRs comprise an alpha-globin or beta-globin 5′-UTR. In a still further embodiment, the one or more UTRs comprise an alpha-globin or beta-globin 3′-UTR. In a still further embodiment, the RNA molecule comprises an alpha-globin or beta-globin 5′-UTR and an alpha-globin or beta-globin 3′-UTR. In one embodiment, the 5′-UTR comprises a Kozak sequence that is substantially similar to the Kozak consensus sequence. In another embodiment, the nucleic acid comprises a 3′-poly(A) tail. In a further embodiment, the 3′-poly(A) tail is from about 20nt to about 250nt or from about 120nt to about 150nt long. In a further embodiment, the 3′-poly(A) tail is about 5nt, or about 10nt, or about 15nt, or about 20nt, or about 30nt, or about 40nt, or about 50nt, or about 60nt, or about 70nt, or about 80nt, or about 90nt, or about 100nt, or about 110nt, or about 120nt, or about 130nt, or about 140nt, or about 150nt, or about 160nt, or about 170nt, or about 180nt, or about 190nt, or about 200nt, or about 210nt, or about 220nt, or about 230nt, or about 240nt, or about 250nt, or about 255nt, or about 260nt, or about 265nt, or about 270nt, or about 275nt, or about 280nt, or about 285, or about 290nt, or about 295nt, or about 300nt long.

In some embodiments, the RNA comprises a tail composed of a plurality of adenines with one or more guanines.

In embodiments, the RNA comprises (a) a sequence encoding a protein, and (b) a tail region comprising deoxyadenosine nucleotides and one or more other nucleotides.

In embodiments, the one or more other nucleotides comprises deoxyguanosine residues. In embodiments, the tail region comprises about 1%, about 2%, about 5%, about 10%, about 15%, about 20%, about 25%, about 30%, about 35%, about 40%, about 45%, or about 50% deoxyguanosine residues. In embodiments, the tail region comprises more than 50% deoxyguanosine residues.

In embodiments, the one or more other nucleotides comprises deoxycytidine residues. In embodiments, the tail region comprises about 1%, about 2%, about 5%, about 10%, about 15%, about 20%, about 25%, about 30%, about 35%, about 40%, about 45%, or about 50% deoxycytidine residues. In embodiments, the tail region comprises more than 50% deoxycytidine residues.

In embodiments, the one or more other nucleotides comprises deoxythymidine residues. In embodiments, the tail region comprises about 1%, about 2%, about 5%, about 10%, about 15%, about 20%, about 25%, about 30%, about 35%, about 40%, about 45%, or about 50% deoxythymidine residues. In embodiments, the tail region comprises more than 50% deoxythymidine residues.

In embodiments, the one or more other nucleotides comprise deoxyguanosine residues and deoxycytidine residues. In embodiments, the tail region comprises about 99%, about 98%, about 95%, about 90%, about 85%, about 80%, about 75%, about 70%, about 65%, about 60%, about 55%, or about 50% deoxyadenosine residues. In embodiments, the tail region comprises fewer than 50% deoxyadenosine residues.

In embodiments, the one or more other nucleotides comprises guanosine residues.

In embodiments, the tail region comprises about 1%, about 2%, about 5%, about 10%, about 15%, about 20%, about 25%, about 30%, about 35%, about 40%, about 45%, or about 50% guanosine residues. In embodiments, the tail region comprises more than 50% guanosine residues.

In embodiments, the one or more other nucleotides comprises cytidine residues. In embodiments, the tail region comprises about 1%, about 2%, about 5%, about 10%, about 15%, about 20%, about 25%, about 30%, about 35%, about 40%, about 45%, or about 50% cytidine residues. In embodiments, the tail region comprises more than 50% cytidine residues.

In embodiments, the one or more other nucleotides comprises uridine residues. In embodiments, the tail region comprises about 1%, about 2%, about 5%, about 10%, about 15%, about 20%, about 25%, about 30%, about 35%, about 40%, about 45%, or about 50% uridine residues. In embodiments, the tail region comprises more than 50% uridine residues.

In embodiments, the one or more other nucleotides comprise guanosine residues and cytidine residues. In embodiments, the tail region comprises about 99%, about 98%, about 95%, about 90%, about 85%, about 80%, about 75%, about 70%, about 65%, about 60%, about 55%, or about 50% adenosine residues.

In embodiments, the tail region comprises fewer than 50% adenosine residues.

In embodiments, the tail is (A)₁₅₀ (SEQ ID NO: 61). In embodiments, the tail is (A₃₉G)₃(A)₃₀ (SEQ ID NO: 62). In embodiments, the tail is (A₁₉G)₇(A)₁₀ (SEQ ID NO: 63). In embodiments, the tail is (A₉G)₁₅ (SEQ ID NO: 64).

In embodiments, the length of the tail region is between about 80 nucleotides and about 120 nucleotides, about 120 nucleotides and about 160 nucleotides, about 160 nucleotides and about 200 nucleotides, about 200 nucleotides and about 240 nucleotides, about 240 nucleotides and about 280 nucleotides, or about 280 nucleotides and about 320 nucleotides.

In embodiments, the length of the tail region is greater than 320 nucleotides.

In embodiments, the RNA comprises a 5′ cap structure. In embodiments, the RNA 5′-UTR comprises a Kozak consensus sequence. In embodiments, the RNA 5′-UTR comprises a sequence that increases RNA stability in vivo, and the 5′-UTR may comprise an alpha-globin or beta-globin 5′-UTR.

In embodiments, the RNA 3′-UTR comprises a sequence that increases RNA stability in vivo, and the 3′-UTR may comprise an alpha-globin or beta-globin 3′-UTR. In embodiments, the RNA comprises a 3′ poly(A) tail. In embodiments, the RNA 3′ poly(A) tail is from about 20 nucleotides to about 250 nucleotides in length.

In embodiments, the RNA is from about 200 nucleotides to about 5000 nucleotides in length.

In embodiments, the RNA is prepared by in vitro transcription. In embodiments, the RNA is synthetic.

In some embodiments, the synthetic RNA comprises about 200 nucleotides to about 5000 nucleotides. In some embodiments, the synthetic RNA comprises from about 500 to about 2000 nucleotides, or about 500 to about 1500 nucleotides, or about 500 to about 1000 nucleotides.

Further description of reprogramming is found in one or more of US20140356906A1, US20150267189A1, US20160324934A1, US20180256748A1, US20190008985A1, US20210024907A1, US20210009505A1, US20230193231A1, or US20230193207A1. The entire contents of which are incorporated by reference in their entirety.

### Gene-Editing

In any herein disclosed aspect or embodiment, a cell is gene-edited.

In some embodiments, gene-editing a cell comprises contacting the cell with a synthetic nucleic acid encoding one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a (Cpf1), Cas13a, Cas14, CasX, CasY, a Class 1 Cas protein, a Class 2 Cas protein, MAD7, and a gene-editing protein comprising a repeat sequence comprising LTPvQVVAIAwxyz (SEQ ID NO: 16), or a natural or engineered variant, family member, orthologue, fragment or fusion construct thereof.

In some embodiments, the gene-editing protein comprises first gene editing protein and/or the second gene editing protein comprises a plurality of repeat sequences and at least one of the repeat sequences comprises the amino acid sequence: LTPvQVVAIAwxyz (SEQ ID NO: 16), wherein, v is Q, D or E, w is S or N, x is I, H, N, or I, y is D, A, I, N, H, K, S, G or null, and z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20).

In some embodiments, the gene-editing protein comprises: (i) a DNA-binding domain comprising a plurality of repeat sequences and (ii) the nuclease domain comprising a catalytic domain of a nuclease. In embodiments, the at least one of the repeat sequences comprises the amino acid sequence: LTPvQVVAIAwxyzα (SEQ ID NO: 22) and is optionally between 36 and 39 amino acids long, where:
v is Q, D or E,
w is S or N,
x is I, H, N, or I,
y is D, A, I, N, H, K, S, G, or null,
z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), and
α is four consecutive amino acids.

In embodiments, α comprises at least one glycine (G) residue. In embodiments, α comprises at least one histidine (H) residue. In embodiments, α comprises at least one histidine (H) residue at any one of positions 33, 34, or 35. In embodiments, α comprises at least one aspartic acid (D) residue. In embodiments, α comprises at least one, or two, or three of a glycine (G) residue, a histidine (H) residue, and an aspartic acid (D) residue.

In some embodiments, α comprises one or more hydrophilic residues, optionally selected from: a polar and positively charged hydrophilic amino acid, optionally selected from arginine (R) and lysine (K); a polar and neutral of charge hydrophilic amino acid, optionally selected from asparagine (N), glutamine (Q), serine (S), threonine (T), proline (P), and cysteine (C); a polar and negatively charged hydrophilic amino acid, optionally selected from aspartate (D) and glutamate (E), and an aromatic, polar and positively charged hydrophilic amino acid, optionally selected from histidine (H).

In some embodiments, α comprises one or more polar and positively charged hydrophilic amino acids selected from arginine (R) and lysine (K). In some embodiments, α comprises one or more polar and neutral of charge hydrophilic amino acids selected from asparagine (N), glutamine (Q), serine (S), threonine (T), proline (P), and cysteine (C). In some embodiments, α comprises one or more polar and negatively charged hydrophilic amino acids selected from aspartate (D) and glutamate (E). In some embodiments, α comprises one or more aromatic, polar and positively charged hydrophilic amino acids selected from histidine (H).

In embodiments, α comprises one or more hydrophobic residues, optionally selected from: a hydrophobic, aliphatic amino acid, optionally selected from glycine (G), alanine (A), leucine (L), isoleucine (I), methionine (M), and valine (V), and a hydrophobic, aromatic amino acid, optionally selected from phenylalanine (F), tryptophan (W), and tyrosine (Y). In some embodiments, α comprises one or more hydrophobic, aliphatic amino acids selected from glycine (G), alanine (A), leucine (L), isoleucine (I), methionine (M), and valine (V). In some embodiments, α comprises one or more aromatic amino acids selected from phenylalanine (F), tryptophan (W), and tyrosine (Y). In embodiments, the DNA-binding domain comprises about 15, or about, 16, or about 17, or about 18, or about 18.5 repeat sequences.

In embodiments, a is selected from GHGG (SEQ ID NO: 31), HGSG (SEQ ID NO: 32), HGGG (SEQ ID NO: 33), from GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 4260

In embodiments, the gene-editing protein has a DNA binding domain having at least one repeat of LTPEQVVAIAS*RVD*GGKQALETVQRLLPVLCQAGHGG (SEQ ID NO: 65; the “*RVD*” corresponds to the dinucleotide “xy” of SEQ ID NO: 22).

In embodiments, the repeat sequence is 33 or 34 amino acids long. In embodiments, the repeat sequence is 36-39 amino acids long. In some embodiments, the repeat sequence is 36 amino acids long. In some embodiments, the repeat sequence is 37 amino acids long. In some embodiments, the repeat sequence is 38 amino acids long. In some embodiments, the repeat sequence is 39 amino acids long.

In embodiments, the gene-editing protein comprises (i) a DNA-binding domain comprising a plurality of repeat sequences and at least one of the repeat sequences comprises the amino acid sequence: LTPvQVVAIAwxyzGHGG (SEQ ID NO: 17) or LTPvQVVAIAwxyzGTHG (SEQ ID NO: 18) and is from 36 to 39 amino acids long, wherein: “v” is Q, D or E, “w” is S or N, “x” is H, N, or I, “y” is D, A, I, N, G, H, K, S, or null, and “z” is GGKQALETVQRLLPVLCQD (SEQ ID NO: 19) or GGKQALETVQRLLPVLCQA (SEQ ID NO: 20) and (ii) a nuclease domain comprising a catalytic domain of a nuclease. In some embodiments, a gene-editing protein comprises a C-terminal GTHG (SEQ ID NO: 21) produces more efficient editing at the target locus than TALENs at 33° C. GTHG (SEQ ID NO: 21). In various embodiments, a gene-editing protein comprises a C-terminal GTHG (SEQ ID NO: 21) produces more efficient editing at the target locus than TALENs at 37° C.

In embodiments, the gene-editing protein comprises (i) a DNA-binding domain comprising a plurality of repeat sequences and at least one of the repeat sequences comprises the amino acid sequence: LTPvQVVAIAwxyzα (SEQ ID NO: 22) and is from 36 to 39 amino acids long, wherein: v is Q, D or E, w is S or N, x is I, H, N, or I, y is D, A, I, N, H, K, S, G or null, z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), α is four consecutive amino acids; and (ii) a nuclease domain comprising a catalytic domain of a nuclease. In embodiments, a is selected from GHGG (SEQ ID NO: 31), HGSG (SEQ ID NO: 32), HGGG (SEQ ID NO: 33), GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60). In some embodiments, a gene-editing protein comprises a C-terminal GTHG (SEQ ID NO: 21) produces more efficient editing at the target locus than TALENs at 33° C. GTHG (SEQ ID NO: 21). In various embodiments, a gene-editing protein comprises a C-terminal GTHG (SEQ ID NO: 21) produces more efficient editing at the target locus than TALENs at 37° C.

Certain embodiments are directed to a nucleic acid molecule encoding a non-naturally occurring fusion protein comprising a first region that recognizes a predetermined nucleotide sequence and a second region with endonuclease activity, wherein the first region contains an artificial TAL effector repeat domain comprising one or more repeat units about 36 amino acids in length which differ from each other by no more than seven amino acids, and wherein the repeat domain is engineered for recognition of the predetermined nucleotide sequence. In one embodiment, the first region contains the amino acid sequence: LTPXQVVAIAS (SEQ ID NO: 29) where X can be either E or Q. In another embodiment, the amino acid sequence LTPXQVVAIAS (SEQ ID NO: 29) of the encoded non-naturally occurring fusion protein is immediately followed by an amino acid sequence selected from: HD, NG, NS, NI, NN, and N. In a further embodiment, the fusion protein comprises restriction endonuclease activity.

In embodiments, the gene-editing protein comprises (i) a DNA-binding domain comprising a plurality of repeat sequences and at least one of the repeat sequences comprises the amino acid sequence: LTPvQVVAIAwxyzHG, (SEQ ID NO: 30) wherein “v” is D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In another embodiment, the repeat sequence comprises: LTPvQVVAIAwxyzHG, (SEQ ID NO: 30) wherein “v” is D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwxyzHG, (SEQ ID NO: 30) wherein “v” is D or E, “w” is S or N, “x” is any amino acid other than N, H and I, “y” is any amino acid or no amino acid, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAIwyzHG (SEQ ID NO: 55), wherein “v” is D or E, “w” is S or N, “y” is any amino acid other than G, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwIAzHG (SEQ ID NO: 66), wherein “v” is D or E, “w” is S or N, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwxyzHG (SEQ ID NO: 30), wherein “v” is D or E, “w” is S or N, “x” is S, T or Q, “y” is any amino acid or no amino acid, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwxyzHG (SEQ ID NO: 30), wherein “v” is D or E, “w” is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), or GKQALETVQRLLPVLCQAHG (SEQ ID NO: 28). In yet another embodiment, the repeat sequence comprises: LTPvQVVAIAwx (SEQ ID NO: 67), wherein “v” is D or E, “w” is S or N, and “x” is S, T or Q. In yet another embodiment, the repeat sequence comprises: LTPvQVVAIAwxyz (SEQ ID NO: 16), wherein “v” is D or E, “w” is S or N, “x” is S, T or Q, and “y” is selected from: D, A, I, N, H, K, S, and G. In yet another embodiment, the repeat sequence comprises: LTPvQVVAIAwxyzGHGG (SEQ ID NO: 17), wherein “v” is Q, D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, “w” is S or N, “x” is any amino acid other than N, H and I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwyzGHGG (SEQ ID NO: 70), wherein “v” is Q, D or E, “w” is S or N, “y” is any amino acid other than G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwIAzGHGG, (SEQ ID NO: 71) wherein “v” is Q, D or E, “w” is S or N, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, “w” is S or N, “x” is S, T or Q, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwxyzGHGG (SEQ ID NO: 17), wherein “v” is Q, D or E, V is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In yet another embodiment, the repeat sequence comprises: LTPvQVVAIAwx (SEQ ID NO: 67), wherein “v” is Q, D or E, “w” is S or N, and “x” is S, T or Q. In yet another embodiment, the repeat sequence comprises: LTPvQVVAIAwxy (SEQ ID NO: 72), wherein “v” is Q, D or E, “w” is S or N, “x” is S, T or Q, and “y” is selected from: D, A, I, N, H, K, S, and G.

In some embodiments, the first gene editing protein and/or the second gene editing protein comprises a plurality of repeat sequences and at least one of the repeat sequences comprises the amino acid sequence: LTPvQVVAIAwxyz (SEQ ID NO: 16), wherein: (a) v is Q, D or E, (b) w is S or N, (c) x is I, H, or N, (d) y is D, A, I, N, H, K, S, G or null, and (e) z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20).

In some embodiments, wherein the first gene editing protein and/or the second gene editing protein comprises a plurality of repeat sequences and at least one of the repeat sequences comprises the amino acid sequence: LTPvQVVAIAwxyzα (SEQ ID NO: 22), wherein: (a) v is Q, D or E, (b) w is S or N, (c) x is I, H, or N, (d) y is D, A, I, N, H, K, S, G or null, (e) z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), and (f) α is any four consecutive amino acids.

In some embodiments, α comprises at least one glycine (G) residue. In some embodiments, a comprises at least one histidine (H) residue. In some embodiments, α comprises at least one histidine (H) residue at any one of positions 33, 34, or 35. In some embodiments, α comprises at least one aspartic acid (D) residue. In some embodiments, α comprises at least one, or two, or three of a glycine (G) residue, a histidine (H) residue, and an aspartic acid (D) residue. In some embodiments, a comprises one or more hydrophilic residues, optionally selected from: (a) a polar and positively charged hydrophilic amino acid, optionally selected from arginine (R) and lysine (K); (b) a polar and neutral of charge hydrophilic amino acid, optionally selected from asparagine (N), glutamine (Q), seine (S), threonine (T), proline (P), and cysteine (C); (c) a polar and negatively charged hydrophilic amino acid, optionally selected from aspartate (D) and glutamate (E), and (d) an aromatic, polar and positively charged hydrophilic amino acid, optionally selected from histidine (H). In some embodiments, α comprises one or more hydrophobic residues, optionally selected from: (a) a hydrophobic, aliphatic amino acid, optionally selected from glycine (G), alanine (A), leucine (L), isoleucine (I), methionine (M), and valine (V), and (b) a hydrophobic, aromatic amino acid, optionally selected from phenylalanine (F), tryptophan (W), and tyrosine (Y).

In some embodiments, a is selected from GHGG (SEQ ID NO: 31), HGSG (SEQ ID NO: 32), HGGG (SEQ ID NO: 33), GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60).

In some embodiments, the first gene editing protein and/or the second gene editing protein comprises: (a) a plurality of repeat sequences and at least one of the repeat sequences comprises the amino acid sequence: LTPvQVVAIAwxyzGHGG (SEQ ID NO: 17), wherein: “v” is Q, D or E, “w” is S or N, “x” is H, N, or I, “y” is D, A, I, N, G, H, K, S, G or null, and “z” is GGKQALETVQRLLPVLCQD (SEQ ID NO: 19) or GGKQALETVQRLLPVLCQA (SEQ ID NO: 20); and (b) a nuclease domain comprising a catalytic domain of a nuclease.

In some embodiments, first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is any amino acid other than N, H and I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In some embodiments, the first gene editing protein and/or the second gene editing protein comprises a plurality of repeat sequences and at least one of the repeat sequences comprises the amino acid sequence: LTPvQVVAIAwIyzGHGG (SEQ ID NO: 88), wherein “v” is Q, D or E, “w” is S or N, “y” is any amino acid other than G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In some embodiments, the first gene editing protein and/or the second gene editing protein comprises a plurality of repeat sequences and at least one of the repeat sequences comprises the amino acid sequence: LTPvQVVAIAwIAzGHGG (SEQ ID NO: 71), wherein “v” is Q, D or E, “w” is S or N, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68). In some embodiments, the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).

In some embodiments, the first gene editing protein and/or the second gene editing protein comprises a plurality of repeat sequences and at least one of the repeat sequences comprises the amino acid sequence: LTPvQVVAIAwx (SEQ ID NO: 67), wherein “v” is Q, D or E, “w” is S or N, and “x” is S, T or Q. In some embodiments, the first gene editing protein and/or the second gene editing protein comprises a plurality of repeat sequences and at least one of the repeat sequences comprises the amino acid sequence: LTPvQVVAIAwxy (SEQ ID NO: 72), wherein “v” is Q, D or E, “w” is S or N, “x” is S, T or Q, and “y” is selected from: D, A, I, N, H, K, S, and G.

The above-mentioned gene-editing proteins comprise a repeat variable di-residue (RVD) at residue 12 or 13, e.g., at “x” and “y” in the various above-mentioned repeat sequences, e.g., LTPvQVVAIAwxyzα (SEQ ID NO: 22), which targets the DNA-binding domain to a target DNA molecule. In embodiments, the RVD recognizes one base pair in the nucleic acid molecule. In embodiments, the RVD recognizes a C residue in the nucleic acid molecule and is selected from HD, N(null), HA, ND, and HI. In embodiments, the RVD recognizes a G residue in the nucleic acid molecule and is selected from NN, NH, NK, HN, and NA. In embodiments, the RVD recognizes an A residue in the nucleic acid molecule and is selected from Nl and NS. In embodiments, the RVD recognizes a T residue in the nucleic acid molecule and is selected from NG, HG, H(null), and IG.

In some embodiments, the RVD recognizing a C residue in the nucleic acid molecule is HD. In some embodiments, the RVD recognizing a C residue in the nucleic acid molecule is N(null). In some embodiments, the RVD recognizing a C residue in the nucleic acid molecule is HA. In some embodiments, the RVD recognizing a C residue in the nucleic acid molecule is ND. In some embodiments, the RVD recognizing a C residue in the nucleic acid molecule is HI. In some embodiments, the RVD recognizing a G residue in the nucleic acid molecule is NN. In some embodiments, the RVD recognizing a G residue in the nucleic acid molecule is NH. In some embodiments, the RVD recognizing a G residue in the nucleic acid molecule is NK. In some embodiments, the RVD recognizing a G residue in the nucleic acid molecule is HN. In some embodiments, the RVD recognizing a G residue in the nucleic acid molecule is NA. In some embodiments, the RVD recognizing an A residue in the nucleic acid molecule is Nl. In some embodiments, the RVD recognizing an A residue in the nucleic acid molecule is NS. In some embodiments, the RVD recognizing a T residue in the nucleic acid molecule is NG. In some embodiments, the RVD recognizing a T residue in the nucleic acid molecule is HG. In some embodiments, the RVD recognizing a T residue in the nucleic acid molecule is H(null). In some embodiments, the RVD recognizing a T residue in the nucleic acid molecule is IG.

In some embodiments, alternative DNA binding domains are employed.

For example, the alternative DNA binding domains described herein are, in embodiments, paired with the novel engineered nuclease domains described herein.

For example, the alternative DNA binding domains described herein are, in embodiments, used in the conditional activity: temperature dependence methods described herein.

For example, the alternative DNA binding domains described herein are, in embodiments, used in the conditional activity: methylation status methods described herein.

In embodiments, the engineered gene-editing proteins do not require a thymine (T) in the zero position of the target site (“To”).

In embodiments, the engineered gene-editing proteins that comprise DNA-binding domains comprise alterations in the in the N-terminal region to remove the T₀ requirement.

In embodiments, there is provided a method of gene-editing a cell with one or more of the present gene-editing proteins, optionally with also using a linear DNA repair template, optionally also using conditional activity methods described herein, where the target site lacks a thymine (T) in the zero position.

Wild type N-terminal region is characterized by the sequence: Asp225-IVGVGKQWSGARAL-Glu240 (DIVGVGKQWSGARALE; SEQ ID NO: 73). In embodiments, there is provided the engineered N-terminal region of Asp225-IVGVGKQKRGARAL-Glu240 (underling showing the change WS->KR) (DIVGVGKQKRGARALE; SEQ ID NO: 74).

In embodiments, there is provided an engineered N-terminal region in which KQWS is replaced with one or more amino acids, e.g., about 2-10 amino acids, or about 4-10 amino acids, or about 6-10 amino acids, or about 8-10 amino acids, or about 4 amino acids, or about 6 amino acids, or about 8 amino acids, or about 10 amino acids.

In embodiments, there is provided the engineered N-terminal region of Asp225-IVGVGGSKRGAGSGARAL-Glu244 (underling showing the change KQWS->GSKRGAGS) (DIVGVGGSKRGAGSGARALE; SEQ ID NO: 75).

In some cases, a cell is contacted with a demethylating agent during the process of gene-editing. In embodiments, the demethylating agent is selected from 5-azacitidine and 5-aza-2′-deoxycitidine (decitabine).

In some embodiments, the first gene editing protein and/or the second gene editing protein comprises: (a) the DNA-binding domain comprising a plurality of repeat sequences and at least one of the repeat sequences comprises a repeat variable di-residue (RVD) at residue 12 or 13; and (b) the nuclease domain comprising a catalytic domain, the catalytic domain comprising a hybrid of the catalytic domains of FokI and StsI, comprising the α1, α2, α3, α4, α5, α6, β1, β2, β3, β4, β5, and β6 domains of FokI with at least one of the domains of FokI being substituted in whole or in part with the α1, α2, α3, α4, α5, α6, β1, β2, β3, β4, β5, and β6 domains of StsI and optionally comprising at least one mutation, e.g., in the catalytic site of the nuclease thereby preventing the mutant nuclease domain from creating a break in a DNA site. In embodiments, the nuclease domain is capable of binding to the DNA and forming a dimer with another nuclease domain. In embodiments, the nuclease domain comprising a mutation is incapable of incapable of creating a single-stranded break in a DNA site.

In some embodiments, certain fragments of an endonuclease cleavage domain are used, including fragments that are truncated at the N-terminus, fragments that are truncated at the C-terminus, fragments that have internal deletions, and fragments that combine N-terminus, C-terminus, and/or internal deletions, which maintain part or all of the catalytic activity of the full endonuclease cleavage domain. Determining whether a fragment can maintain part, or all of the catalytic activity of the full domain can be accomplished by, for example, synthesizing a gene-editing protein that contains the fragment according to the methods of the present invention, inducing cells to express the gene-editing protein according to the methods of the present invention, and measuring the efficiency of gene editing. In some embodiments, a measurement of gene-editing efficiency is used to ascertain whether any specific fragment maintains part or all of the catalytic activity of the full endonuclease cleavage domain. Certain embodiments are therefore directed to a biologically active fragment of an endonuclease cleavage domain. In one embodiment, the endonuclease cleavage domain is selected from: FokI, StsI, StsI-HA, StsI-HA2, StsI-UHA, StsI-UHA2, StsI-HF, and StsI-UHF or a natural or engineered variant or biologically active fragment thereof, or a hybrid or chimera thereof.

In some embodiments, the nuclease domain incapable of creating a single-stranded break in a DNA site comprises a FokI domain. In some embodiments, the FokI domain bears at least one mutation. In some embodiments, the mutation is selected from the group consisting of D67A, D67N, and D84A, each of which are numbered in reference to the sequence of SEQ ID NO: 80. In some embodiments, the mutation is a D67A mutation and the FokI domain comprises a sequence with at least 60%, at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 81. In some embodiments, the FokI domain is encoded by a nucleic acid comprising a sequence with at least 60%, at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 85.

In some embodiments, the mutation is a D67N mutation and the FokI domain comprises a sequence with at least 60%, at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 82. In some embodiments, the FokI domain is encoded by a nucleic acid comprising a sequence with at least 60%, at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 86. In some embodiments, the mutation is a D67A mutation and the FokI domain comprises a sequence with at least 60%, at least 70%, at least 80%, at least 85%, at least 90%, at least 91%, at least 92%, at least 93%, at least 94%, at least 95%, at least 96%, at least 97%, at least 98%, at least 99% or 100% sequence identity to SEQ ID NO: 83. In some embodiments, the FokI domain is encoded by a nucleic acid comprising a sequence with at least 80% sequence identity to SEQ ID NO: 87.

In embodiments, the gene-editing protein comprises a linker. In another embodiment, the linker connects a DNA-binding domain to a nuclease domain. In a further embodiment, the linker is between about 1 and about 10 amino acids long. In some embodiments, the linker is about 1, about 2, or about 3, or about 4, or about 5, or about 6, or about 7, or about 8, or about 9, or about 10 amino acids long. In one embodiment, the gene-editing protein is capable of generating a nick or a double-strand break in a target DNA molecule.

In embodiments, the gene-editing protein is any of those described in International Patent Publication No. US20150267189A1 or US20230193231A1, hereby incorporated by reference in their entireties.

In various embodiments, the cell is transfected (e.g., contacted) with a synthetic nucleic acid encoding the gene-editing protein at about 30° C. to about 35° C., e.g., without limitation about 33° C. In embodiments, the contacting occurs at about 30° C. In some embodiments, the contacting occurs at about 31° C. In some embodiments, the contacting occurs at about 32° C. In some embodiments, the contacting occurs at about 33° C. In some embodiments, the contacting occurs at about 34° C. In some embodiments, the contacting occurs at about 35° C. In embodiments, the gene-editing protein is functionally temperature-switchable. In embodiments, the method further comprises the step of (c) culturing the contacted cell at about 30° C. to about 35° C. In embodiments, the method further comprises the step of (c) culturing the contacted cell at about 30° C. In embodiments, the method further comprises the step of (c) culturing the contacted cell at about 31° C. In embodiments, the method further comprises the step of (c) culturing the contacted cell at about 32° C. In embodiments, the method further comprises the step of (c) culturing the contacted cell at about 33° C. In embodiments, the method further comprises the step of (c) culturing the contacted cell at about 34° C. In embodiments, the method further comprises the step of (c) culturing the contacted cell at about 35° C.

Further description of temperature-sensitive gene-editing is found in US20230193231A1. The entire contents of which are incorporated by reference in their entirety.

In embodiments, the synthetic nucleic acid encoding the gene-editing protein is transfected along with a repair template. In some cases, the repair template is a double stranded synthetic oligodeoxynucleotide (dsODNs). In embodiments, the dsODNs comprises a repair template and comprises the TTAGGG motif. In some cases, the dsODN comprises a repair template and lacks the TTAGGG motif and a separate dsODNs comprising the TTAGGG motif is transfected into the cell.

In embodiments, the RNA is mRNA. In embodiments, the RNA is modified mRNA. In embodiments, the modified mRNA comprises one or more non-canonical nucleotides. In some embodiments, non-canonical nucleotides are incorporated into RNA to increase the efficiency with which the RNA can be translated into protein, and can decrease the toxicity of the RNA. In embodiments, the RNA molecule comprises one or more non-canonical nucleotides. In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 20 nucleotides to about 250 nucleotides.

In some embodiments, the synthetic RNA comprises about 200 nucleotides to about 5000 nucleotides. In some embodiments, the synthetic RNA comprises from about 500 to about 2000 nucleotides, or about 500 to about 1500 nucleotides, or about 500 to about 1000 nucleotides.

In various cases, transfection of a cell with synthetic nucleic acids for gene-editing the cell may be facilitated by use of the ToRNAdo™ Nucleic-Acid Delivery System. This system relates to new lipids that find use, inter alia, in improved delivery of biological payloads, e.g., nucleic acids, to cells. The system relates to use of a compound of Formula (IV)

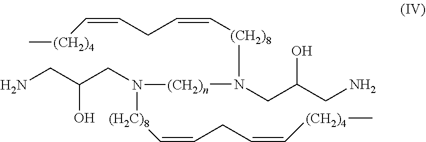

where n is 1, 2, 3, 4, 5, 6, 7, 8, 9, 10, 11, 12, 13, 14, or 15. Further description of ToRNAdo™ Nucleic-Acid Delivery System is found in one or both of U.S. Pat. No. 10,501,404 and US20210009505A1. The entire contents of which are incorporated by reference in their entirety.

In any of the herein-disclosed aspects or embodiments, a synthetic RNA molecule encoding the gene-editing protein may be in the form of a circular RNA (circRNA). The circRNA are manufactured by methods do not require a linear oligonucleotide (splint) to pre-orient the two reacting ends of a linear RNA to assist in ligation to yield a circRNA, the circRNA are manufactured by methods that do not require ribozymes to yield a circRNA, and/or the circRNA are manufactured by methods that do not require HPLC-based purification, e.g., post-ligation. A nucleic acid that can be manufactured into a circRNA has the structure: 5′-X-Y-A-IRES-B-CDS-C-Y′-Z 3′. Here, Y and Y′ each independently comprise one or more nucleotides and Y and Y′ are substantially complementary; X and Z each independently comprise one or more nucleotides and X and Z are not substantially complementary; IRES comprises an internal ribosome entry site; CDS comprises a coding sequence; and A, B, and C are each independently a spacer comprising one or more nucleotides or null. The CDS of a circRNA encodes the gene-editing protein(s). Additional details regarding circRNAs useful in the present disclosure are described in PCT/US2022/026564, the contents of which are incorporated herein by reference in its entirety.

In some embodiments, the synthetic RNA comprises a 5′ cap structure. In some embodiments, the synthetic RNA comprises a Kozak consensus sequence. In some embodiments, the synthetic RNA comprises a 5′-UTR which comprises a sequence that increases RNA stability in vivo, and the 5′-UTR optionally comprises an alpha-globin or beta-globin 5′-UTR. In some embodiments, the synthetic RNA comprises a 3′-UTR which comprises a sequence that increases RNA stability in vivo, and the 3′-UTR optionally comprises an alpha-globin or beta-globin 3′-UTR. In some embodiments, the synthetic RNA comprises a 5′-UTR which comprises a microRNA binding site that modulates RNA stability in a cell type-specific manner. In some embodiments, the synthetic RNA comprises a 3′-UTR which comprises a microRNA binding site that modulates RNA stability in a cell type-specific manner. In some embodiments, the synthetic RNA comprises a 3′ poly(A) tail. In some embodiments, the synthetic RNA comprises a 3′ poly(A) tail which comprises from about 20 nucleotides to about 250 nucleotides.

Certain embodiments are directed to a nucleic acid comprising a 5′-cap structure selected from Cap 0, Cap 1, Cap 2, and Cap 3 or a derivative thereof. In one embodiment, the nucleic acid comprises one or more UTRs. In another embodiment, the one or more UTRs increase the stability of the nucleic acid. In a further embodiment, the one or more UTRs comprise an alpha-globin or beta-globin 5′-UTR. In a still further embodiment, the one or more UTRs comprise an alpha-globin or beta-globin 3′-UTR. In a still further embodiment, the RNA molecule comprises an alpha-globin or beta-globin 5′-UTR and an alpha-globin or beta-globin 3′-UTR. In one embodiment, the 5′-UTR comprises a Kozak sequence that is substantially similar to the Kozak consensus sequence. In another embodiment, the nucleic acid comprises a 3′-poly(A) tail. In a further embodiment, the 3′-poly(A) tail is between about 20nt and about 250nt or between about 120nt and about 150nt long. In a further embodiment, the 3′-poly(A) tail is about 20nt, or about 30nt, or about 40nt, or about 50nt, or about 60nt, or about 70nt, or about 80nt, or about 90nt, or about 100nt, or about 110nt, or about 120nt, or about 130nt, or about 140nt, or about 150nt, or about 160nt, or about 170nt, or about 180nt, or about 190nt, or about 200nt, or about 210nt, or about 220nt, or about 230nt, or about 240nt, or about 250nt long.

In some embodiments, the RNA comprises a tail composed of a plurality of adenines with one or more guanines.

In embodiments, the RNA comprises (a) a sequence encoding a protein, and (b) a tail region comprising deoxyadenosine nucleotides and one or more other nucleotides.

In embodiments, the one or more other nucleotides comprises deoxyguanosine residues. In embodiments, the tail region comprises about 1%, about 2%, about 5%, about 10%, about 15%, about 20%, about 25%, about 30%, about 35%, about 40%, about 45%, or about 50% deoxyguanosine residues. In embodiments, the tail region comprises more than 50% deoxyguanosine residues.

In embodiments, the one or more other nucleotides comprises deoxycytidine residues. In embodiments, the tail region comprises about 1%, about 2%, about 5%, about 10%, about 15%, about 20%, about 25%, about 30%, about 35%, about 40%, about 45%, or about 50% deoxycytidine residues. In embodiments, the tail region comprises more than 50% deoxycytidine residues.

In embodiments, the one or more other nucleotides comprises deoxythymidine residues. In embodiments, the tail region comprises about 1%, about 2%, about 5%, about 10%, about 15%, about 20%, about 25%, about 30%, about 35%, about 40%, about 45%, or about 50% deoxythymidine residues. In embodiments, the tail region comprises more than 50% deoxythymidine residues.

In embodiments, the one or more other nucleotides comprise deoxyguanosine residues and deoxycytidine residues. In embodiments, the tail region comprises about 99%, about 98%, about 95%, about 90%, about 85%, about 80%, about 75%, about 70%, about 65%, about 60%, about 55%, or about 50% deoxyadenosine residues. In embodiments, the tail region comprises fewer than 50% deoxyadenosine residues.

In embodiments, the one or more other nucleotides comprises guanosine residues.

In embodiments, the tail region comprises about 1%, about 2%, about 5%, about 10%, about 15%, about 20%, about 25%, about 30%, about 35%, about 40%, about 45%, or about 50% guanosine residues. In embodiments, the tail region comprises more than 50% guanosine residues.

In embodiments, the one or more other nucleotides comprises cytidine residues. In embodiments, the tail region comprises about 1%, about 2%, about 5%, about 10%, about 15%, about 20%, about 25%, about 30%, about 35%, about 40%, about 45%, or about 50% cytidine residues. In embodiments, the tail region comprises more than 50% cytidine residues.

In embodiments, the one or more other nucleotides comprises uridine residues. In embodiments, the tail region comprises about 1%, about 2%, about 5%, about 10%, about 15%, about 20%, about 25%, about 30%, about 35%, about 40%, about 45%, or about 50% uridine residues. In embodiments, the tail region comprises more than 50% uridine residues.

In embodiments, the one or more other nucleotides comprise guanosine residues and cytidine residues. In embodiments, the tail region comprises about 99%, about 98%, about 95%, about 90%, about 85%, about 80%, about 75%, about 70%, about 65%, about 60%, about 55%, or about 50% adenosine residues.

In embodiments, the tail region comprises fewer than 50% adenosine residues.

In embodiments, the tail is (A)₁₅₀ (SEQ ID NO: 61). In embodiments, the tail is (A₃₉G)₃(A)₃₀ (SEQ ID NO: 62). In embodiments, the tail is (A₁₉G)₇(A)₁₀ (SEQ ID NO: 63). In embodiments, the tail is (A₉G)₁₅ (SEQ ID NO: 64).

In embodiments, the length of the tail region is between about 80 nucleotides and about 120 nucleotides, about 120 nucleotides and about 160 nucleotides, about 160 nucleotides and about 200 nucleotides, about 200 nucleotides and about 240 nucleotides, about 240 nucleotides and about 280 nucleotides, or about 280 nucleotides and about 320 nucleotides.

In embodiments, the length of the tail region is greater than 320 nucleotides.

In embodiments, the RNA comprises a 5′ cap structure. In embodiments, the RNA 5′-UTR comprises a Kozak consensus sequence. In embodiments, the RNA 5′-UTR comprises a sequence that increases RNA stability in vivo, and the 5′-UTR may comprise an alpha-globin or beta-globin 5′-UTR.

In embodiments, the RNA 3′-UTR comprises a sequence that increases RNA stability in vivo, and the 3′-UTR may comprise an alpha-globin or beta-globin 3′-UTR. In embodiments, the RNA comprises a 3′ poly(A) tail. In embodiments, the RNA 3′ poly(A) tail is from about 20 nucleotides to about 250 nucleotides in length.

In embodiments, the RNA is from about 200 nucleotides to about 5000 nucleotides in length.

In embodiments, the RNA is prepared by in vitro transcription. In embodiments, the RNA is synthetic.

In some embodiments, the synthetic RNA comprises about 200 nucleotides to about 5000 nucleotides. In some embodiments, the synthetic RNA comprises from about 500 to about 2000 nucleotides, or about 500 to about 1500 nucleotides, or about 500 to about 1000 nucleotides.

Further description of gene-editing is found in one or more of US20140356906A1, US20150267189A1, US20180021412A1, US20180256748A1, US20190008985A1, US20210024907A1, US20210009505A1, US20230193231A1, or US20230193207A1. The entire contents of which are incorporated by reference in their entirety.

### RNA Modifications

In embodiments, the present disclosure relates to RNA-based modifications, e.g., reprogramming and/or gene-editing. In some embodiments, an RNA molecule encodes a gene-editing protein. In some embodiments, a RNA molecule encodes a reprogramming factor.

In embodiments, the RNA is mRNA. In embodiments, the RNA is modified mRNA. In embodiments, the modified mRNA comprises one or more non-canonical nucleotides.

In some embodiments, non-canonical nucleotides are incorporated into RNA to increase the efficiency with which the RNA can be translated into protein, and can decrease the toxicity of the RNA. In embodiments, the RNA molecule comprises one or more non-canonical nucleotides.

In some embodiments, the synthetic RNA molecule contains one or more non-canonical nucleotides that include one or more substitutions at the 2C and/or 4C and/or 5C positions in the case of a pyrimidine or the 6C and/or 7N and/or 8C positions in the case of a purine can be less toxic than synthetic RNA molecules containing only canonical nucleotides, due in part to the ability of substitutions at these positions to interfere with recognition of synthetic RNA molecules by proteins that detect exogenous nucleic acids, and furthermore, that substitutions at these positions can have minimal impact on the efficiency with which the synthetic RNA molecules can be translated into protein, due in part to the lack of interference of substitutions at these positions with base-pairing and base-stacking interactions.

In embodiments, the synthetic RNA molecule is mRNA comprising one or more non-canonical nucleotides selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In some embodiments, the one or more non-canonical nucleotides are selected from 5-hydroxycytidine, 5-methylcytidine, 5-hydroxymethylcytidine, 5-carboxycytidine, 5-formylcytidine, 5-methoxycytidine, 5-hydroxyuridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine. In some embodiments, at least 50%, or at least 55%, or at least 60%, or at least 65%, or at least 70%, or at least 75%, or at least 80%, or at least 85%, or at least 90%, or at least 95%, or 100% of the non-canonical nucleotides are one or more of 5-hydroxycytidine, 5-methylcytidine, 5-hydroxymethylcytidine, 5-carboxycytidine, 5-formylcytidine, 5-methoxycytidine, 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 50%, or at least about 55%, or at least 60%, or at least 65%, or at least 70%, or at least 75%, or at least 80%, or at least 85%, or at least 90%, or at least 95%, or 100% of cytidine residues are non-canonical nucleotides selected from 5-hydroxycytidine, 5-methylcytidine, 5-hydroxymethylcytidine, 5-carboxycytidine, 5-formylcytidine, 5-methoxycytidine.

In some embodiments, at least about 20%, or about 30%, or about 40%, or about 50%, or at least about 55%, or at least 60%, or at least 65%, or at least 70%, or at least 75%, or at least 80%, or at least 85%, or at least 90%, or at least 95%, or 100% of uridine residues are non-canonical nucleotides selected from 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 10% (e.g., 10%, or about 20%, or about 30%, or about 40%, or about 50%) of guanosine residues are non-canonical nucleotides, and the non-canonical nucleotide is optionally 7-deazaguanosine. In some embodiments, the RNA contains no more than about 50% 7-deazaguanosine in place of guanosine residues.

In some embodiments, the synthetic RNA molecule does not contain non-canonical nucleotides in place of adenosine residues.

Other non-canonical nucleotides that can be used in place of or in combination with 5-methyluridine include but are not limited to: pseudouridine and 5-methylpseudouridine (a.k.a. “1-methylpseudouridine”, a.k.a. “N1-methylpseudouridine”) or one or more derivatives thereof. Other non-canonical nucleotides that can be used in place of or in combination with 5-methylcytidine and/or 5-hydroxymethylcytidine include, but are not limited to: pseudoisocytidine, 5-methylpseudoisocytidine, 5-hydroxymethylcytidine, 5-formylcytidine, 5-carboxycytidine, N4-methylcytidine, N4-acetylcytidine or one or more derivatives thereof. In certain embodiments, for example, when performing only a single transfection or when the cells being transfected are not particularly sensitive to transfection-associated toxicity or innate-immune signaling, the fractions of non-canonical nucleotides can be reduced. Reducing the fraction of non-canonical nucleotides can be beneficial, in part, because reducing the fraction of non-canonical nucleotides can reduce the cost of the nucleic acid. In certain situations, for example, when minimal immunogenicity of the nucleic acid is desired, the fractions of non-canonical nucleotides can be increased.

Note that alternative naming schemes exist for certain non-canonical nucleotides. For example, in certain situations, 5-methylpseudouridine can be referred to as “3-methylpseudouridine” or “N3-methylpseudouridine” or “1-methylpseudouridine” or “Ni-methylpseudouridine”. Nucleotides that contain the prefix “amino” can refer to any nucleotide that contains a nitrogen atom bound to the atom at the stated position of the nucleotide, for example, 5-aminocytidine can refer to 5-aminocytidine, 5-methylaminocytidine, and 5-nitrocytidine. Similarly, nucleotides that contain the prefix “methyl” can refer to any nucleotide that contains a carbon atom bound to the atom at the stated position of the nucleotide, for example, 5-methylcytidine can refer to 5-methylcytidine, 5-ethylcytidine, and 5-hydroxymethylcytidine, nucleotides that contain the prefix “thio” can refer to any nucleotide that contains a sulfur atom bound to the atom at the given position of the nucleotide, and nucleotides that contain the prefix “hydroxy” can refer to any nucleotide that contains an oxygen atom bound to the atom at the given position of the nucleotide, for example, 5-hydroxyuridine can refer to 5-hydroxyuridine and uridine with a methyl group bound to an oxygen atom, wherein the oxygen atom is bound to the atom at the 5C position of the uridine.

In some embodiments, non-canonical nucleotides are incorporated into RNA to increase the efficiency with which the RNA can be translated into protein and can decrease the toxicity of the RNA. In embodiments, the RNA molecule comprises one or more non-canonical nucleotides. In some embodiments, the nucleic acid comprises one or more non-canonical nucleotide members of the 5 methylcytidine de-methylation pathway. In some embodiments, the nucleic acid comprises at least one of 5 methylcytidine, 5 hydroxymethylcytidine, 5 formylcytidine, and 5 carboxycytidine or a derivative thereof. In some embodiments, the nucleic acid comprises at least one of pseudouridine, 5 methylpseudouridine, 5 methyluridine, 5 methylcytidine, 5 hydroxymethylcytidine, N4-methylcytidine, N4-acetylcytidine, and 7-deazaguanosine or a derivative thereof.

Certain non-canonical nucleotides can be incorporated more efficiently than other non-canonical nucleotides into RNA molecules by RNA polymerases that are commonly used for in vitro transcription, due in part to the tendency of these certain non-canonical nucleotides to participate in standard base-pairing interactions and base-stacking interactions, and to interact with the RNA polymerase in a manner similar to that in which the corresponding canonical nucleotide interacts with the RNA polymerase. As a result, certain nucleotide mixtures containing one or more non-canonical nucleotides can be beneficial in part because in vitro-transcription reactions containing these nucleotide mixtures can yield a large quantity of RNA. Certain embodiments are therefore directed to a nucleotide mixture containing one or more nucleotides that includes one or more substitutions at the 2C and/or 4C and/or 5C positions in the case of a pyrimidine or the 6C and/or 7N and/or 8C positions in the case of a purine. Nucleotide mixtures include, but are not limited to (numbers preceding each nucleotide indicate an exemplary fraction of the non-canonical nucleotide triphosphate in an in vitro-transcription reaction, for example, 0.2 pseudoisocytidine refers to a reaction containing adenosine-5′-triphosphate, guanosine-5′-triphosphate, uridine-5′-triphosphate, cytidine-5′-triphosphate, and pseudoisocytidine-5′-triphosphate, wherein pseudoisocytidine-5′-triphosphate is present in the reaction at an amount approximately equal to 0.2 times the total amount of pseudoisocytidine-5′-triphosphate+cytidine-5′-triphosphate that is present in the reaction, with amounts measured either on a molar or mass basis, and wherein more than one number preceding a nucleoside indicates a range of exemplary fractions): 1.0 pseudouridine, 0.1-0.8 2-thiouridine, 0.1-0.8 5-methyluridine, 0.2-1.0 5-hydroxyuridine, 0.2-1.0 5-methoxyuridine, 0.1-1.0 5-aminouridine, 0.1-1.0 4-thiouridine, 0.1-1.0 2-thiopseudouridine, 0.1-1.0 4-thiopseudouridine, 0.1-1.0 5-hydroxypseudouridine, 0.2-1 5-methylpseudouridine, 0.2-1.0 5-methoxypseudouridine, 0.1-1.0 5-aminopseudouridine, 0.2-1.0 2-thiocytidine, 0.1-0.8 pseudoisocytidine, 0.2-1.0 5-methylcytidine, 0.2-1.0 5-hydroxycytidine, 0.2-1.0 5-hydroxymethylcytidine, 0.2-1.0 5-methoxycytidine, 0.1-1.0 5-aminocytidine, 0.2-1.0 N4-methylcytidine, 0.2-1.0 5-methylpseudoisocytidine, 0.2-1.0 5-hydroxypseudoisocytidine, 0.2-1.0 5-aminopseudoisocytidine, 0.2-1.0 N4-methylpseudoisocytidine, 0.2-1.0 2-thiopseudoisocytidine, 0.2-1.0 7-deazaguanosine, 0.2-1.0 6-thioguanosine, 0.2-1.0 6-thio-7-deazaguanosine, 0.2-1.0 8-azaguanosine, 0.2-1.0 7-deaza-8-azaguanosine, 0.2-1.0 6-thio-8-azaguanosine, 0.1-0.5 7-deazaadenosine, and 0.1-0.5 N6-methyladenosine.

In some embodiments, the RNA comprising one or more non-canonical nucleotides composition or synthetic polynucleotide composition (e.g., which may be prepared by in vitro transcription) contains substantially or entirely the canonical nucleotide at positions having adenine or “A” in the genetic code. The term “substantially” in this context refers to at least 90%. In these embodiments, the RNA composition or synthetic polynucleotide composition may further contain (e.g., consist of) 7-deazaguanosine at positions with “G” in the genetic code as well as the corresponding canonical nucleotide “G”, and the canonical and non-canonical nucleotide at positions with G may be in the range of 5:1 to 1:5, or in some embodiments in the range of 2:1 to 1:2. In these embodiments, the RNA composition or synthetic polynucleotide composition may further contain (e.g., consist of) one or more (e.g., two, three or four) of 5-hydroxycytidine, 5-methylcytidine, 5-hydroxymethylcytidine, 5-carboxycytidine, 5-formylcytidine, 5-methoxycytidine at positions with “C” in the genetic code as well as the canonical nucleotide “C”, and the canonical and non-canonical nucleotide at positions with C may be in the range of 5:1 to 1:5, or in some embodiments in the range of 2:1 to 1:2. In some embodiments, the level of non-canonical nucleotide at positions of “C” are as described in the preceding paragraph. In these embodiments, the RNA composition or synthetic polynucleotide composition may further contain (e.g., consist of) one or more (e.g., two, three, or four) of 5-hydroxyuridine, 5-methyluridine, 5-hydroxymethyluridine, 5-carboxyuridine, 5-formyluridine, 5-methoxyuridine, pseudouridine, 5-hydroxypseudouridine, 5-methylpseudouridine, 5-hydroxymethylpseudouridine, 5-carboxypseudouridine, 5-formylpseudouridine, and 5-methoxypseudouridine at positions with “U” in the genetic code as well as the canonical nucleotide “U”, and the canonical and non-canonical nucleotide at positions with “U” may be in the range of 5:1 to 1:5, or in some embodiments in the range of 2:1 to 1:2. In some embodiments, the level of non-canonical nucleotide at positions of “U” are as described in the preceding paragraph.

In embodiments, combining certain non-canonical nucleotides can be beneficial in part because the contribution of non-canonical nucleotides to lowering the toxicity of RNA molecules can be additive. Certain embodiments are therefore directed to a nucleotide mixture, wherein the nucleotide mixture contains more than one of the non-canonical nucleotides listed above, for example, the nucleotide mixture contains both pseudoisocytidine and 7-deazaguanosine or the nucleotide mixture contains both N4-methylcytidine and 7-deazaguanosine, etc. In one embodiment, the nucleotide mixture contains more than one of the non-canonical nucleotides listed above, and each of the non-canonical nucleotides is present in the mixture at the fraction listed above, for example, the nucleotide mixture contains 0.1-0.8 pseudoisocytidine and 0.2-1.0 7-deazaguanosine or the nucleotide mixture contains 0.2-1.0 N4-methylcytidine and 0.2-1.0 7-deazaguanosine, etc.

In certain situations, for example, when it may not be necessary or desirable to maximize the yield of an in vitro-transcription reaction, nucleotide fractions other than those given above may be used. The exemplary fractions and ranges of fractions listed above relate to nucleotide-triphosphate solutions of typical purity (greater than 90% purity). Larger fractions of these and other nucleotides can be used by using nucleotide-triphosphate solutions of greater purity, for example, greater than about 95% purity or greater than about 98% purity or greater than about 99% purity or greater than about 99.5% purity, which can be achieved, for example, by purifying the nucleotide triphosphate solution using existing chemical-purification technologies such as high-pressure liquid chromatography (HPLC) or by other means. In one embodiment, nucleotides with multiple isomers are purined to enrich the desired isomer.

In some embodiments, the one or more non-canonical nucleotides avoids substantial cellular toxicity.

In some embodiments, the non-canonical nucleotides comprise one or more of 5-hydroxycytidine, 5-methylcytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine, optionally at an amount of at least 50%, or at least 60%, or at least 70%, or at least 80%, or at least 90%, or 100% of the non-canonical nucleotides.

In some embodiments, at least about 50% of cytidine residues are non-canonical nucleotides, and which are selected from 5-hydroxycytidine, 5-methylcytidine, 5-hydroxymethylcytidine, 5-carboxycytidine, 5-formylcytidine, and 5-methoxycytidine.

In some embodiments, at least about 75% or at least about 90% of cytidine residues are non-canonical nucleotides, and the non-canonical nucleotides are selected from 5-hydroxycytidine, 5-methylcytidine, 5-hydroxymethylcytidine, 5-carboxycytidine, 5-formylcytidine, and 5-methoxycytidine.

In some embodiments, at least about 20% of uridine, or at least about 40%, or at least about 50%, or at least about 75%, or at about least 90% of uridine residues are non-canonical nucleotides, and the non-canonical are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 40%, or at least about 50%, or at least about 75%, or at about least 90% of uridine residues are non-canonical nucleotides, and the non-canonical nucleotides are selected from pseudouridine, 5-hydroxyuridine, 5-methyluridine, 5-hydroxymethyluridine, 5-carboxyuridine, 5-formyluridine, 5-methoxyuridine, 5-hydroxypseudouridine, 5-methylpseudouridine, 5-hydroxymethylpseudouridine, 5-carboxypseudouridine, 5-formylpseudouridine, and 5-methoxypseudouridine.

In some embodiments, at least about 10% of guanine residues are non-canonical nucleotides, and the non-canonical nucleotide is optionally 7-deazaguanosine. In some embodiments, the synthetic RNA comprises no more than about 50% 7-deazaguanosine in place of guanosine residues. In some embodiments, the synthetic RNA does not comprise non-canonical nucleotides in place of adenosine residues.

### Methods of Treatment

An aspect of the present disclosure is a method for treating a disease. The method comprising administering to a subject in need a therapeutically-effective amount of genetically-engineered cells for treating a disease.

In various embodiments, the method further comprises administering to the subject in need a synthetic mRNA encoding a gene-editing protein (e.g., a temperature-sensitive gene-editing protein) and a single-stranded or double-stranded repair template which encodes a protein of interest. In some cases, the gene-editing protein creates a single-stranded break or a double-stranded break in the genomic DNA of a cell in the subject and the single-stranded or double-stranded repair template which encodes the protein of interest inserts into the break. In these embodiments, the cell in the subject expresses or over expresses the protein of interest.

When the synthetic mRNA and/or the repair template is administered to a subject, the synthetic mRNA and/or the repair is combined with a lipid system comprising a compound of Formula (IV).

In various embodiments, the present methods and compositions find use in methods of treating, preventing, or ameliorating a disease, disorder, and/or condition. For instance, in some embodiments, the described methods of in vivo delivery, including administration strategies, and formulations are used in a method of treatment. In some methods, the described methods reduce symptoms associated with a disease. In some embodiments, the methods eliminate the underlying cause of the disease. In some embodiments, the methods are used in the treatment of a disease requiring immunosuppression. In some embodiments, the methods reduce inflammation. In some embodiments, the methods reduce immune response.

In various embodiments, the present invention pertains to pharmaceutical compositions comprising the recombinantly engineered cells described herein and a pharmaceutically acceptable carrier or excipient. In some embodiments, the present invention pertains to pharmaceutical compositions comprising the iPSC-derived cells of the lymphoid lineage, including cytotoxic lymphocytes, iPSC-derived cells of the myeloid lineage, e.g., monocytes which can be differentiated into functional M1 and M2 macrophages having enhanced cytokine secretion and tumor cell-killing activity, and/or synthetic RNA molecules encoding the gene-editing protein or expression cassettes for expressing a protein of interest, e.g., a CAR or for expressing or overexpressing a cytokine.

In embodiments, the disclosed composition is suitable for use in the treatment of amyotrophic lateral sclerosis (ALS), spinal cord injury, degenerative disc disease, coronary artery disease, acute myocardial infarction, alcoholic liver cirrhosis, hepatitis C virus (HCV)-induced cirrhosis, multiple sclerosis (MS), osteoarthritis (OA), osteoarthritis of the knee, kidney allograft, critical limb ischemia, ischemic cardiomyopathy, Crohn's disease, idiopathic pulmonary fibrosis, anal fistula, spinal cord injury, systemic lupus erythematosus (SLE), acute respiratory distress syndrome (ARDS), acute graft-versus-host disease (aGvHD), preterm bronchopulmonary dysplasia (BPD), autismnonischemic heart failure, and/or Type 2 diabetes mellitus.

In embodiments, the present methods relate to therapeutic use in autoimmune diseases or disorders.

Examples of autoimmune diseases or disorders that may be treated or prevented by the present invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre syndrome, Hashimoto's thyroiditis, idiopathic pulmonary fibrosis, idiopathic thrombocytopenia purpura (ITP), irritable bowel disease (IBD), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, stiff-man syndrome, systemic lupus erythematosus, lupus erythematosus, takayasu arteritis, temporal arteristis, giant cell arteritis, ulcerative colitis, uveitis, vitiligo and Wegener's granulomatosis. Preferably autoimmune disorders that may be treated or prevented by the present compositions include rheumatoid arthritis, type 1 diabetes mellitus, multiple sclerosis, systemic lupus erythematosus, and atopy.

In embodiments, the present methods relate to therapeutic use in degenerative diseases or disorders.

A degenerative diseases or disorders is a disease in which the function or structure of the affected tissues or organs will progressively deteriorate over time. Examples of degenerative diseases that can be treated or prevented with the present invention include Amyotrophic Lateral Sclerosis (ALS), Alzheimer's disease, Parkinson's Disease, Multiple system atrophy, Niemann Pick disease, Atherosclerosis, Progressive supranuclear palsy, Tay-Sachs Disease, Diabetes, Heart Disease, Keratoconus, Inflammatory Bowel Disease (IBD), Prostatitis, Osteoarthritis, Osteoporosis, Rheumatoid Arthritis, Huntington's Disease, Chronic traumatic encephalopathy, Epilepsy, Dementia, Renal failure, Multiple sclerosis, Malaria with CNS degeneration, Neuro-AIDS, Lysosomal storage diseases, Encephalatis of viral, bacterial or autoimmune origin.

In embodiments, the present methods relate to therapeutic use in a lung diseases or disorders.

In embodiments, the lung disease or disorder is a lung disease or disorder that would benefit therapeutically from suppression of immune responses in the lung. In some embodiments, inflammation is associated with the lung disease or disorder.

In some embodiments, the lung disease or disorder is selected from Asbestosis, Asthma, Bronchiectasis, Bronchitis, Chronic Cough, Chronic Obstructive Pulmonary Disease (COPD), Common Cold, Croup, Cystic Fibrosis, Hantavirus, Idiopathic Pulmonary Fibrosis, Influenza, Lung Cancer, Pandemic Flu, Pertussis, Pleurisy, Pneumonia, Pulmonary Embolism, Pulmonary Hypertension, Respiratory Syncytial Virus (RSV), Sarcoidosis, Sleep Apnea, Spirometry, Sudden Infant Death Syndrome (SIDS), and Tuberculosis.

In some embodiments, the lung disease or disorder is chronic obstructive pulmonary disease (COPD), reactive airway disease such as asthma, bronchiolitis, acute lung injury, lung allograft rejection (acute or chronic), pulmonary fibrosis, interstitial lung disease or hypersensitivity pneumonitis. In embodiments, the disease or disorder is an acute lung injury (ALI). In embodiments, the ALI is a pulmonary disorder that can be induced directly by inhalation of chemicals (chemical induced acute lung injury) or other means (e.g., infection) or can be induced indirectly by systemic injury (e.g., infection). Acute lung injury includes subcategories of respiratory distress syndromes including infant respiratory distress syndrome (IRDS), hyaline membrane disease (HMD), neonatal respiratory distress syndrome (NRDS), respiratory distress syndrome of newborn (RDSN), surfactant deficiency disorder (SDD), acute respiratory distress syndrome (ARDS), respiratory complication from systemic inflammatory response syndrome (SIRS), or severe acute respiratory syndrome (SARS).

In embodiments, the present invention relates to the therapeutic use of the present cells for the treatment of one or more symptoms associated with a viral infection.

In embodiments, the composition is suitable for use in the treatment of an infectious disease, optionally selected from an infection with a pathogen, optionally selected from a bacterium, virus, fungus, or parasite.

In embodiments, the pathogen is a virus. In embodiments, the virus is: (a) an influenza virus, optionally selected from Type A, Type B, Type C, and Type D influenza viruses, or (b) a member of the Coronaviridae family, optionally selected from (i) a betacoronavirus, optionally selected from Severe acute respiratory syndrome coronavirus 2 (SARS-CoV-2), SARS-CoV, Middle East Respiratory Syndrome-Corona Virus (MERS-CoV), HCoV-HKU1, and HCoV-OC43 or (ii) an alphacoronavirus, optionally selected from HCoV-NL63 and HCoV-229E.

In embodiments, the virus is SARS-CoV-2. In embodiments, the virus is SARS-CoV-2, which has caused COVID-19. In embodiments, the COVID-19 is characterized by one or more of fever, cough, shortness of breath, diarrhea, upper respiratory symptoms, lower respiratory symptoms, pneumonia, and respiratory distress.

In some embodiments, the composition is suitable for use in the treatment of an infection, wherein the infection is a coronavirus infection. In some embodiments, the coronavirus infection is one or more of severe acute respiratory syndrome coronavirus 2 (SARS-CoV-2), SARS-CoV, Middle East Respiratory Syndrome-Corona Virus (MERS-CoV), HCoV-HKU1, HCoV-OC43, HCoV-NL63, and HCoV-229E. In various embodiments, the coronavirus infection is SARS or COVID-19. In further embodiments, the subject is infected by SARS-CoV-2.

In embodiments, the therapy prevents or mitigates development of acute respiratory distress syndrome (ARDS) in a patient when administered. In embodiments, the therapy improves oxygenation in a patient when administered. In embodiments, the therapy improves systemic blood pressure oxygenation in a patient when administered, e.g., reducing or mitigating shock, e.g., requiring less pressor support. In embodiments, the therapy improves lung and/or alveolar permeability in a patient when administered.

In embodiments, the therapy prevents or mitigates a transition from respiratory distress to cytokine imbalance in a patient when administered. In embodiments, the therapy reverses or prevents a cytokine storm in a patient when administered. In embodiments, the therapy reverses or prevents a cytokine storm in the lungs or systemically in a patient when administered. In embodiments, the cytokine storm is selected from one or more of systemic inflammatory response syndrome, cytokine release syndrome, macrophage activation syndrome, and hemophagocytic lymphohistiocytosis.

In embodiments, the therapy reverses or prevents excessive production of one or more inflammatory cytokines in a patient when administered. In embodiments, the inflammatory cytokine is one or more of IL-6, IL-1, IL-1 receptor antagonist (IL-1ra), IL-2ra, IL-10, IL-18, TNFα, interferon-γ, CXCL10, and CCL7.

In embodiments, the present invention relates to the therapeutic use of the present cells for the treatment of one or more symptoms associated with a coronavirus infection.

Coronaviruses (CoVs) are members of the family Coronaviridae, including betacoronavirus and alphacoronavirus-respiratory pathogens that have relatively recently become known to invade humans. The Coronaviridae family includes such betacoronavirus as severe acute respiratory syndrome coronavirus 2 (SARS-CoV-2), SARS-CoV, Middle East Respiratory Syndrome-Corona Virus (MERS-CoV), HCoV-HKU1, and HCoV-OC43. Alphacoronavirus includes, e.g., HCoV-NL63 and HCoV-229E. In embodiments, the present invention relates to the therapeutic use of the present cells for the treatment of one or more symptoms of infection with any of severe acute respiratory syndrome coronavirus 2 (SARS-CoV-2), SARS-CoV, Middle East Respiratory Syndrome-Corona Virus (MERS-CoV), HCoV-HKU1, and HCoV-OC43. Alphacoronavirus includes, e.g., HCoV-NL63 and HCoV-229E.

Without wishing to be bound by theory, coronaviruses invade cells through utilization of their “spike” surface glycoprotein that is responsible for viral recognition of Angiotensin Converting Enzyme 2 (ACE2), a transmembrane receptor on mammalian hosts that facilitate viral entrance into host cells. (Zhou et al., A pneumonia outbreak associated with a new coronavirus of probable bat origin, Nature 2020).

Symptoms associated with coronavirus infections include, but are not limited to, fever, tiredness, dry cough, aches and pains, shortness of breath and other breathing difficulties, diarrhea, upper respiratory symptoms (e.g., sneezing, runny nose, nasal congestion, cough, sore throat), and/or pneumonia. In embodiments, the present compositions and methods are useful in treating or mitigating any of these symptoms.

In embodiments, the present invention relates to the therapeutic use of the present cells for the treatment of one or more symptoms of infection with SARS-CoV-2, including Coronavirus infection 2019 (COVID-19), caused by SARS-CoV-2 (e.g., 2019-nCoV).

In some settings, including subjects afflicted with coronavirus infections, it is possible that the morbidity and mortality of pulmonary viral infection is related to an exaggerated or overwhelming inflammatory response. In varying clinical circumstances this response can be described as “cytokine response syndrome”, “cytokine storm”, or “secondary hemophagocytic lymphohistiocytosis” (sHLH). In embodiments, the present compositions and methods are useful in treating or mitigating any of these exaggerated or overwhelming inflammatory responses. Collectively it is surmised that these highly proinflammatory states can lead to death due to pulmonary collapse such as acute respiratory distress syndrome (ARDS) or systemic, multi-organ failure affecting organs such as liver, kidney, heart and brain. In embodiments, the present cells treat or mitigate a “cytokine response syndrome”, “cytokine storm”, or “secondary hemophagocytic lymphohistiocytosis” (sHLH).

In embodiments, COVID-19 is characterized, in part, by elevation of Interleukin-2 (IL-2), Interleukin-7 (IL-7), granulocyte colony stimulating factor (GCSF), interferon-gamma inducible protein 10, monocyte chemoattractant protein-1 (MCP-1), macrophage inflammatory protein 1-alpha (MIP1a), and tumor necrosis factor-alpha (TNFα). In embodiments, the present compositions and methods are useful in treating or mitigating increases of any of these factors.

In embodiments, the present cells prevent a COVID-19 patient from having a disease that develops from respiratory distress to cytokine storm.

In embodiments, the present cells treat or mitigate ARDS.

In some embodiments, a cytokine storm is associated with COVID-19 and is treated or mitigated via a method comprising administering to a subject in need thereof an effective amount of cells effective for the treatment of a coronavirus infection and/or a cytokine storm associated with a coronavirus infection, wherein the subject has abnormal (e.g. increased or decreased) expression or activity of one or more of IL-6, IL-1, TNF, interferon-γ, CXCL10, CCL7, IL-1 receptor antagonist (IL-1ra), IL-2ra, IL-10, IL-18, CCL2/MCP-1, CCL5/RANTES, CCL7/MCP-3, MCP-2, tumor necrosis factor-alpha (TNFα), interferon-γ (IFNγ), CXCL10, CXC3, Granulocyte colony stimulatory factor (GCSF), Macrophage inflammatory protein 1 alpha (MIP-1a), IL-22, and Interferon gamma induced protein 10 (IP-10).

In some embodiments, the subject has a modulated (e.g. decreased or increased) expression or activity of one or more of IL-6, IL-1, TNF, interferon-γ, CXCL10, CCL7, IL-1 receptor antagonist (IL-1ra), IL-2ra, IL-10, IL-18, CCL2/MCP-1, CCL5/RANTES, CCL7/MCP-3, MCP-2, tumor necrosis factor-alpha (TNFα), interferon-γ (IFNγ), CXCL10, CXC3, Granulocyte colony stimulatory factor (GCSF), Macrophage inflammatory protein 1 alpha (MIP-1a), IL-22, and Interferon gamma induced protein 10 (IP-10).

In embodiments, the disease/indication is associated with one or more cancers. The one or more cancers may comprise: adenoid cystic carcinoma, adrenal gland tumor, amyloidosis, anal cancer, appendix cancer, astrocytoma, ataxia-telangiectasia, Beckwith-Wiedemann Syndrome, bile duct caner (Cholangiocarcinoma), Birt-Hogg Dube Syndrome, bladder cancer, bone cancer (sarcoma of bone), brain stem glioma, brain tumor, breast cancer, breast cancer (inflammatory), breast cancer (metastatic), breast cancer in men, carney complex, central nervous system tumors (brain and spinal cord), cervical cancer, childhood cancer, colorectal cancer, Cowden Syndrome, craniopharyngioma, desmoid tumor, desmoplastic infantile ganglioglioma tumor, ependymoma, esophageal cancer, Ewing Sarcoma, eye cancer, eyelid cancer, familial adenomatous polyposis, familial GIST, familial malignant melanoma, familial pancreatic cancer, gallbladder cancer, gastrointestinal stromal tumor GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, HIV/AIDS related cancer, juvenile polyposis syndrome, kidney cancer, lacrimal gland tumor, laryngeal and hypopharyngeal cancer, leukemia—acute lymphoblastic—ALL, leukemia, acute lymphocytic—ALL, leukemia—acute myeloid—ALL, leukemia—acute myeloid—AML, leukemia—B-cell prolymphocytic leukemia and hairy cell leukemia, leukemia—chronic lymphocytic—CLL, leukemia—chronic myeloid—CML, leukemia—chronic t-cell lymphocytic, leukemia—eosinophilic, Li—Fraumeni Syndrome, liver cancer, lung cancer—non-small cell, lung cancer—small cell, lymphoma—Hodgkin, lymphoma—Non-Hodgkin, Lynch Syndrome, mastocytosis, medulloblastoma, melanoma, meningioma, mesothelioma, multiple endocrine neoplasia type 1, multiple endocrine neoplasia type 2, multiple myeloma, MUTYH/MYH—associated polyposis, myelodysplastic syndromes—MDS, nasal cavity and paranasal sinus cancer, nasopharyngeal cancer, neuroblastoma, neuroendocrine tumor of the gastrointestinal tract, neuroendocrine tumor of the lung, neuroendocrine tumor of the pancreas, neuroendocrine tumors, neurofibromatosis type 1, neurofibromatosis type 2, nevoid basal cell carcinoma syndrome, oral and oropharyngeal cancer, osteosarcoma, ovarian cancer, fallopian tube cancer, peritoneal cancer, pancreatic cancer, parathyroid cancer, penile cancer, Peutz-Jeghers Syndrome, pheochromocytoma and paraganglioma, pituitary gland tumor, pleuropulmonary blastoma, prostate cancer, retinoblastoma, rhabdomyosarcoma, salivary gland cancer, sarcoma—Kaposi, sarcomas of specific organs, sarcomas—soft tissue, skin cancer (non-melanoma), skin cancer (melanoma), small bowel cancer, stomach cancer, testicular cancer, thyoma and thymic carcinoma, thyroid cancer, tuberous sclerosis complex, unknown primary, uterine cancer, vaginal cancer, Von Hippel-Lindau Syndrome, vulvar cancer, Waldenstrom macroglobulinemia (lymphoplasmacytic lymphoma), Werner Syndrome, Wilms tumor, and xeroderma pigmentosum.

In an aspect, the present disclosure provides a method for treating a cancer in a patient in need thereof. The method comprising administering to the cancer patient a therapeutically-effective amounts of any herein-disclosed cytotoxic lymphocyte.

An aspect of the present disclosure is a method for killing a cancer cell. The method comprising steps of (1) obtaining a herein-disclosed cytotoxic lymphocyte and (2) contacting cytotoxic lymphocyte with the cancer. In some cases, the cancer cell is in vivo.

Yet another aspect of the present disclosure is a method for treating a cancer patient in need thereof. The method comprising a step of administering to the cancer patient a therapeutically-effective amounts of a herein-disclosed cytotoxic lymphocyte.

In aspects, the present disclosure provides a method of treating cancer, comprising: (a) obtaining an isolated cytotoxic lymphocyte comprising a genetically engineered disruption in a beta-2-microglobulin (B2M) gene; and (b) administering the isolated cytotoxic lymphocyte to a subject in need thereof.

In some cases, the lymphoid lineage cell is a T cell, e.g., a cytotoxic T cell or gamma-delta T cell; an NK cell; or an NK-T cell.

In some cases, the myeloid lineage cell is a macrophage, e.g., an M1 macrophage or an M2 macrophage.

In embodiments, the cytotoxic lymphocyte is an NK cell.

In embodiments, the cancer is a blood cancer. In embodiments, the cancer is a solid tumor. In embodiments, the cancer is selected from basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g., that associated with brain tumors), and Meigs' syndrome.

The cytotoxic lymphocyte of the present disclosure may be administered systemically (e.g., via a vein or artery) or may be introduced into a tumor or in the vicinity of the tumor.

### Administration and Formulations

In some embodiments, the present disclosure relates to compositions described herein in the form of a pharmaceutical composition.

An aspect of the present disclosure is a method for treating a disease or disorder, e.g., cancer.

Therapeutic treatments comprise the use of one or more routes of administration and of one or more formulations that are designed to achieve a therapeutic effect at an effective dose, while minimizing toxicity to the subject to which treatment is administered. Illustrative formulations/compositions of the present disclosure include engineered cells along with a suitable delivery reagent, e.g., a liquid carrier.

In various embodiments, the effective dose is an amount that substantially avoids cell toxicity in vivo. In various embodiments, the effective dose is an amount that substantially avoids an immune reaction in a human subject. For example, the immune reaction may be an immune response mediated by the innate immune system. Immune response can be monitored using markers known in the art (e.g., cytokines, interferons, TLRs). In some embodiments, the effective dose obviates the need for treatment of the human subject with immune suppressants agents (e.g., B18R) used to moderate the residual toxicity.

Upon formulation, solutions may be administered in a manner compatible with the dosage formulation and in such amount as is therapeutically effective, as described herein. The formulations may easily be administered in a variety of dosage forms such as injectable solutions and the like. For parenteral administration in an aqueous solution, for example, the solution generally is suitably buffered, and the liquid diluent first rendered isotonic with, for example, sufficient saline or glucose. Such aqueous solutions may be used, for example, for intravenous, intramuscular, subcutaneous and intraperitoneal administration. Preferably, sterile aqueous media are employed as is known to those of skill in the art.

Pharmaceutical preparations may additionally comprise delivery reagents (a.k.a. “vehicles”, a.k.a. “delivery vehicles”) and/or excipients. Pharmaceutically acceptable delivery reagents, excipients, and methods of preparation and use thereof, including methods for preparing and administering pharmaceutical preparations to patients (a.k.a. “subjects”) are well known in the art, and are set forth in numerous publications, including, for example, in US Patent Appl. Pub. No. US 2008/0213377, the entirety of which is incorporated herein by reference.

The present pharmaceutical compositions can comprise excipients, including liquids such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the composition is formulated for one or more of intrathecal, intra-lesional, intra-coronary, intravenous (IV), intra-articular, intramuscular, and intra-endobronchial administration and administration via intrapancreatic endovascular injection, intra-nucleus pulposus, lumbar puncture, intra-myocardium, transendocardium, intra-fistula tract, intermedullary space, intradural space and leg injection.

In embodiments, the composition is formulated for infusion. In some embodiments, the composition is formulated for infusion, wherein the composition is delivered to the bloodstream of a subject or patient through a needle in a vein of the subject or patient through a peripheral line, a central line, a tunneled line, an implantable port, and/or a catheter. In some embodiments, the subject or patient may also receive supportive medications or treatments, such as hydration, by infusion. In some embodiments, the composition is formulated for intravenous infusion. In some embodiments, the infusion is continuous infusion, secondary intravenous therapy (IV), and/or IV push. In some embodiments, the infusion of the composition may be administered through the use of equipment selected from one or more of an infusion pump, hypodermic needle, drip chamber, peripheral cannula, and pressure bag.

In embodiments, the method of treating a subject comprises administering a cell of the present disclosure to a subject in need thereof. In embodiments, the cell is formulated for therapeutic use. In embodiments, the cell is suitable for administration to a human subject. In embodiments, the method is conducted in vivo.

In numerous embodiments, the administering is intravenous, intraarterial, intratumoral, or injected in the vicinity of a tumor.

In embodiments when a synthetic RNA molecule encoding a temperature-sensitive gene-editing protein is administered, the method comprises reducing the body temperature of a subject, optionally via whole-body hypothermia. In embodiments, the body temperature of the subject is reduced by from about 0.5° C. to about 1° C. In embodiments, the body temperature of the subject is reduced by from about 1° C. to about 1.5° C. In embodiments, the body temperature of the subject is reduced by from about 1.5° C. to about 2° C. In embodiments, the body temperature of the subject is reduced by from about 2° C. to about 2.5° C. In embodiments, the body temperature of the subject is reduced by from about 2.5° C. to about 3° C. In embodiments, the body temperature of the subject is reduced by from about 3° C. to about 3.5° C. In embodiments, the body temperature of the subject is reduced by from about 3.5° C. to about 4° C. In embodiments, the body temperature of the subject is reduced by from about 4° C. to about 4.5° C. In embodiments, the body temperature of the subject is reduced by from about 4.5° C. to about 5° C. In embodiments, the body temperature of the subject is reduced by from about 5° C. to about 5.5° C. In embodiments, the body temperature of the subject is reduced by from about 5.5° C. to about 6° C. In embodiments, the body temperature of the subject is reduced by from about 6° C. to about 6.5° C. In embodiments, the body temperature of the subject is reduced by from about 6.5° C. to about 7° C. In embodiments, the body temperature of the subject is reduced by from about 7° C. to about 7.5° C. In embodiments, the body temperature of the subject is reduced by from about 7.5° C. to about 8° C. In embodiments, the body temperature of the subject is reduced by from about 8° C. to about 8.5° C. In embodiments, the body temperature of the subject is reduced by from about 8.5° C. to about 9° C. In embodiments, the body temperature of the subject is reduced by from about 9° C. to about 9.5° C. In embodiments, the body temperature of the subject is reduced by from about 9.5° C. to about 10° C. In embodiments, the body temperature of the subject is reduced by from about 10° C. to about 10.5° C. In embodiments, the body temperature of the subject is reduced by from about 10.5° C. to about 11° C. In embodiments, the body temperature of the subject is reduced by from about 11° C. to about 11 0.5° C. In some embodiments, the reducing the body temperature of the subject is performed for a specific amount of time. In some embodiments, the specific amount of time is from about 15 minutes to about 30 minutes. In some embodiments, the specific amount of time is from about 30 minutes to about 45 minutes. In some embodiments, the specific amount of time is from about 45 minutes to about 60 minutes. In embodiments, the specific amount of time is from about 1 hour to about 1.5 hours. In embodiments, the specific amount of time is from about 1.5 hours to about 2 hours. In embodiments, the specific amount of time is from about 2 hours to about 2.5 hours. In embodiments, the specific amount of time is from about 2.5 hours to about 3 hours. In embodiments, the specific amount of time is from about 3 hours to about 3.5 hours. In embodiments, the specific amount of time is from about 3.5 hours to about 4 hours. In embodiments, the specific amount of time is from about 4 hours to about 4.5 hours. In embodiments, the specific amount of time is from about 4.5 hours to about 5 hours. In embodiments, the specific amount of time is from about 5 hours to about 5.5 hours. In embodiments, the specific amount of time is from about 5.5 hours to about 6 hours. In embodiments, the specific amount of time is from about 6 hours to about 6.5 hours.

In embodiments when a synthetic RNA molecule encoding a temperature-sensitive gene-editing protein is administered, the method comprises applying one or more cooling elements to a cell or tissue in vivo to reduce temperature, the cooling element optionally being a cryocompression device. In embodiments, the temperature is reduced by from about 0.5° C. to about 1° C. In embodiments, the temperature is reduced by from about 1° C. to about 1.5° C. In embodiments, the temperature is reduced by from about 1.5° C. to about 2° C. In embodiments, the temperature is reduced by from about 2° C. to about 2.5° C. In embodiments, the temperature is reduced by from about 2.5° C. to about 3° C. In embodiments, the temperature is reduced by from about 3° C. to about 3.5° C. In embodiments, the temperature is reduced by from about 3.5° C. to about 4° C. In embodiments, the temperature is reduced by from about 4° C. to about 4.5° C. In embodiments, the temperature is reduced by from about 4.5° C. to about 5° C. In embodiments, the temperature is reduced by from about 5° C. to about 5.5° C. In embodiments, the temperature is reduced by from about 5.5° C. to about 6° C. In embodiments, the temperature is reduced by from about 6° C. to about 6.5° C. In embodiments, the temperature is reduced by from about 6.5° C. to about 7° C. In embodiments, the temperature is reduced by from about 7° C. to about 7.5° C. In embodiments, the temperature is reduced by from about 7.5° C. to about 8° C. In embodiments, the temperature is reduced by from about 8° C. to about 8.5° C. In embodiments, the temperature is reduced by from about 8.5° C. to about 9° C. In embodiments, the temperature is reduced by from about 9° C. to about 9.5° C. In embodiments, the temperature is reduced by from about 9.5° C. to about 10° C. In embodiments, the temperature is reduced by from about 10° C. to about 10.5° C. In embodiments, the temperature is reduced by from about 10.5° C. to about 11° C. In embodiments, the temperature is reduced by from about 11° C. to about 11.5° C.

In embodiments when a synthetic RNA molecule encoding a temperature-sensitive gene-editing protein is administered, the applying one or more cooling elements to a cell or tissue in vivo to reduce temperature is performed for a specific amount of time. In some embodiments, the specific amount of time is from about 15 minutes to about 30 minutes. In some embodiments, the specific amount of time is from about 30 minutes to about 45 minutes. In some embodiments, the specific amount of time is from about 45 minutes to about 60 minutes. In some embodiments, the specific amount of time is from about 1 hour to about 1.5 hours. In some embodiments, the specific amount of time is from about 1.5 hours to about 2 hours. In some embodiments, the specific amount of time is from about 2 hours to about 2.5 hours. In some embodiments, the specific amount of time is from about 2.5 hours to about 3 hours. In some embodiments, the specific amount of time is from about 3 hours to about 3.5 hours. In some embodiments, the specific amount of time is from about 3.5 hours to about 4 hours. In some embodiments, the specific amount of time is from about 4 hours to about 4.5 hours. In some embodiments, the specific amount of time is from about 4.5 hours to about 5 hours. In some embodiments, the specific amount of time is from about 5 hours to about 5.5 hours. In some embodiments, the specific amount of time is from about 5.5 hours to about 6 hours. In some embodiments, the specific amount of time is from about 6 hours to about 6.5 hours.

Further description of temperature-sensitive cell administration is found in US20230193231A1. The entire contents of which are incorporated by reference in their entirety.

In some embodiments, the present invention relates to one or more administration techniques described in U.S. Pat. Nos. 5,711,964; 5,891,468; 6,316,260; 6,413,544; 6,770,291; and 7,390,780, the entire contents of which are hereby incorporated by reference in their entireties.

### Lipids/Cell Contacting/Transfection

In embodiments, the present invention relates delivery of the present synthetic RNA molecules via a lipid. In some embodiments, mRNAs encoding a gene-editing protein and/or a reprogramming factor are delivered via a lipid.

In embodiments, the lipid is a compound of Formula (I)

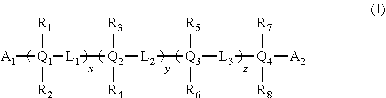

wherein: Q₁, Q₂, Q₃, and Q₄ are independently an atom or group of atoms capable of adopting a positive charge;
A₁ and A₂ are independently null, H, or optionally substituted C₁-C₆ alkyl;
L₁, L₂, and L₃ are independently null, a bond, (C₁-C₂₀)alkanediyl, (halo)(C₁-C₂₀)alkanediyl, (hydroxy)(C₁-C₂₀)alkanediyl, (alkoxy)(C₁-C₂₀)alkanediyl, arylene, heteroarylene, cycloalkanediyl, heterocycle-diyl, or any combination of the aforementioned optionally linked by one or more of an ether, an ester, an anhydride, an amide, a carbamate, a secondary amine, a tertiary amine, a quaternary ammonium, a thioether, a urea, a carbonyl, or an imine;
R₁, R₂, R₃, R₄, R₅, R₆, R₇, and R₈ are independently null, H, (C₁-C₆₀)alkyl, (halo)(C₁-C₆₀)alkyl, (hydroxy)(C₁-C₆₀)alkyl, (alkoxy)(C₁-C₆₀)alkyl, (C₂-C₆₀)alkenyl, (halo)(C₂-C₆₀)alkenyl, (hydroxy)(C₂-C₆₀)alkenyl, (alkoxy)(C₂-C₆₀)alkenyl, (C₂-C₆₀)alkynyl, (halo)(C₂-C₆₀)alkynyl, (hydroxy)(C₂-C₆₀)alkynyl, (alkoxy)(C₂-C₆₀)alkynyl, wherein at least one of R₁, R₂, R3, R4, R5, R6, R7, and R8 comprises at least two unsaturated bonds; and x, y, and z are independently 0, 1, 2, 3, 4, 5, 6, 7, 8, 9, 10, 11, 12, 13, 14, or 15.

In embodiments, the lipid is a compound of Formula (II):

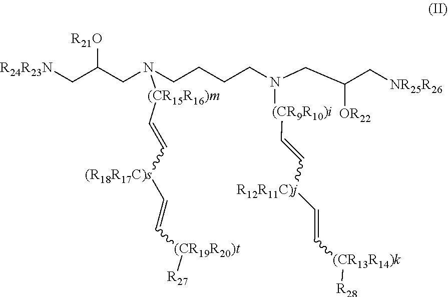

wherein: R9, R10, R11, R12, R13, R14, R15, R16, R17, R18, R19, R20, R21, R22, R23, R24, R25, R26, R27, and R28 are independently H, halo, OH, (C1-C6)alkyl, (halo)(C1-C6)alkyl, (hydroxy)(C1-C6)alkyl, (alkoxy)(C1-C6)alkyl, aryl, heteroaryl, cycloalkyl, or heterocyclo; and i, j, k, m, s, and t are independently 0, 1, 2, 3, 4, 5, 6, 7, 8, 9, 10, 11, 12, 13, 14, or 15.

In embodiments, the lipid is a compound of Formula (III):

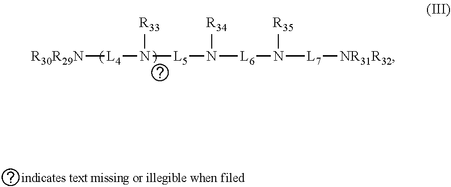

wherein L₄, L₅, L₆, and L₇ are independently a bond, (C₁-C₂₀)alkanediyl, (halo)(C₁-C₂₀)alkanediyl, (hydroxy)(C₁-C₂₀)alkanediyl, (alkoxy)(C₁-C₂₀)alkanediyl, arylene, heteroarylene, cycloalkanediyl, heterocycle-diyl, —(CH₂)ᵥ₁—C(O)—, —((CH₂)ᵥ₁—O)ᵥ₂—, or —((CH₂)ᵥ₁—C(O)—O)ᵥ₂—;
R₂₉, R₃₀, R₃₁, R₃₂, R₃₃, R₃₄, and R₃₅ are independently H, (C₁-C₆₀)alkyl, (halo)(C₁-C₆₀)alkyl, (hydroxy)(C₁-C₆₀)alkyl, (alkoxy)(C₁-C₆₀)alkyl, (C₂-C₆₀)alkenyl, (halo)(C₂-C₆₀)alkenyl, (hydroxy)(C₂-C₆₀)alkenyl, (alkoxy)(C₂-C₆₀)alkenyl, (C₂-C₆₀)alkynyl, (halo)(C₂-C₆₀)alkynyl, (hydroxy)(C₂-C₆₀)alkynyl, (alkoxy)(C₂-C₆₀)alkynyl, wherein at least one of R₂₉, R₃₀, R₃₁, R₃₂, R₃₃, R₃₄, and R₃₅ comprises at least two unsaturated bonds;
v, v₁ and v₂ are independently 0, 1, 2, 3, 4, 5, 6, 7, 8, 9, 10, 11, 12, 13, 14, or 15.

In embodiments, the lipid is a compound of Formula (IV):

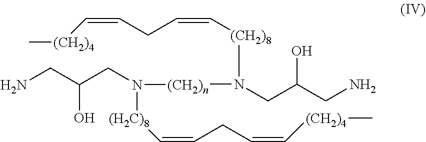

wherein n is 1, 2, 3, 4, 5, 6, 7, 8, 9, 10, 11, 12, 13, 14, or 15.

In embodiments, the lipid is a compound of Formula (V):

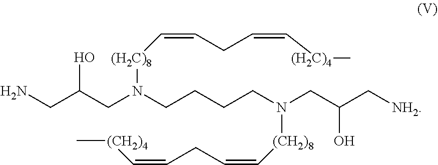

In embodiments, the lipid is a compound of Formula (VI):

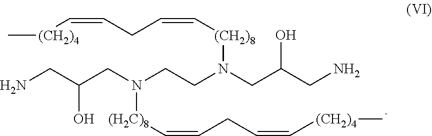

In embodiments, the lipid is a compound of Formula (VII):

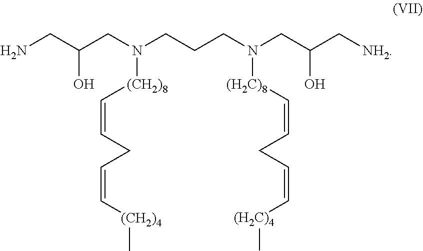

In embodiments, the lipid is a compound of Formula (VIII):

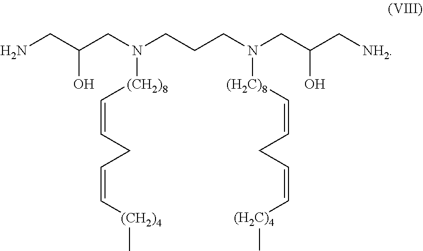

In embodiments, the lipid is a compound of Formula (IX):

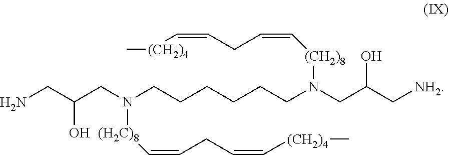

In embodiments, the lipid is a compound of Formula (X):

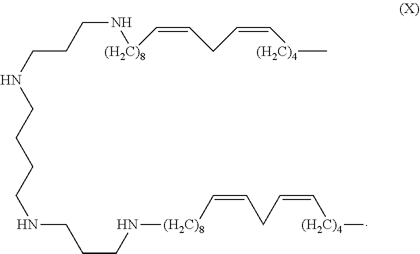

In embodiments, the lipid is a compound of Formula (XI):

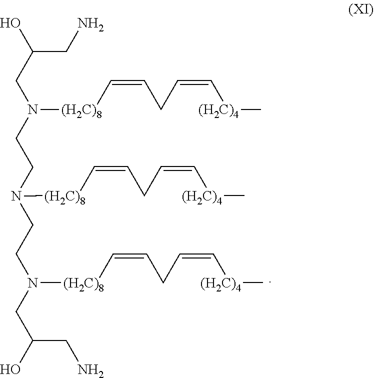

In embodiments, the lipid is a compound of Formula (XII):

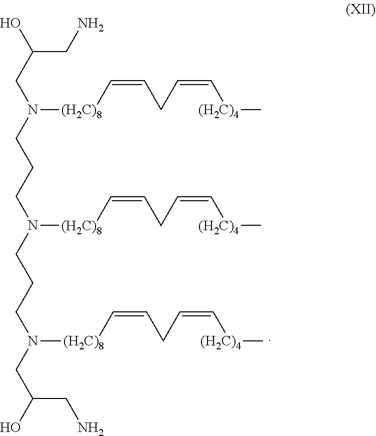

In embodiments, the lipid is a compound of Formula (XIII):

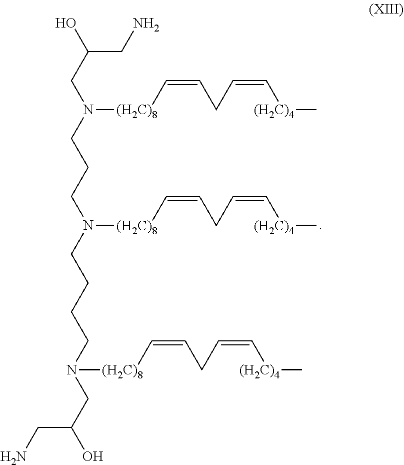

In embodiments, the lipid is a compound of Formula (XIV):

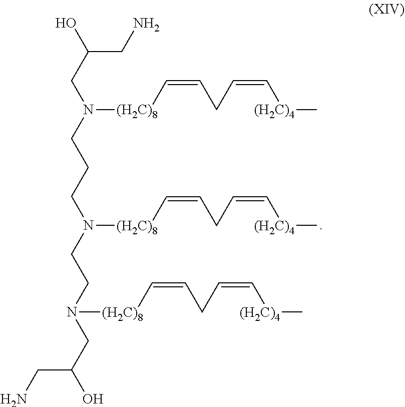

In embodiments, the lipid is a compound of Formula (XV):

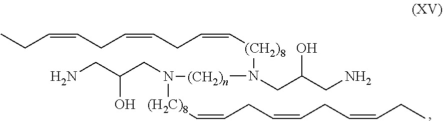

wherein n is 1, 2, 3, 4, 5, 6, 7, 8, 9, 10, 11, 12, 13, 14, or 15.

In embodiments, the lipid is a compound of Formula (XVI):

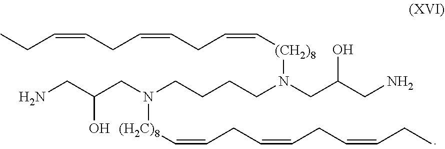

In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle.

In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle which does not require an additional or helper lipid. In embodiments, the present compounds (e.g., of Formulae I-XVI) are components of a pharmaceutical composition and/or a lipid aggregate and/or a lipid carrier and/or a lipid nucleic-acid complex and/or a liposome and/or a lipid nanoparticle that further comprises a neutral lipid (e.g., dioleoylphosphatidylethanolamine (DOPE), 1,2-Dioleoyl-sn-glycero-3-phosphocholine (DOPC), or cholesterol) and/or a further cationic lipid (e.g., N-[1-(2,3-dioleoyloxy)propyl]-N,N,N-trimethylammonium chloride (DOTMA), 1,2-bis(oleoyloxy)-3-3-(trimethylammonium) propane (DOTAP), or 1,2-dioleoyl-3-dimethylammonium-propane (DODAP)). 05841 In-embodiments, the lipid is any of those described in International Patent Publication No. WO 2021/003462, hereby incorporated by reference in its entirety.

In embodiments, the lipid is any of those of Table 7.

TABLE 7
Illustrative Biocompatible Lipids and Polymers

3β -[N-(N′,N′-dimethylaminoethane)-carbamoyl]cholesterol (DC-Cholesterol)
1,2-dioleoyl-3-trimethylammonium-propane (DOTAP/18:1 TAP)
N-(4-carboxybenzyl)-N,N-dimethyl-2,3-bis(oleoyloxy)propan-1-aminium (DOBAQ)
1,2-dimyristoyl-3-trimethylammonium-propane (14:0 TAP)
1,2-dipalmitoyl-3-trimethylammonium-propane (16:0 TAP)
1,2-stearoyl-3-trimethylammonium-propane (18:0 TAP)
1,2-dioleoyl-3-dimethylammonium-propane (DODAP/18:1 DAP)
1,2-dimyristoyl-3-dimethylammonium-propane (14:0 DAP)
1,2-dipalmitoyl-3-dimethylammonium-propane (16:0 DAP)
1,2-distearoyl-3-dimethylammonium-propane (18:0 DAP)
dimethyldioctadecylammonium (18:0 DDAB)
1,2-dilauroyl-sn-glycero-3-ethylphosphocholine (12:0 EthylPC)
1,2-dimyristoyl-sn-glycero-3-ethylphosphocholine (14:0 EthylPC)
1,2-dimyristoleoyl-sn-glycero-3-ethylphosphocholine (14:1 EthylPC)
1,2-dipalmitoyl-sn-glycero-3-ethylphosphocholine (16:0 EthylPC)
1,2-distearoyl-sn-glycero-3-ethylphosphocholine (18:0 EthylPC)
1,2-dioleoyl-sn-glycero-3-ethylphosphocholine (18:1 EthylPC)
1-palmitoyl-2-oleoyl-sn-glycero-3-ethylphosphocholine (16:1-18:1 EthylPC)
1,2-di-O-octadecenyl-3-trimethylammonium propane (DOTMA)
N1-[2-((1S)-1-[(3-aminopropyl)amino]-4-[di(3-amino-
propyl)amino]butylcarboxamido)ethyl]-3,4-di[oleyloxy]-benzamide (MVL5)
2,3-dioleyloxy-N-[2-spermine carboxamide]ethyl-N,N-dimethyl-1-propanammonium
trifluoroacetate (DOSPA)
1,3-di-oleoyloxy-2-(6-carboxy-spermyl)-propylamid (DOSPER)
N-[1-(2,3-dimyristyloxy)propyl]-N,N-dimethyl-N-(2-hydroxyethyl)ammonium
bromide (DMRIE)
LIPOFECTAMINE, LIPOFECTAMINE 2000, LIPOFECTAMINE RNAiMAX,
LIPOFECTAMINE 3000, LIPOFECTAMINE MessengerMAX, TransIT mRNA
dioctadecyl amidoglyceryl spermine (DOGS)
dioleoyl phosphatidyl ethanolamine (DOPE)
1,2-dilinoleyloxy-3-dimethylaminopropane (DLinDMA)
1,2-dilinoleyl-4-(2-dimethylaminoethyl)-[1,3]-dioxolane (DLin-KC2-DMA)
Heptatriaconta-6,9,28,31-tetraen-19-yl 4-(dimethylamino)butanoate (DLin-MC3-
DMA)
N1,N4-dimyristyl-N1,N4-di-(2-hydroxy-3-aminopropyl)-diaminobutane (DHDMS)
N1,N4-dioleyl-N1,N4-di-(2-hydroxy-3-aminopropyl)-diaminobutane (DHDOS)
1,2-distearoyl-sn-glycero-3-phosphocholine (18:0 PC DSPC)
1,2-dioleyl-sn-glycero-3-phosphocholine (18:1 PC)
1,2-distearyl-sn-glycero-3-phosphatidyl ethanolamine (DSPE)
1,2-dilinoleyl-3-dimethylammonium-propane (18:2 DAP)
hexadimethrine bromide (Polybrene ™)
DEAE-Dextran
protamine
protamine sulfate
poly-L-lysine
poly-D-lysine
Poly(beta-amino-ester) polymer
polyethyleneimine
block co-polymer comprising one or more of: PEG, PLGA, PPG, PEI, PLL, PCL,
a PLURONIC

Protein Expression and/or Secretion Signature

Each type of cell expresses particular sets of proteins, within the cell, on the cell's surface, and secreted into the extracellular space. The particular sets of proteins that each type of cell expresses depends on the general and immediate function of the cell. Protein expression is correlated with mRNA levels and thus can be assayed by methods that analyze the distribution, amount, and identity of particular mRNAs within a cell. There are several methods of quantitatively measuring mRNA, including northern blotting and reverse transcription-quantitative PCR (RT-qPCR). Hybridization microarrays may also be used to generate expression profiles or high-throughput analyses of a range of genes within a cell. Further, ‘tag based’ technologies, such as Serial analysis of gene expression (SAGE) and RNA-Seq can be used to determine the relative measure of the cellular concentration of different mRNAs.

In some embodiments, protein expression of specific cells is determined by determining concentration of different mRNAs by one or more of northern blotting, RT-qPCR, hybridization microarrays, and tag-based technologies, such as SAGE and RNA-Seq.

There are generally two strategies used for detection of proteins in the extracellular milieu: direct methods and indirect methods. The direct method comprises a one-step staining, and may involve a labeled antibody (e.g., FITC conjugated antiserum) reacting directly with the protein in the extracellular milieu. The indirect method comprises an unlabeled primary antibody that reacts with the protein in the extracellular milieu, and a labeled secondary antibody that reacts with the primary antibody. Labels can include radioactive labels, fluorescent labels, hapten labels such as, biotin, or an enzyme such as horse radish peroxidase or alkaline phosphatase. Methods of conducting these assays are well known in the art. See, e.g., Harlow et al. (Antibodies, Cold Spring Harbor Laboratory, NY, 1988), Harlow et al. (Using Antibodies, A Laboratory Manual, Cold Spring Harbor Laboratory, NY, 1999), Virella (Medical Immunology, 6th edition, Informa HealthCare, New York, 2007), and Diamandis et al. (Immunoassays, Academic Press, Inc., New York, 1996). Kits for conducting these assays are commercially available from, for example, Clontech Laboratories, LLC. (Mountain View, CA). In some embodiments, proteins are detected in the extracellular milieu of monocytes or macrophages using detection methods comprising one or more antibodies. In some embodiments, the detection methods further comprise labels, including radioactive labels, fluorescent labels, hapten labels such as, biotin, or an enzyme such as horse radish peroxidase or alkaline phosphatase.

In some embodiments, flow cytometry is used to determine whether cells express certain sets of proteins that are on the surface or that are secreted. In some embodiments, antibodies specific to particular proteins are used in combination with proteomic approaches to determine, e.g., the protein secretion signature of a particular cell. In some embodiments, the supernatant of a purified set of cell types is assayed using a Western blot to determine the concentrations of an array of secreted proteins, to which antibodies are available. In some embodiments, the protein secretion signatures of specific cell derived from different sources, such as iPSCs, skin cells, or bone marrow are determined and compared.

In some embodiments, iPSC-derived monocytes are characterized for expression of key hematopoietic and myeloid-lineage markers CD11b, CD13, CD14, CD33, CD45, CD80, CD163, CD206, and SIRPα. The expression of these makers iPSC-derived monocytes may be compared peripheral blood mononuclear cell (PBMC)-derived monocytes.

In some embodiments, the iPSC-derived monocytes show similar expression of CD11b, CD13, CD14, CD33, CD45, and CD163 compared to PBMC-derived monocytes, and increased expression of markers indicative of an activated state: CD80 and CD206.

In some embodiments, the iPSC-derived macrophages are characterized for expression of CD11b, CD68, CD80, CD86, CD163, CD206, and SIRPα and for secretion of, at least, the cytokines TNFα, IL-12p70, and IL-10. In embodiments, M1 and M2 polarized iPSC-derived macrophages secrete similar levels of TNFα, IL-12p70, and IL-10 compared to PBMC-derived macrophages.

In some embodiments, the iMSCs are characterized for expression of CD34, CD44, CD45, CD73, and CD90.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art. The terminology used herein is for the purpose of describing particular cases only and is not intended to be limiting.

As used herein, “a,” “an,” or “the” can mean one or more than one.

Herein the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Further, the term “about” when used in connection with a referenced numeric indication means the referenced numeric indication plus or minus up to 10% of that referenced numeric indication. For example, the language “about 50” covers the range of 45 to 55. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.” Although the open-ended term “comprising” is used herein to describe and claim the invention, the present invention, or embodiments thereof, may alternatively be described using alternative terms such as “consisting of” or “consisting essentially of.”

The term “substantially” is meant to be a significant extent, for the most part; or essentially. In other words, the term substantially may mean nearly exact to the desired attribute or slightly different from the exact attribute. Substantially may be indistinguishable from the desired attribute. Substantially may be distinguishable from the desired attribute but the difference is unimportant or negligible.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

By preventing is meant, at least, avoiding the occurrence of a disease and/or reducing the likelihood of acquiring the disease.

By treating is meant, at least, ameliorating or avoiding the effects of a disease, including reducing a sign or symptom of the disease.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

1]“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid or the like, which is nontoxic, and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

By “synthetic RNA molecule” is meant an RNA molecule that is produced outside of a cell or that is produced inside of a cell using bioengineering, by way of non-limiting example, an RNA molecule that is produced in an in vitro-transcription reaction, an RNA molecule that is produced by direct chemical synthesis or an RNA molecule that is produced in a genetically-engineered E. coli cell.

By “medium” is meant a solvent or a solution comprising a solvent and a solute, by way of non-limiting example, Dulbecco's Modified Eagle's Medium (DMEM), DMEM+10% fetal bovine serum (FBS), saline or water.

By “transfection medium” is meant a medium that can be used for transfection, by way of non-limiting example, Dulbecco's Modified Eagle's Medium (DMEM), DMEM/F12, saline or water.

By “Oct4 protein” is meant a protein that is encoded by the POU5F1 gene, or a natural or engineered variant, family-member, orthologue, fragment or fusion construct thereof, by way of non-limiting example, human Oct4 protein, mouse Oct4 protein, Oct1 protein, a protein encoded by POU5F1 pseudogene 2, a DNA-binding domain of Oct4 protein or an Oct4-GFP fusion protein. In some embodiments the Oct4 protein comprises an amino acid sequence that has at least 70% identity with SEQ ID NO: 76, or in other embodiments, at least 75%, 80%, 85%, 90%, or 95% identity with SEQ ID NO: 76. In some embodiments, the Oct4 protein comprises an amino acid sequence having from 1 to 20 amino acid insertions, deletions, or substitutions (collectively) with respect to SEQ ID NO: 76. Or in other embodiments, the Oct4 protein comprises an amino acid sequence having from 1 to 15 or from 1 to 10 amino acid insertions, deletions, or substitutions (collectively) with respect to SEQ ID NO: 76.

(SEQ ID NO: 76)
MAGHLASDFAFSPPPGGGGDGPGGPEPGWVDPRTWLSFQGPPGGPGIGPG
VGPGSEVWGIPPCPPPYEFCGGMAYCGPQVGVGLVPQGGLETSQPEGEAG
VGVESNSDGASPEPCTVTPGAVKLEKEKLEQNPEESQDIKALQKELEQFA
KLLKQKRITLGYTQADVGLTLGVLFGKVFSQTTICRFEALQLSFKNMCKL
RPLLQKWVEEADNNENLQEICKAETLVQARKRKRTSIENRVRGNLENLFL
QCPKPTLQQISHIAQQLGLEKDVVRVWFCNRRQKGKRSSSDYAQREDFEA
AGSPFSGGPVSFPLAPGPHFGTPGYGSPHFTALYSSVPFPEGEAFPPVSV
TTLGSPMHSN

By “Sox2 protein” is meant a protein that is encoded by the SOX2 gene, or a natural or engineered variant, family-member, orthologue, fragment or fusion construct thereof, by way of non-limiting example, human Sox2 protein, mouse Sox2 protein, a DNA-binding domain of Sox2 protein or a Sox2-GFP fusion protein. In some embodiments the Sox2 protein comprises an amino acid sequence that has at least 70% identity with SEQ ID NO: 77, or in other embodiments, at least 75%, 80%, 85%, 90%, or 95% identity with SEQ ID NO: 77. In some embodiments, the Sox2 protein comprises an amino acid sequence having from 1 to 20 amino acid insertions, deletions, or substitutions (collectively) with respect to SEQ ID NO: 77. Or in other embodiments, the Sox2 protein comprises an amino acid sequence having from 1 to 15 or from 1 to 10 amino acid insertions, deletions, or substitutions (collectively) with respect to SEQ ID NO: 77.

(SEQ ID NO: 77)
MYNMMETELKPPGPQQTSGGGGGNSTAAAAGGNQKNSPDRVKRPMNAFMV
WSRGQRRKMAQENPKMHNSEISKRLGAEWKLLSETEKRPFIDEAKRLRAL
HMKEHPDYKYRPRRKTKTLMKKDKYTLPGGLLAPGGNSMASGVGVGAGLG
AGVNQRMDSYAHMNGWSNGSYSMMQDQLGYPQHPGLNAHGAAQMQPMHRY
DVSALQYNSMTSSQTYMNGSPTYSMSYSQQGTPGMALGSMGSVVKSEASS
SPPVVTSSSHSRAPCQAGDLRDMISMYLPGAEVPEPAAPSRLHMSQHYQS
GPVPGTAINGTLPLSHM

By “Klf4 protein” is meant a protein that is encoded by the KLF4 gene, or a natural or engineered variant, family-member, orthologue, fragment or fusion construct thereof, by way of non-limiting example, human Klf4 protein, mouse Klf4 protein, a DNA-binding domain of Klf4 protein or a Klf4-GFP fusion protein. In some embodiments the Klf4 protein comprises an amino acid sequence that has at least 70% identity with SEQ ID NO: 78, or in other embodiments, at least 75%, 80%, 85%, 90%, or 95% identity with SEQ ID NO: 78. In some embodiments, the Klf4 protein comprises an amino acid sequence having from 1 to 20 amino acid insertions, deletions, or substitutions (collectively) with respect to SEQ ID NO: 78. Or in other embodiments, the Klf4 protein comprises an amino acid sequence having from 1 to 15 or from 1 to 10 amino acid insertions, deletions, or substitutions (collectively) with respect to SEQ ID NO: 78.

(SEQ ID NO: 78)
QPPGESDMAVSDALLPSFSTFASGPAGREKTLRQAGAPNNRWREELSHMK
RLPPVLPGRPYDLAAATVATDLESGGAGAACGGSNLAPLPRRETEEFNDL
LDLDFILSNSLTHPPESVAATVSSSASASSSSSPSSSGPASAPSTCSFTY
PIRAGNDPGVAPGGTGGGLLYGRESAPPPTAPFNLADINDVSPSGGFVAE
LLRPELDPVYIPPQQPQPPGGGLMGKFVLKASLSAPGSEYGSPSVISVSK
GSPDGSHPVVVAPYNGGPPRTCPKIKQEAVSSCTHLGAGPPLSNGHRPAA
HDFPLGRQLPSRTTPTLGLEEVLSSRDCHPALPLPPGFHPHPGPNYPSFL
PDQMQPQVPPLHYQELMPPGSCMPEEPKPKRGRRSWPRKRTATHTCDYAG
CGKTYTKSSHLKAHLRTHTGEKPYHCDWDGCGWKFARSDELTRHYRKHTG
HRPFQCQKCDRAFSRSDHLALHMKRHF

By “c-Myc protein” is meant a protein that is encoded by the MYC gene, or a natural or engineered variant, family-member, orthologue, fragment or fusion construct thereof, by way of non-limiting example, human c-Myc protein, mouse c-Myc protein, l-Myc protein, c-Myc (T58A) protein, a DNA-binding domain of c-Myc protein or a c-Myc-GFP fusion protein. In some embodiments the c-Myc protein comprises an amino acid sequence that has at least 70% identity with SEQ ID NO: 79, or in other embodiments, at least 75%, 80%, 85%, 90%, or 95% identity with SEQ ID NO: 79. In some embodiments, the c-Myc protein comprises an amino acid having from 1 to 20 amino acid insertions, deletions, or substitutions (collectively) with respect to SEQ ID NO: 79. Or in other embodiments, the c-Myc protein comprises an amino acid sequence having from 1 to 15 or from 1 to 10 amino acid insertions, deletions, or substitutions (collectively) with respect to SEQ ID NO: 79.

(SEQ ID NO: 79)
MDFFRVVENQQPPATMPLNVSFTNRNYDLDYDSVQPYFYCDEEENFYQQQ
QQSELQPPAPSEDIWKKFELLPTPPLSPSRRSGLCSPSYVAVTPFSLRGD
NDGGGGSFSTADQLEMVTELLGGDMVNQSFICDPDDETFIKNIIIQDCMW
SGFSAAAKLVSEKLASYQAARKDSGSPNPARGHSVCSTSSLYLQDLSAAA
SECIDPSVVFPYPLNDSSSPKSCASQDSSAFSPSSDSLLSSTESSPQGSP
EPLVLHEETPPTTSSDSEEEQEDEEEIDVVSVEKRQAPGKRSESGSPSAG
GHSKPPHSPLVLKRCHVSTHQHNYAAPPSTRKDYPAAKRVKLDSVRVLRQ
ISNNRKCTSPRSSDTEENVKRRTHNVLERQRRNELKRSFFALRDQIPELE
NNEKAPKVVILKKATAYILSVQAEEQKLISEEDLLRKRREQLKHKLEQLR
NSCA

Any aspect or embodiment described herein can be combined with any other aspect or embodiment as disclosed herein.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Gene Editing Proteins with Nickase Functionality Enabled Scarless Targeted Gene Insertion in Primary Human Cells

In this example, gene-editing proteins are disclosed, which were shown to provide surprisingly efficient and accurate single-stranded breaks that do not introduce unwanted mutations and have a reduced chance of off-target mutations.

In the example, a first gene-editing protein comprising a nuclease domain capable of binding to DNA and forming a dimer with another nuclease domain, and capable of creating a single-stranded break in a DNA site, was combined with a second gene-editing protein comprising a nuclease domain capable of binding to the DNA and forming a dimer with another nuclease domain, and incapable of creating a single-stranded break in a DNA site.

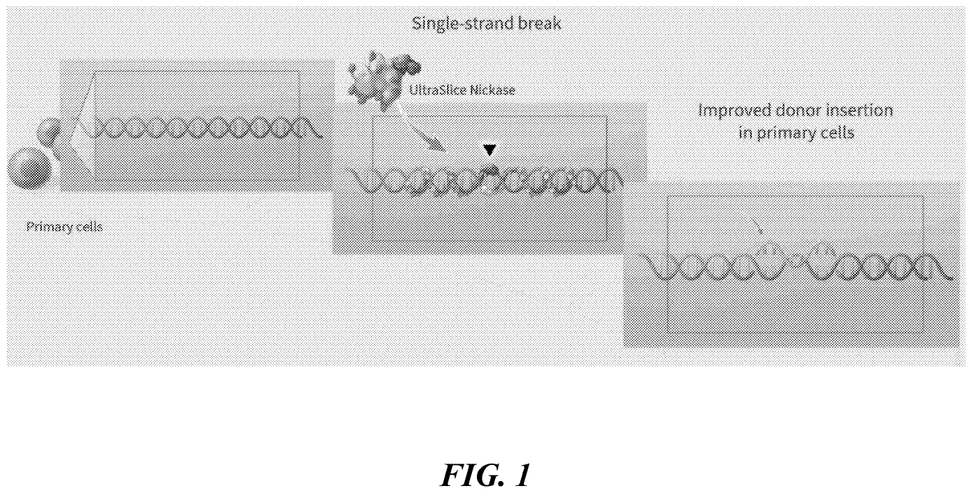

FIG. 1 is a schematic showing that gene editing proteins of the present disclosure, which comprise one mutant nuclease domain, can enable scarless targeted gene insertion in primary human cells.

To reduce non-homologous end joining (NHEJ) or microhomology-mediated end joining (MMEJ) activity, nickases were designed which rendered the FokI nuclease domain inactive through targeted point mutations. These mutations to the FokI nuclease domain retained their DNA binding ability and dimerization of the two gene editing proteins in a pair. However, the FokI domain mutation prevented cleavage of both strands of the DNA, and instead, produced a single stranded break (SSB) in the DNA. Mutant COL7A1_e73 gene editing proteins were created which target and bind to exon 73 of the COL7A1. Exon 73 was selected since certain defects in this exon are known to cause dystrophic epidermolysis bullosa. The ability of mutant COL7A1_e73 gene editing proteins, with a FokI domain mutant in one gene editing protein of a pair and a wild-type FokI domain in the other gene editing protein of the pair, were tested when for their non-homologous end joining (NHEJ) or microhomology-mediated end joining (MMEJ) activity and insertion efficiency. Specifically, the mutated gene editing proteins were tested for their ability cleave DNA of exon 73 in COL7A1. The mutated gene editing proteins tested included combinations of three mutations previously reported to confer nickase functionality to FokI, a Type IIS restriction endonuclease. Each mutation was a single base substitution that results in a single amino acid change; for D450A and D467A, the change was from Asp to Ala, whereas for D450N, the change was from Asp to Asn.

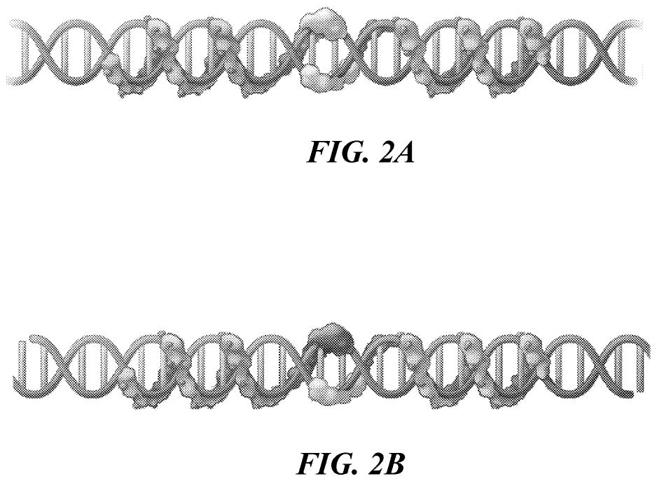

FIG. 2A is an illustration of a wild type COL7A1_e73 gene-editing protein pair bound to target DNA. FIG. 2B is an illustration of COL7A1_e73 gene editing protein of the present disclosure, which comprises one mutant FokI domain and is bound to target DNA. Here, the mutated FokI domain is in darker shade.

Table 1 below provides explanation of nomenclature and point mutations to the FokI domain sequence as used in this Example. The pair US22 consists of a pair of wild-type gene-editing proteins (US2L and US2R). Underlining in Table 1 represents the location of the respective point mutation in each US2R right gene-editing protein mutant (D450A, D450N, and D467A). The letters in bold font in Table 1 represent either the wild-type base in the normal right gene-editing protein (US2R) or the mutated base in said right gene-editing protein which resulted in the US2R variants D450A, D450N, and D467A. Table 2 provides a summary of these constructs in relation to FokI and Table 3 provides a summary of nucleic acid sequences encoding these constructs.

TABLE 1
FokI Domain Mutations

| Pair | Left | Right | Right Protein |
|---|---|---|---|
| Name | Protein | Protein | DNA Sequence |
| US22 | US2L | US2R | AAACCGGACGGAGCAATT |
| | | | 450 |
| | | | ATCGTGGCTACTAAGCTT |
| | | | 467 |
| USN1 | US2L | US2R | AAACCGGCCGGAGGAATT |
| | | (D450A) | 450 |
| USN2 | US2L | US2R | AAACCGAACGGAGGAATT |
| | | (D450N) | 450 |
| USN3 | US2L | US2R | ATCGTGGATACTAAGCTT |
| | | (D467A) | 467 |

TABLE 2
FokI Mutant Proteins

| Name | SEQ ID NO: | Sequence |
|---|---|---|
| FokI-Wild | 80 | QLVKSELEEKKSELRHKLKYVPHEYIELIEIARNSTQDRIL |
| Type | | EMKVMEFFMKVYGYRGKHLGGSRKPDGAIYTVGSPIDY |
| | | GVIVDTKAYSGGYNLPIGQADEMQRYVEENQTRNKHIN |
| | | PNEWWKVYPSSVTEFKFLFVSGHFKGNYKAQLTRLNHI |
| | | TNCNGAVLSVEELLIGGEMIKAGTLTLEEVRRKFNNGEI |
| | | NFRS |
| FokI- | 81 | QLVKSELEEKKSELRHKLKYVPHEYIELIEIARNSTQDRIL |
| D67A | | EMKVMEFFMKVYGYRGKHLGGSRKPAGAIYTVGSPIDY |
| | | GVIVDTKAYSGGYNLPIGQADEMQRYVEENQTRNKHIN |
| | | PNEWWKVYPSSVTEFKFLFVSGHFKGNYKAQLTRLNHI |
| | | TNCNGAVLSVEELLIGGEMIKAGTLTLEEVRRKFNNGEI |
| | | NFRS |
| FokI- | 82 | QLVKSELEEKKSELRHKLKYVPHEYIELIEIARNSTQDRIL |
| D67N | | EMKVMEFFMKVYGYRGKHLGGSRKPNGAIYTVGSPIDY |
| | | GVIVDTKAYSGGYNLPIGQADEMQRYVEENQTRNKHIN |
| | | PNEWWKVYPSSVTEFKFLFVSGHFKGNYKAQLTRLNHI |
| | | TNCNGAVLSVEELLIGGEMIKAGTLTLEEVRRKENNGEI |
| | | NFRS |
| FokI- | 83 | QLVKSELEEKKSELRHKLKYVPHEYIELIEIARNSTQDRIL |
| D84A | | EMKVMEFFMKVYGYRGKHLGGSRKPDGAIYTVGSPIDY |
| | | GVIVATKAYSGGYNLPIGQADEMQRYVEENQTRNKHIN |
| | | PNEWWKVYPSSVTEFKFLFVSGHFKGNYKAQLTRLNHI |
| | | TNCNGAVLSVEELLIGGEMIKAGTLTLEEVRRKENNGEI |
| | | NFRS |

TABLE 3
Nucleic Acid Sequences Encoding FokI Mutant Proteins

| Name | SEQ ID NO: | Sequence |
|---|---|---|
| FokI-Wild | 84 | CAACTCGTGAAGAGTGAACTTGAGGAGAAAAAGTCG |
| Type | | GAGCTGCGGCACAAATTGAAATACGTACCGCATGAAT |
| | | ACATCGAACTTATCGAAATTGCTAGGAACTCGACTCA |
| | | AGACAGAATCCTTGAGATGAAGGTAATGGAGTTCTTT |
| | | ATGAAGGTTTATGGATACCGAGGGAAGCATCTCGGTG |
| | | GATCACGAAAACCCGACGGAGCAATCTATACGGTGGG |
| | | GAGCCCGATTGATTACGGAGTGATCGTCGACACGAAA |
| | | GCCTACAGCGGTGGGTACAATCTTCCCATCGGGCAGG |
| | | CAGATGAGATGCAACGTTATGTCGAAGAAAATCAGAC |
| | | CAGGAACAAACACATCAATCCAAATGAGTGGTGGAA |
| | | AGTGTATCCTTCATCAGTGACCGAGTTTAAGTTTTTGT |
| | | TTGTCTCTGGGCATTTCAAAGGCAACTATAAGGCCCA |
| | | GCTCACACGGTTGAATCACATTACGAACTGCAATGGT |
| | | GCGGTTTTGTCCGTAGAGGAACTGCTCATTGGTGGAG |
| | | AAATGATCAAAGCGGGAACTCTGACACTGGAAGAAG |
| | | TCAGACGCAAGTTTAACAATGGCGAGATCAATTTCCG |
| | | CTCATAA |
| FokI- | 85 | CAACTCGTGAAGAGTGAACTTGAGGAGAAAAAGTCG |
| D67A | | GAGCTGCGGCACAAATTGAAATACGTACCGCATGAAT |
| | | ACATCGAACTTATCGAAATTGCTAGGAACTCGACTCA |
| | | AGACAGAATCCTTGAGATGAAGGTAATGGAGTTCTTT |
| | | ATGAAGGTTTATGGATACCGAGGGAAGCATCTCGGTG |
| | | GATCACGAAAACCCgccGGAGCAATCTATACGGTGGGG |
| | | AGCCCGATTGATTACGGAGTGATCGTCGACACGAAAG |
| | | CCTACAGCGGTGGGTACAATCTTCCCATCGGGCAGGC |
| | | AGATGAGATGCAACGTTATGTCGAAGAAAATCAGACC |
| | | AGGAACAAACACATCAATCCAAATGAGTGGTGGAAA |
| | | GTGTATCCTTCATCAGTGACCGAGTTTAAGTTTTTGTT |
| | | TGTCTCTGGGCATTTCAAAGGCAACTATAAGGCCCAG |
| | | CTCACACGGTTGAATCACATTACGAACTGCAATGGTG |
| | | CGGTTTTGTCCGTAGAGGAACTGCTCATTGGTGGAGA |
| | | AATGATCAAAGCGGGAACTCTGACACTGGAAGAAGTC |
| | | AGACGCAAGTTTAACAATGGCGAGATCAATTTCCGCT |
| | | CATAA |
| FokI- | 86 | CAACTCGTGAAGAGTGAACTTGAGGAGAAAAAGTCG |
| D67N | | GAGCTGCGGCACAAATTGAAATACGTACCGCATGAAT |
| | | ACATCGAACTTATCGAAATTGCTAGGAACTCGACTCA |
| | | AGACAGAATCCTTGAGATGAAGGTAATGGAGTTCTTT |
| | | ATGAAGGTTTATGGATACCGAGGGAAGCATCTCGGTG |
| | | GATCACGAAAACCCaacGGAGCAATCTATACGGTGGGG |
| | | AGCCCGATTGATTACGGAGTGATCGTCGACACGAAAG |
| | | CCTACAGCGGTGGGTACAATCTTCCCATCGGGCAGGC |
| | | AGATGAGATGCAACGTTATGTCGAAGAAAATCAGACC |
| | | AGGAACAAACACATCAATCCAAATGAGTGGTGGAAA |
| | | GTGTATCCTTCATCAGTGACCGAGTTTAAGTTTTTGTT |
| | | TGTCTCTGGGCATTTCAAAGGCAACTATAAGGCCCAG |
| | | CTCACACGGTTGAATCACATTACGAACTGCAATGGTG |
| | | CGGTTTTGTCCGTAGAGGAACTGCTCATTGGTGGAGA |
| | | AATGATCAAAGCGGGAACTCTGACACTGGAAGAAGTC |
| | | AGACGCAAGTTTAACAATGGCGAGATCAATTTCCGCT |
| | | CATAA |
| FokI- | 87 | CAACTCGTGAAGAGTGAACTTGAGGAGAAAAAGTCG |
| D84A | | GAGCTGCGGCACAAATTGAAATACGTACCGCATGAAT |
| | | ACATCGAACTTATCGAAATTGCTAGGAACTCGACTCA |
| | | AGACAGAATCCTTGAGATGAAGGTAATGGAGTTCTTT |
| | | ATGAAGGTTTATGGATACCGAGGGAAGCATCTCGGTG |
| | | GATCACGAAAACCCGACGGAGCAATCTATACGGTGGG |
| | | GAGCCCGATTGATTACGGAGTGATCGTCgccACGAAAG |
| | | CCTACAGCGGTGGGTACAATCTTCCCATCGGGCAGGC |
| | | AGATGAGATGCAACGTTATGTCGAAGAAAATCAGACC |
| | | AGGAACAAACACATCAATCCAAATGAGTGGTGGAAA |
| | | GTGTATCCTTCATCAGTGACCGAGTTTAAGTTTTTGTT |
| | | TGTCTCTGGGCATTTCAAAGGCAACTATAAGGCCCAG |
| | | CTCACACGGTTGAATCACATTACGAACTGCAATGGTG |
| | | CGGTTTTGTCCGTAGAGGAACTGCTCATTGGTGGAGA |
| | | AATGATCAAAGCGGGAACTCTGACACTGGAAGAAGTC |
| | | AGACGCAAGTTTAACAATGGCGAGATCAATTTCCGCT |
| | | CATAA |

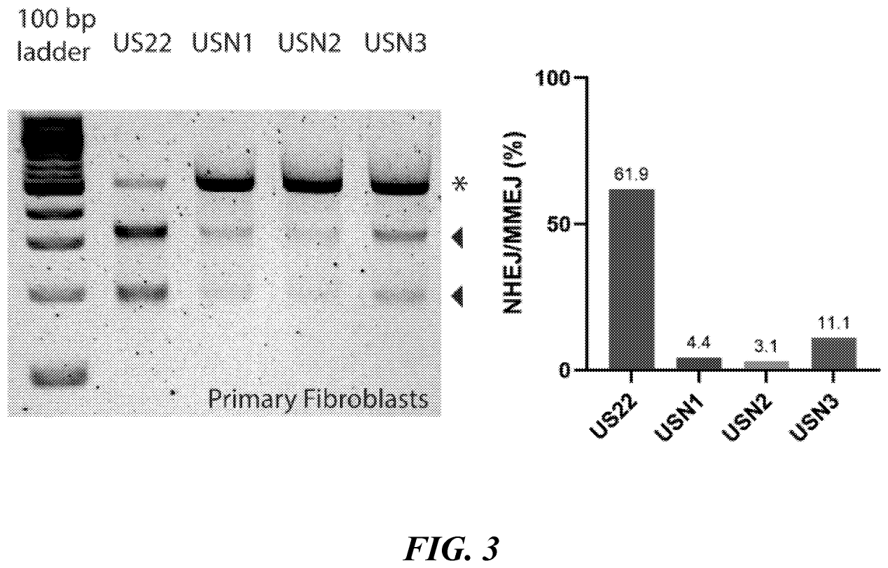

FIG. 3 shows primary human fibroblast electroporation results comparing the NHEJ or MMEJ activity of all four gene editing protein pairs. FIG. 3 shows on the left a 2% agarose gel T7E1 assay with an asterisk indicating wildtype bands and arrows pointing to cut bands below as evidence of NHEJ or MMEJ activity. FIG. 3 shows on the right a graph quantifying relative intensity of cut bands, showing reduction of NHEJ/MMEJ in all gene editing protein pair samples with the mutant USN2 exhibiting the lowest level. The percentage of NHEJ or MMEJ activity observed with each pair of gene proteins is as follows: for wild-type (US22), 61.9%; for a D450A mutant (USN1), 4.4%; for a D450N mutant (USN2), 3.1%, and for a D467A mutant (USN3), 11.1%. This demonstrates that mutant COL7A1_e73 gene editing proteins comprising the D450A mutation or D450N mutations resulted in significantly reduced NHEJ relative to the COL7A1_e73 gene-editing protein pair with a both wild-type FokI cleavage domain. Notably, the D450N mutant exhibited the least amount of NHEJ. In other words, by using an illustrative mutant gene-editing protein of the present disclosure, which include one mutant FokI nuclease domain, NHEJ/MMEJ was reduced from about 6- to about 20-fold.

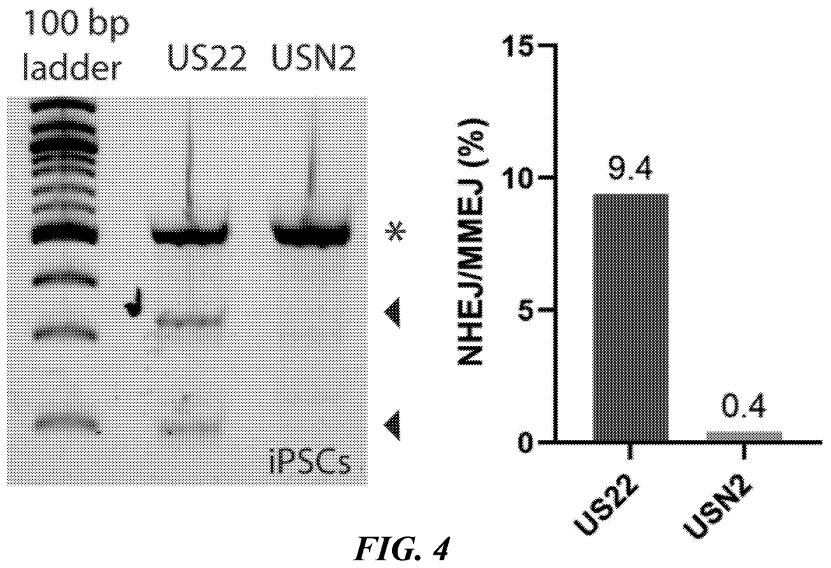

FIG. 4 shows induced pluripotent stem cell (iPSC) electroporation results comparing the NHEJ or MMEJ activity of the wild-type US22 editing protein pair and the mutant USN2 gene editing protein pair. FIG. 4 shows one the left a 2% agarose gel T7E1 assay with an asterisk pointing to wildtype bands and arrows pointing to cut bands below as a as evidence of NHEJ or MMEJ activity. FIG. 4 shows on the right a graph quantifying the relative intensity of cut bands, showing a reduction of NHEJ/MMEJ in USN2, which exhibited an incidence of NHEJ or MMEJ of 0.4% whereas US22 exhibited an incidence of NHEJ or MMEJ of 9.4%. In other words, by using the gene-editing proteins of the present disclosure, which include one mutant nuclease domain, NHEJ/MMEJ was reduced from about 23-fold.

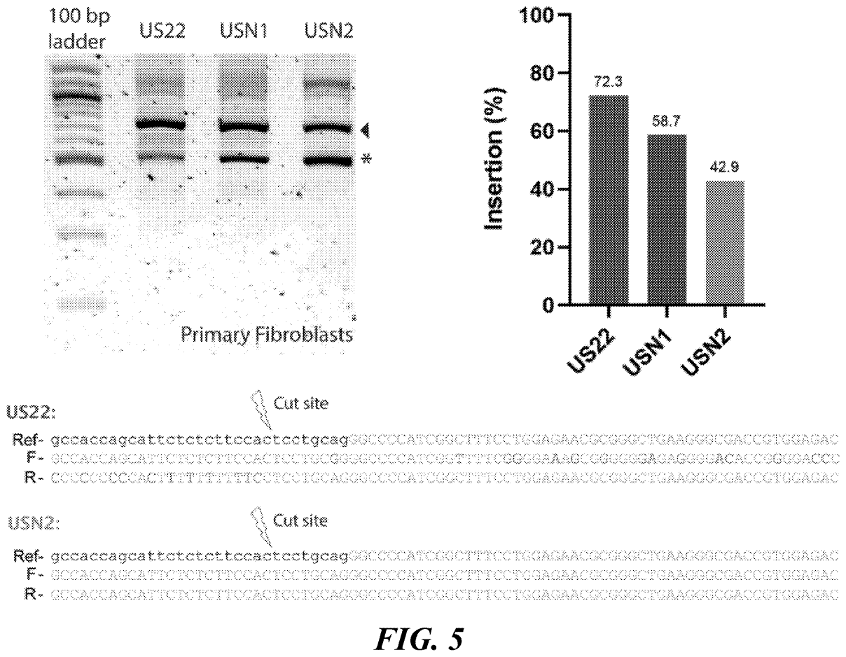

FIG. 5 shows the relative insertion efficiency of gene-editing protein pairs of a 300-base pair (bp) double stranded DNA (dsDNA) donor sequence into primary human fibroblasts following electroporation. FIG. 5 presents a 2% agarose gel (top left) showing PCR-amplified genomic DNA (gDNA) with an arrow pointing to insertion bands and asterisk indicating wildtype bands below. FIG. 5 shows a graph (top right) illustrating quantified relative insertion levels. The wild-type US22 exhibited a relative insertion efficiency of 72.3%. whereas the mutants had relative insertion efficiencies of 58.7% (USN1) and 42.9% (USN2). FIG. 5 shows at the bottom Sanger sequencing of wildtype bands (535 bp) from US22 and USN2 fibroblast electroporation. Red letters indicate NHEJ/MMEJ. Sanger Sequencing comparing the D450N-treated PCR amplicon to a wild-type COL7A1 PCR amplicon confirmed gene-editing efficiency and indicated no significant alteration of the genomic target site by D450N as shown in FIG. 5. The data shown in FIG. 5 further demonstrates the ability of said gene-editing proteins to insert a 300 bp dsDNA donor sequence via electroporation into primary human fibroblasts. Insertion band intensities show high insertion efficiencies for D450A and D450N (58.7% and 42.9%, respectively), though lower than the US22 gene-editing protein pair (72.3%). These data show that the mutant gene-editing proteins are capable of facilitating site-specific insertion of repair templates.

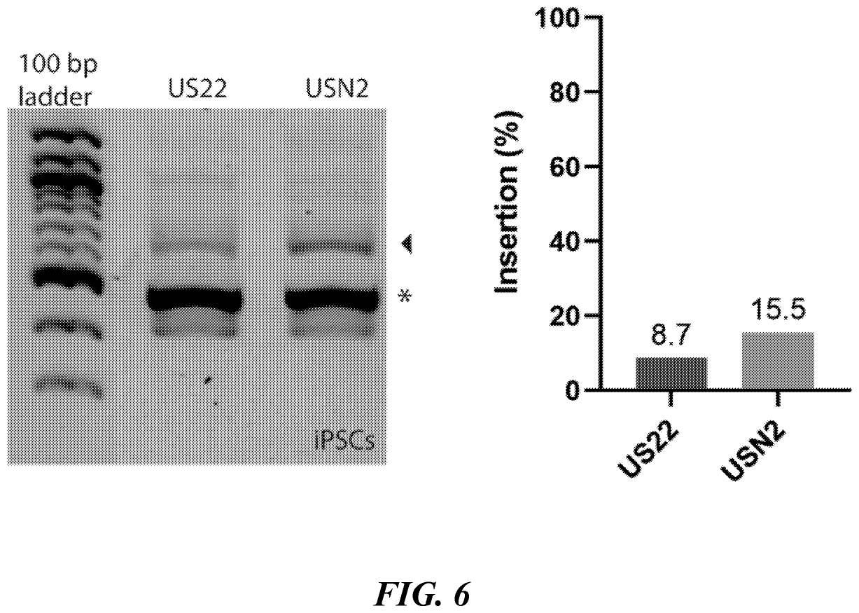

FIG. 6 shows the relative insertion efficiency of gene-editing protein pairs of a 300 bp dsDNA donor sequence into human iPSCs following electroporation. FIG. 6 shows a 2% agarose gel (left) showing PCR-amplified gDNA with an arrow pointing to insertion bands and asterisk indicating wildtype bands below. FIG. 6 shows on the right a graph illustrating quantified relative insertion levels where the mutant gene-editing protein USN2 provides greater insertion (15.5%) than the wild-type US22 (8.7%). The overall reduction in insertion is due to the use of dsDNA when ssDNA would have been optimal.

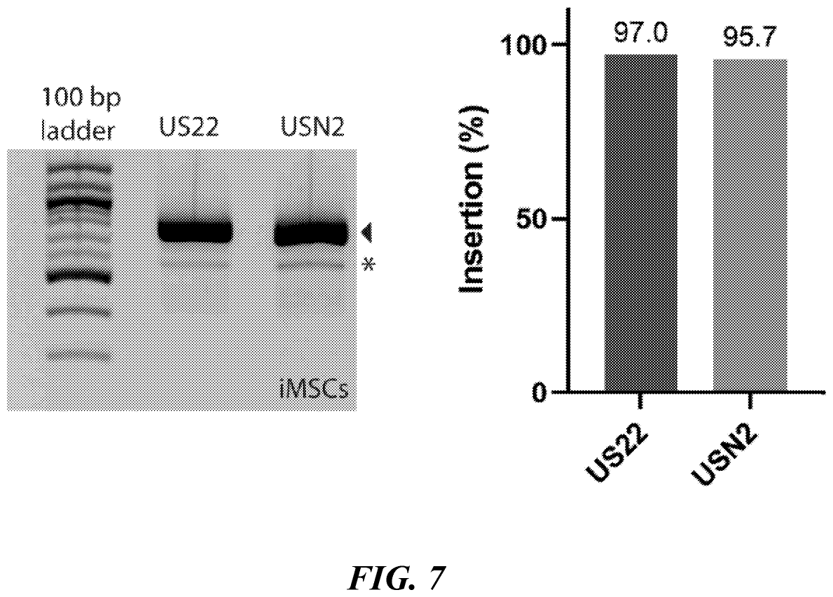

Finally, FIG. 7 shows the relative insertion efficiency of gene-editing protein pairs of a 300 nucleotide (nt) single stranded DNA (ssDNA) donor sequence into human iMSCs following electroporation. FIG. 7 presents on the left a 2% agarose gel showing PCR-amplified gDNA with an arrow pointing to insertion bands and asterisk indicating wildtype bands below. FIG. 7 presents on the right a graph illustrating quantified relative insertion levels which were nearly identical: with the wild-type protein (US22) having 97.0% relative levels of insertion and the mutant protein (USN2) providing 95.7% relative levels of insertion.

In summary, the data presented in this example demonstrate that the gene-editing protein pairs disclosed herein and which comprise a mutation in one nuclease domain enable scarless insertion of donor DNA into defined genomic loci in both primary human cells and human stem cell lines. Said gene-editing protein pairs have the potential to improve the safety of in vivo gene insertion by reducing off-target effects. Furthermore, gene editing by the gene-editing protein pairs disclosed herein may increase the efficiency of in vivo gene insertion by allowing for repeat dosing.

### Example 2: Efficient Transgene Knock-In in Human iPS Cells Combined with Small Molecule-Mediated “On-Switch” Yields Clonal Populations of Engineered Tissue-Specific Cells

This example demonstrates methods for providing control over expression of genes that are normally turned off during specific periods of differentiation. Currently, when skilled artisans want to activate genes during cell culturing, typically the artisans add components (e.g., cytokines) to a culturing medium. These tools can be considered to be “external to the cell”. In contrast, here, control relies on promoters which have temporal/stage specific regulation. By harnessing these promoters along with small molecule compounds, e.g., those that demethylate DNA, a promoter can be reactivated, and genes can be turned on at specific times during a cell's differentiation.

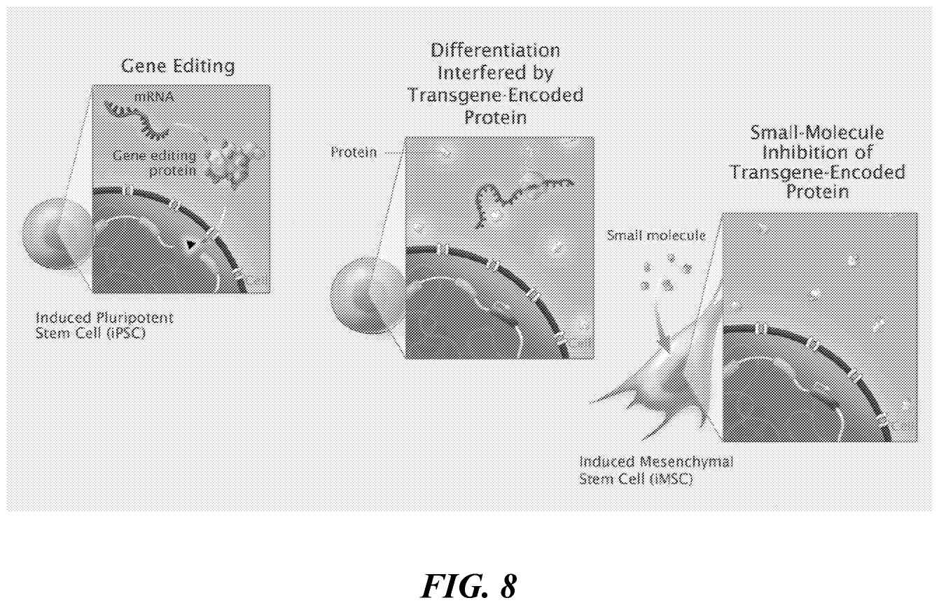

FIG. 8 is a schematic showing that efficient transgene knock-in in human induced pluripotent stem cells (hiPSCs) combined with small molecule treatment yields clonal populations of engineered tissue-specific cells.

In this Example, stable clonal hiPS cell lines were established and engineered to host a GFP-expressing cassette in the AAVS safe-harbor locus using two different promoters and then the engineered hiPSCs were differentiated to engineered iPSC-derived mesenchymal stem cells (EiMSCs) while changes in GFP expression were monitored.

### Methodology

1. Transfect hiPSCs with a chromatin context-sensitive gene editing endonuclease according to some embodiments of the present disclosure, mRNA and single stranded DNA repair templates encoding GFP sequence under JeT (“pJet”) and Ef1a promoters.
2. Perform gDNA extraction and AAVS1 surveyor PCR to confirm ssDNA repair template integration at AAVS1 locus
3. Perform single cell sorting to obtain homogeneous cell line started from a single cell
4. Using Stemdiff™ Mesenchymal Progenitor kit, differentiate iPS cell lines inserted with pJet and Ef1a GFP sequence in their genomes into iMSCs.
5. Verify robust iMSC differentiation using surface marker expression at the completion of differentiation.

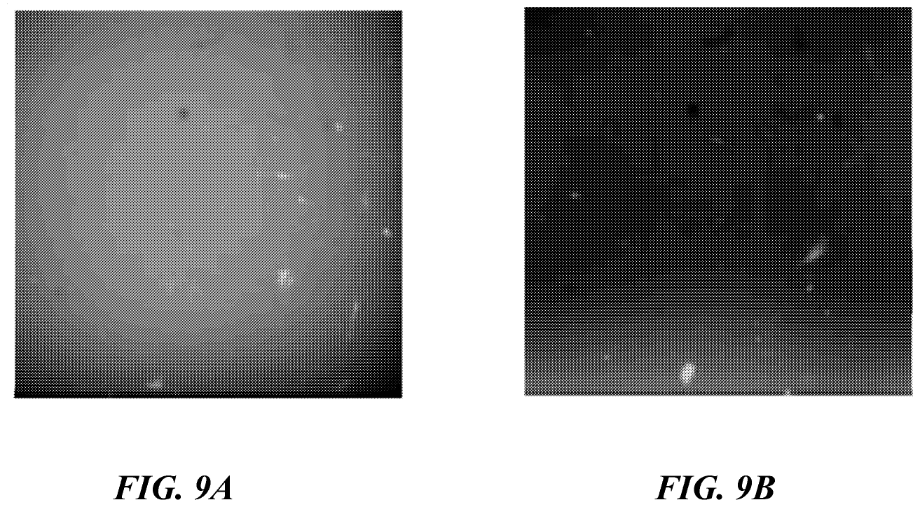

FIG. 9 shows direct integration of a GFP sequence under pJet and Ef1a promoters into iMSCs. FIG. 9A shows GFP is expressed under a pJet promoter when directly inserted to an AAVS1 site in iMSCs. FIG. 9B shows GFP expressed under an Ef1a promoter when directly inserted to a AAVS1 site in iMSCs.

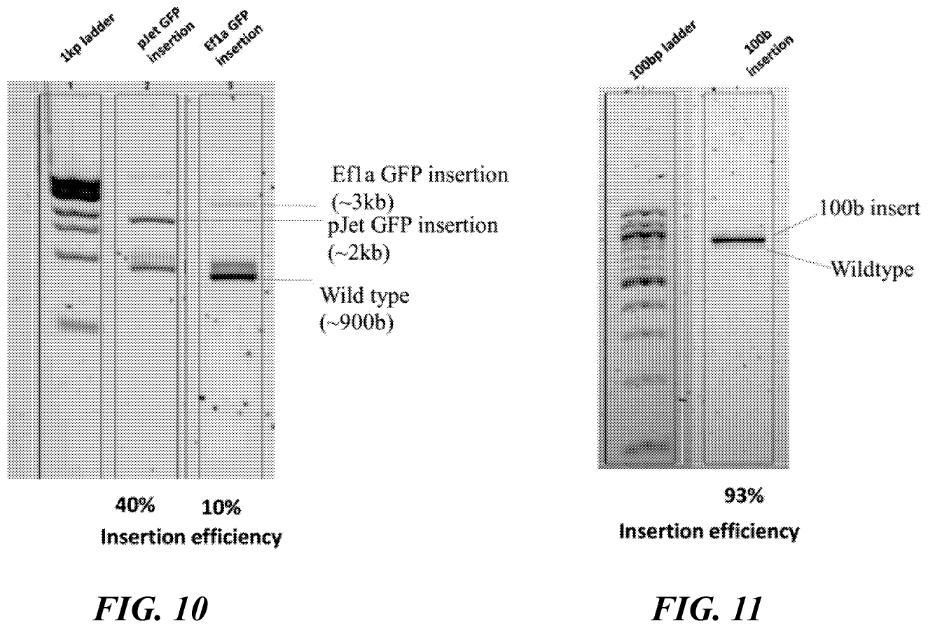

FIG. 10 is a gel image showing AAVS1 surveyor PCR amplicon with a wild-type DNA band at around 900b and amplicons with pJet and Ef1a GFP sequence insertion each at around 2 kb and 3.2 kb, respectively. The efficiency of pJet and Ef1a GFP sequence insertion into an AAVS1 site was 40% and 10%, respectively.

FIG. 11 shows that the gene-editing proteins enabled high efficiency integration of ssDNA repair templates into an AAVS1 site in iPSCs. Co-transfection of gene-editing proteins with mRNA and ssDNA repair templates encoding randomized 100b sequence showed 93% insertion efficiency.

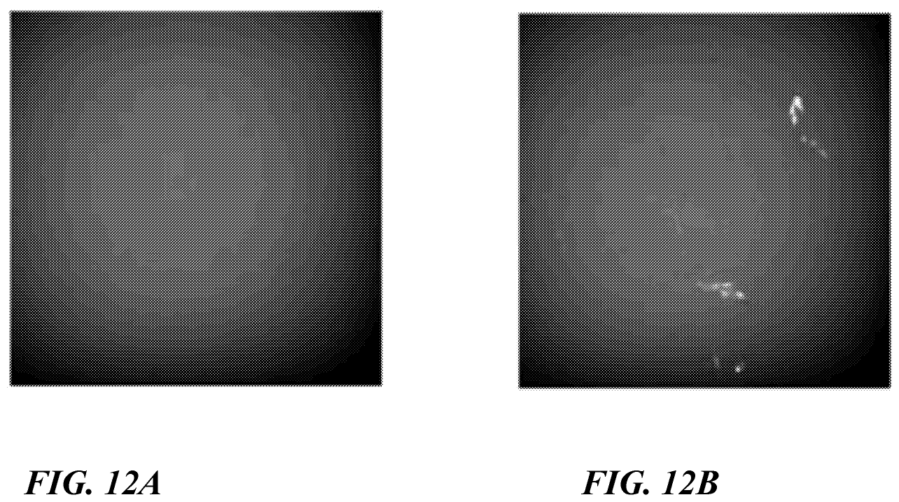

FIG. 12 shows direct insertion of GFP under either a pJet or Ef1a promoter into an AAVS1 locus in hiPSCs. While EF1α and JeT promoters drove robust GFP expression when inserted directly in iMSCs, in iPS cells strong GFP expression was observed under EF1α whereas expression, but was not detected in JeT-GFP iPS cells. FIG. 12A shows that GFP was not expressed under a pJet promoter when directly inserted to an AAVS1 site in hiPSCs. FIG. 12B shows that GFP was expressed under an Ef1a promoter when directly inserted to an AAVS1 site in hiPSCs.

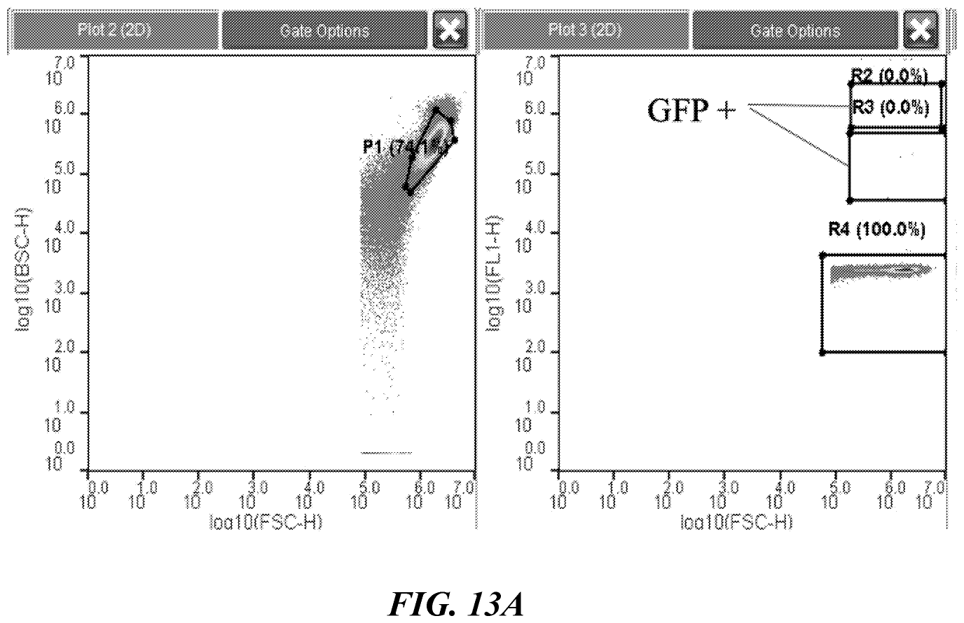

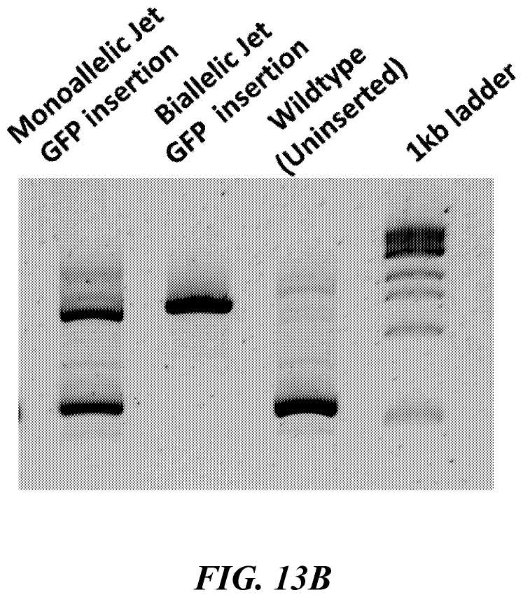

FIGS. 13A-13B show cell line development of a JeT-GFP sequence inserted into iPSCs. FIG. 13A shows GFP under a pJet promoter was not expressed in iPSCs, such that single cells were sorted based on forward and backward scatter. FIG. 13B shows a gel showing monoallelic, biallelic, and uninserted amplicons. The biallelic inserted population was selected for cell line expansion.

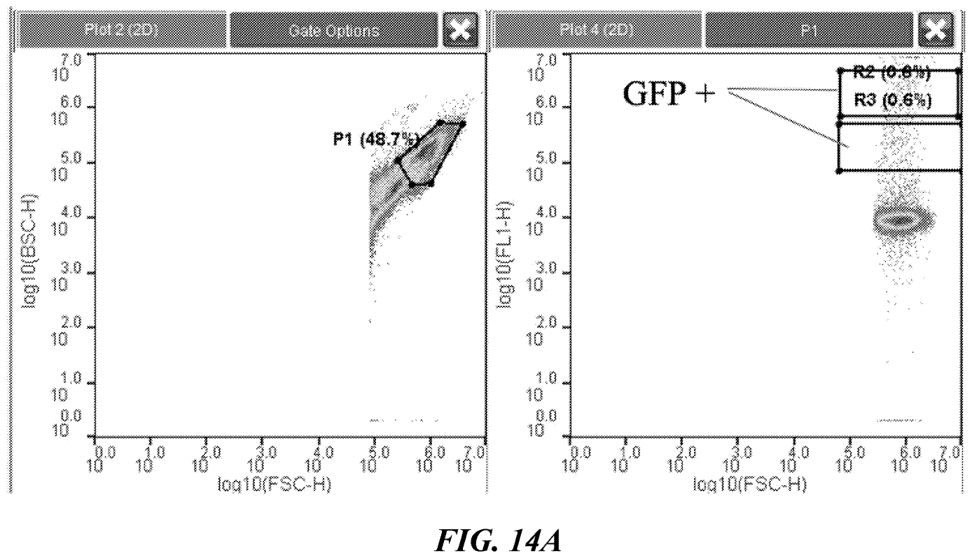

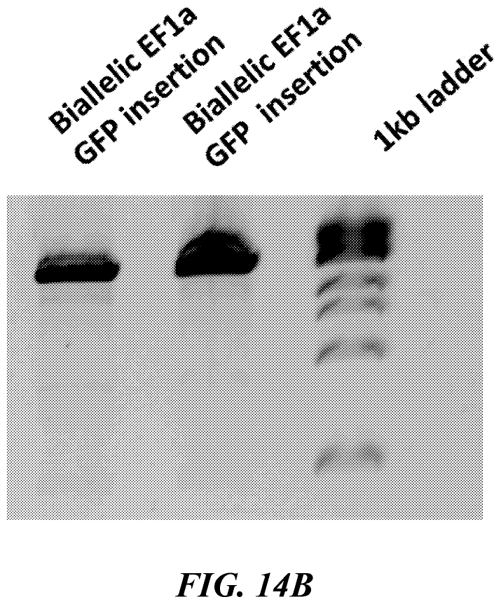

FIGS. 14A-14B show cell line development of an Ef1a GFP sequence inserted iPSCs. FIG. 14A shows GFP expressing cells that were sorted for cell line development. FIG. 14B shows a picture of a gel showing biallelic insertion of an Ef1a GFP sequence into an AAVS1 locus which was selected for cell line expansion.

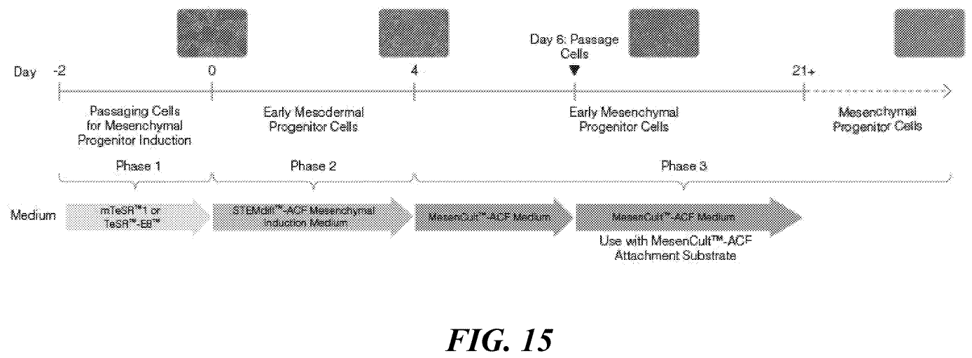

FIG. 15 shows a flowchart of the Stemdiff™ Mesenchymal Progenitor kit protocol for differentiating iPSCs to iMSCs.

Engineered iPS cells were differentiated into EiMSCs, and GFP expression was monitored.

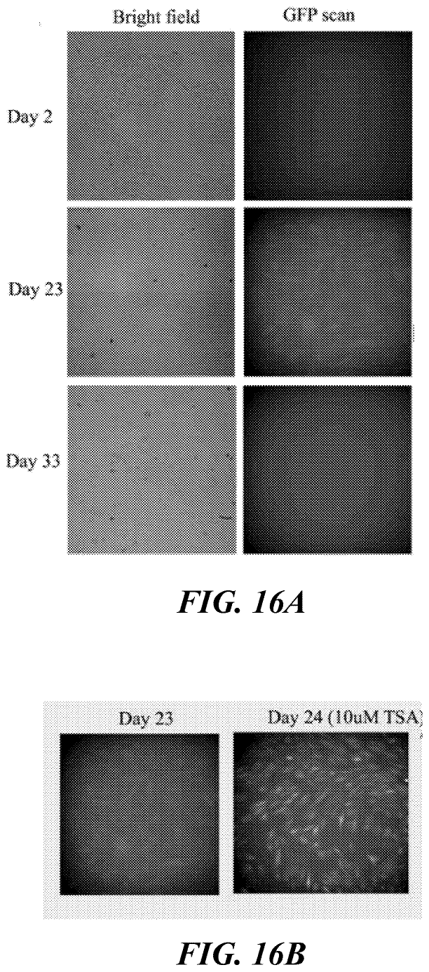

FIG. 16A and FIG. 16B show the tracking of GFP expression under a pJet promoter during iPSC to iMSC differentiation. FIG. 16A shows bright field and GFP images that were taken from day 2 to day 33 at which the differentiation is completed. GFP was expressed from day 18 to 30 but was completely silenced on day 33. FIG. 16B shows that, to reactivate the pJet promoter, 10 μM TSA (Trichostatin A) differentiating cells were treated for 24 hours on day 23. The image shows that there was a noticeable increase of GFP expression post TSA treatment, indicating that TSA treatment reactivated, i.e., was an “on switch”, to a silenced pJet promoter.

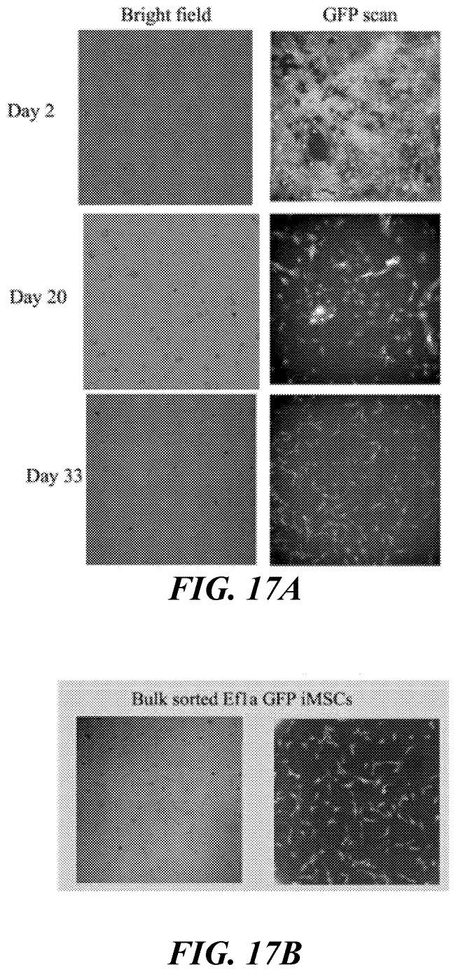

FIG. 17 shows the tracking of GFP expression under an Ef1a promoter during iPSC to iMSC differentiation. FIG. 17A shows bright field and GFP images that were taken from day 2 to day 33 at which the differentiation is completed. GFP expression was robust until day 20, but a loss of GFP expressing cells was observed. By day 33, only ˜30% of cells seemed to express GFP. FIG. 17B shows that in order to enrich a GFP expressing population, bulk sorting was done of GFP positive iMSCs. Approximately 95% of iMSCs express GFP post bulk sorting for more than 5 weeks. In other words, the reporter gene GFP driven by the Ef1a promoter continues to be expressed in the differentiated iMSCs, but to a lesser extent.

To obtain a clonal population of EiMSCs that uniformly express GFP, GFP-expressing EF1α-GFP iMSCs were enriched, resulting in a cell line that exhibited traditional MSC surface markers (positive markers: CD90, CD73, CD105, and CD44; negative markers:CD34, TRA-1-60, TRA-1-81, CD45, and HLA-DR) and displayed stable GFP expression for over seven passages.

Table 4 below shows iMSC verification by surface marker expression. Flow cytometry analysis showed that differentiated Ef1a GFP iMSCs exhibited surface marker expression pattern similar to wildtype iMSCs, indicating robust differentiation of transgene-inserted hiPSCs to EiMSCs.

TABLE 4
MSC Marker Expression

| | Ef1a GFP iMSC | WT iMSC |
|---|---|---|
| CD90 | + (95%) | + (96%) |
| CD73 | + (85%) | + (96%) |
| CD44 | + (99%) | + (98%) |
| CD105 | + (87%) | + (97%) |
| CD34 | − (23%) | −(28%) |
| CD45 | − (12%) | − (50%) |
| TRA1-81 | − (0.9%) | − (2%) |
| TRA1-60 | − (10 | − (30%) |
| HLA-DR | − (1.4%) | − (0.6%) |

Taken together, these data demonstrate expression of the reporter gene (GFP) is driven by the JeT promoter or by EF1α promoter. Notably, the EF1α promoter drove expression of the GFP in iPSC but to a lesser extent in EiMSCs; the JeT promoter drove expression of the GFP as iPSCs differentiate but did not drive expression near the end of differentiation. During differentiation, the number of GFP-expressing cells decreased from >99% in the starting EF1α-GFP iPS cells to 40% in the differentiated EF1α-GFP iMSCs. In contrast, JeT-GFP iPS cells began expressing GFP during differentiation but stopped expressing GFP near the end of the differentiation process. Since the JeT promoter drove expression of the reporter gene as iPSCs differentiate but did not drive expression near the end of differentiation, to continue expression of the JeT-driven reporter gene, the cell was contacted with the small molecule Trichostatin A. Treatment with Trichostatin A, a selective histone deacetylase (HDAC) inhibitor which demethylates DNA, resulted in a temporary increase in GFP expression in JeT-GFP cells during differentiation.

This demonstrates that stable clonal hiPS cell lines were established and engineered to host a GFP-expressing cassette in the AAVS safe-harbor locus using two different promoters, and the engineered hiPS cell lines were differentiated into EiMSCs. During differentiation, by selecting specific promoters to drive expression of gene(s) of interest in combination with small molecule “on switches”, gene regulation can be controlled throughout differentiation of a cell.

The data disclosed herein demonstrate a platform for developing clonal EiMSC cell populations that uniformly and stably express a desired protein from a transgene inserted into a defined genomic locus by gene editing via transfected mRNA. And, importantly, the data also shows temporal control of transgene expression using small molecules during directed differentiation of iPS cells. This platform benefits from high knock-in efficiency enabled by mRNA gene editing combined with ssDNA donors.

In summary, the data presented in this Example indicate that GFP expression under an Ef1a promoter is robust in both iPSCs and differentiated iMSCs. Bulk sorted Ef1a GFP iMSCs maintained their GFP expression for >5 weeks. GFP expression under a pJet promoter appeared to be susceptible to silencing. TSA treatment successfully demethylated and reactivated the pJet promoter.

The data presented in this Example also demonstrate the feasibility of clonal EiMSC generation through hiPSCs engineered for transgene expression. The ability to generate an engineered clonal MSC line in a period of just three months is a breakthrough that may help allogeneic MSC therapies become commonplace in the future.

### Example 3: Chemically Modified Single-Stranded DNA Donors Enable Efficient mRNA Gene Editing-Mediated Knock-In in Human iPS Cells

This example discloses data demonstrating a method for high-yield synthesis of single-stranded DNA, which is suitable for cellular applications, including gene editing.

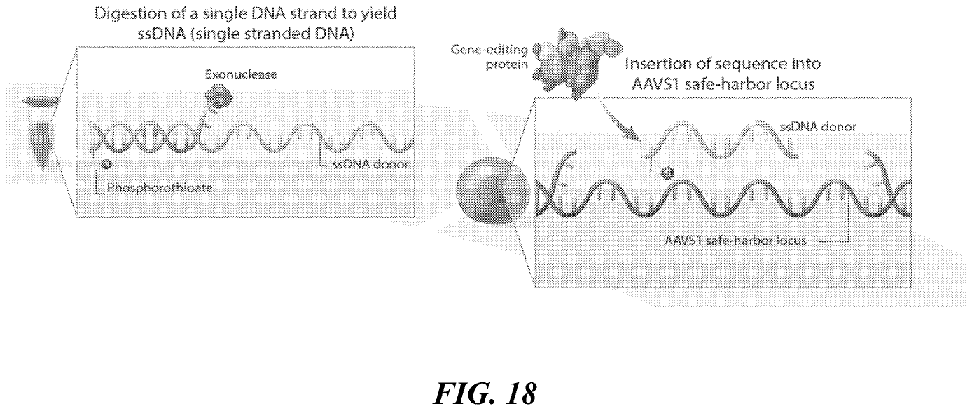

FIG. 18 is a cartoon showing that specific combinations of nucleases and modified nucleotides can create single-stranded DNA which can serve as a repair template during gene-editing protein. This single-stranded DNA is inserted into a single-strand breaks at specific genomic sites created by a gene-editing protein, which allows for integration of the single-stranded DNA to create a knock-in of a defined sequence.

Current methods of synthesizing single-stranded donor DNA—which exhibits lower toxicity and is less prone to random genomic integration than double-stranded DNA—for gene knock-in suffer from low yields, contamination with residual dsDNA, length limitations, and high cost. The data disclosed in this Example demonstrate an enzymatic approach for producing long (>2.8 kb) and concentrated (>1 μg/μL) ssDNA suitable for generation of knock-in lines of human induced pluripotent stem (hiPS) cells.

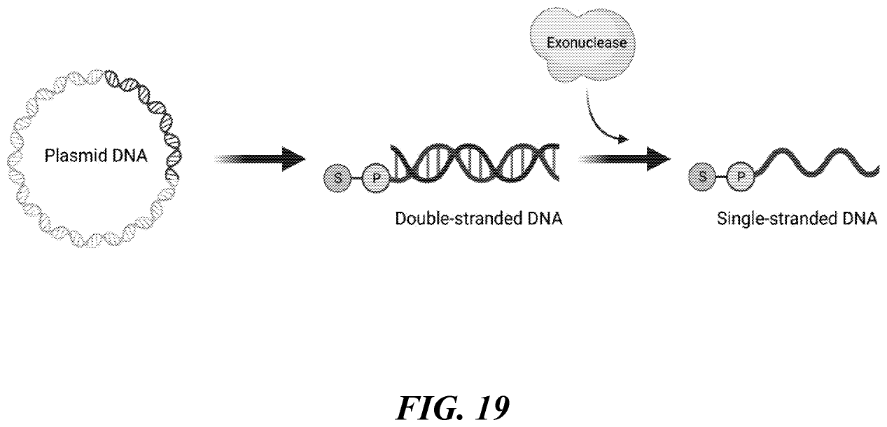

FIG. 19 is a cartoon showing an overview of single-stranded DNA synthesis from plasmid DNA. Double-stranded DNA was PCR amplified from plasmid templates using a modified forward primer that incorporated either one (“1-PS”) 5′-phosphorothioate-containing nucleotides or five consecutive 5′-phosphorothioate (“5-PS”)-containing nucleotides on the protein-encoding strand to protect against enzymatic digestion. The resulting PCR products were treated with exonucleases to eliminate the unprotected noncoding strand, thereby, generating single-stranded DNA encoding a protein of interest.

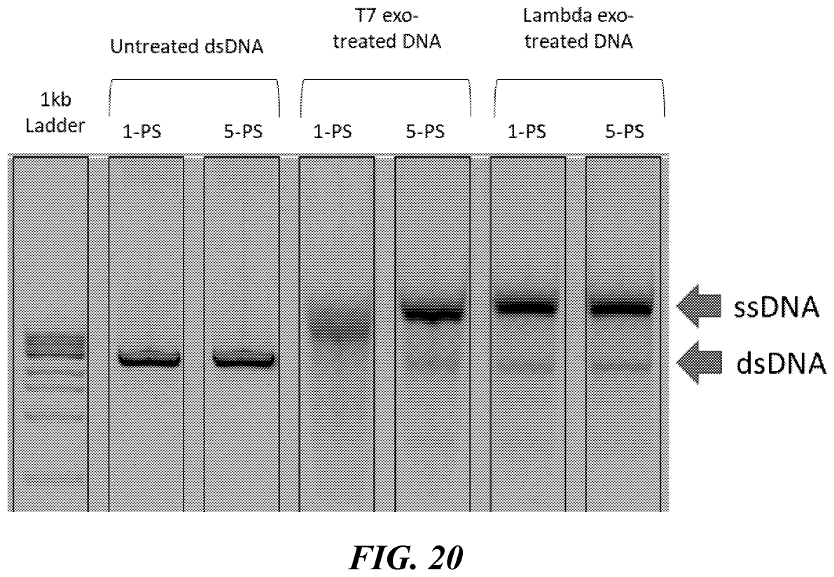

FIG. 20 shows digestion of double-stranded DNA (dsDNA) containing either one or five 5′ phosphorothioate (PS)-containing nucleotides with lambda exonuclease or T7 exonuclease. A 2.7 kb GFP-encoding dsDNA with either 1-PS (lane 2) or 5-PS (lane 3) were each treated with either T7 exonuclease (lanes 4 and 5) or lambda exonuclease (lanes 6 and 7).

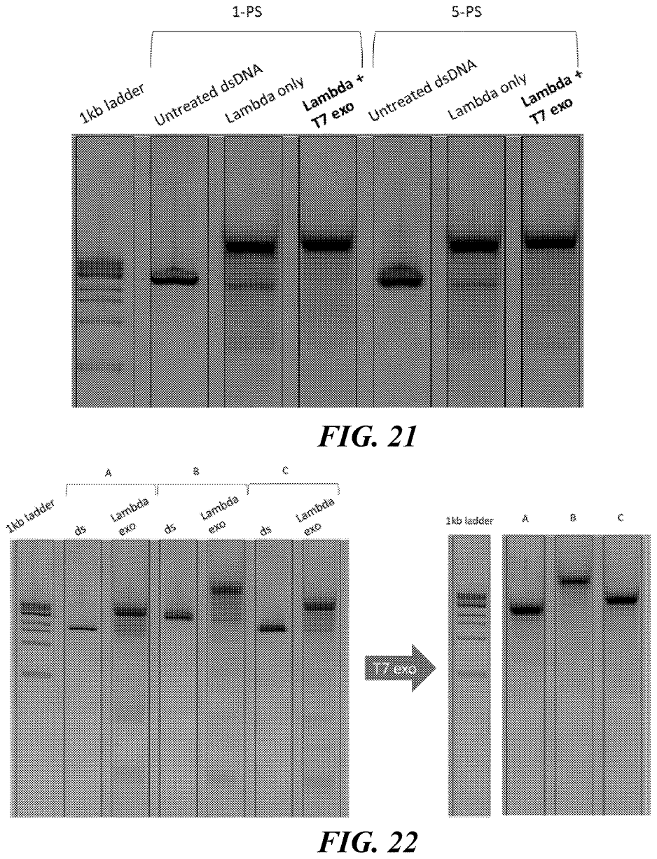

FIG. 21 shows that treatment with T7 exonuclease following lambda exonuclease digestion removed virtually all residual double-stranded product. 1-PS dsDNA (lane 2) was treated with lambda exonuclease (lane 3) and subsequently with T7 exonuclease (lane 4). 5-PS dsDNA was treated in parallel (lanes 5-7).

Table 5 below outlines the GC-content of single-stranded DNA donors A, B, and C.

TABLE 5
GC Contents

| | % GC content | Length of | Total |
|---|---|---|---|
| ssDNA | in GC-rich | GC-rich | ssDNA |
| donor | region | region | length |
| A | 75% | 456 bp | 1790 bp |
| B | 75% | 761 bp | 3420 bp |
| C | 75% | 761 bp | 2095 bp |

FIG. 22 shows on the left lambda exonuclease digestion of dsDNA with GC-rich regions. This gel shows dsDNA (“ds”) from samples A, B and C and the corresponding lambda exonuclease digests (“lambda exo”). FIG. 22 shows on the right that subsequent treatment with T7 exonuclease eliminated residual dsDNA. A, B, and C lambda exonuclease digests were quenched and then treated with T7 exonuclease. This gel displays ssDNA generated post-lambda exonuclease and post-T7 exonuclease digests.

Table 6 below describes the sequence elements, length, and yield of donor single-stranded DNA sequences. Sequences consist of the following: 90 bp of 5′ AAVS1 homology, promoter (SFC or EF1α), coding sequence, polyadenylation site, and 90 bp of 3′ AAVS1 homology. After purification, all samples were >1 μg/μL with no further concentration required.

TABLE 6
Donor DNA Sequences Characteristics

| | | Length | Yield |
|---|---|---|---|
| Sample | Sequence Elements | (kb) | (μg) |
| 1 | 5′AAVS1h90-SFC-GFP-wpre-SpA-AAVS1h90 3′ | 1.6 | 18.3 |
| 2 | 5′AAVS1h90-SFC-IDO1-SpA-AAVS1h90 3′ | 1.8 | 19.9 |
| 3 | 5′AAVS1h90-SFC-ROR1-CAR-SpA-AAVS1h90 3′ | 2.0 | 27.3 |
| 4 | 5′AAVS1h90-SFC-ROR1-CAR-GFP-SpA-AAVS1h90 3′ | 2.8 | 36.5 |
| 5 | 5′AAVS1h90-EF1α-IL7-IL15-hGHpA-AAVS1h90 3′ | 2.9 | 15.3 |

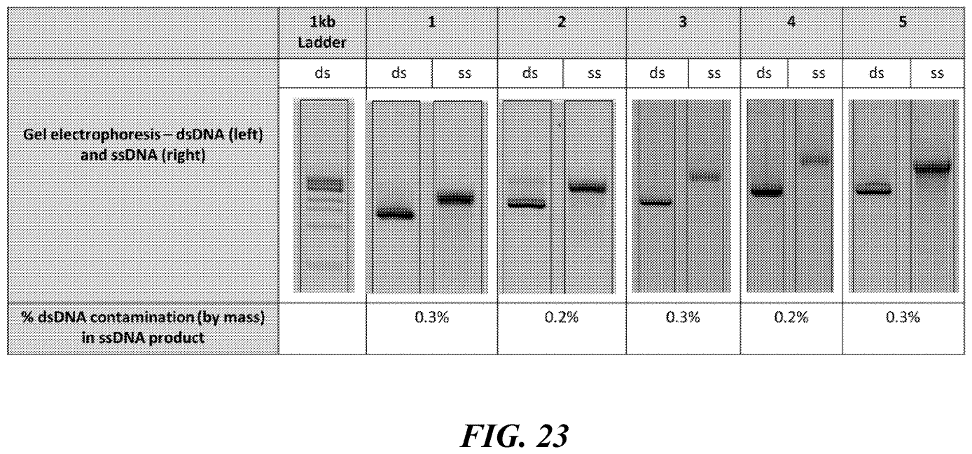

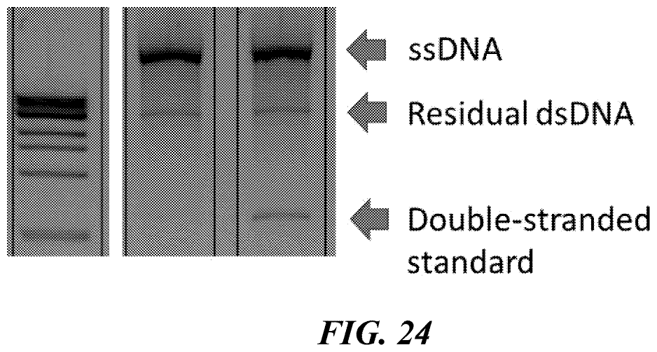

FIG. 23 shows a gel showing a side-by-side comparison of precursor dsDNA and purified ssDNA. Gels were loaded with double stranded (“ds”) DNA (left) and the corresponding enzymatically synthesized single-stranded (“ss”) DNA (right). Percent dsDNA contamination, by mass, was calculated using the method outlined in FIG. 24. For each sample 1-5, respectively, the percentage of dsDNA contamination was 0.3%, 0.2%, 0.3%, 0.2%, and 0.3%. In other words, double-stranded DNA comprising 5′ phosphorothioate (PS)-containing nucleotides is generally completely digested by sequential lambda exonuclease or T7 exonuclease whereas single-stranded DNA comprising 5′ phosphorothioate (PS)-containing nucleotides remained intact.

FIG. 24 shows a method of calculating percentage, by mass, of residual dsDNA in ssDNA samples. A 3.1 kb ssDNA sample (lane 2) was loaded at a high mass such that residual dsDNA (lower band) was detectable. The same ssDNA sample was co-loaded with a double-stranded DNA standard of known mass (lane 3). The intensity ratio of the residual dsDNA and the double-stranded standard enabled calculation of the former, which could then be expressed as a percentage of the total ssDNA sample mass. Again, double-stranded DNA comprising 5′ phosphorothioate (PS)-containing nucleotides is generally completely digested by sequential lambda exonuclease or T7 exonuclease whereas single-stranded DNA comprising 5′ phosphorothioate (PS)-containing nucleotides remained intact.

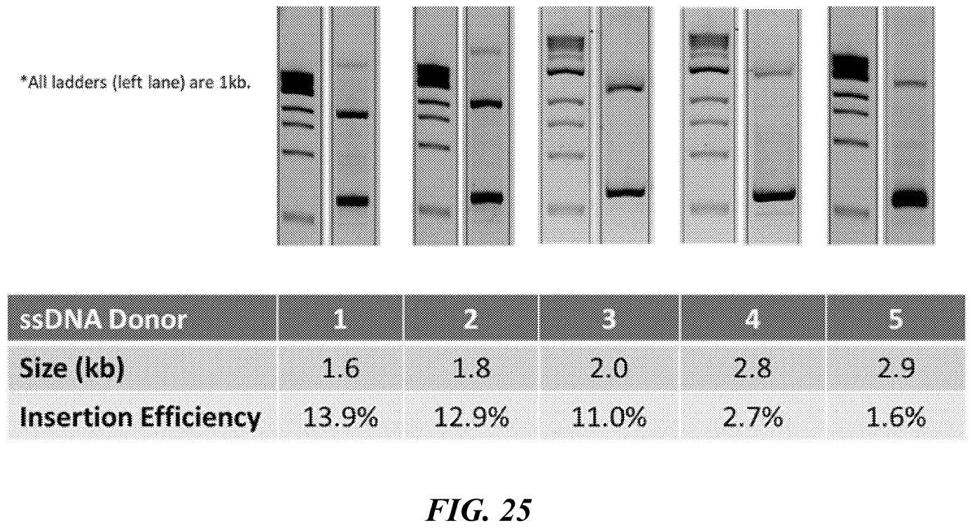

FIG. 25 shows the insertion efficiencies of ssDNA donors in iPS cells. iPS cells were co-electroporated with ssDNA donors (synthesized using the method described in this example) and mRNA encoding gene-editing proteins (disclosed elsewhere herein) and targeting the AAVS1 safe harbor locus. PCR was used to evaluate insertion efficiency, which was calculated using the intensity ratios of inserted (upper) to uninserted (lower) bands. The insertion efficiency of samples 1-5, respectively, was 13.9%, 12.9%, 11.0%, 2.7%, and 1.6%.

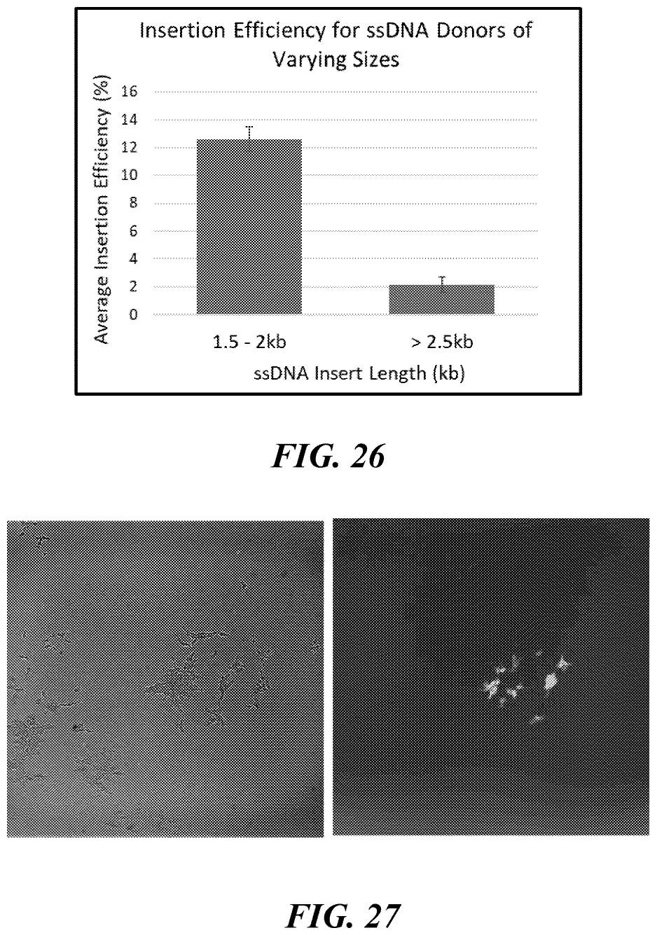

FIG. 26 shows that the insertion efficiency is higher for shorter (<2 kb) than for longer (>2.5 kb) donor ssDNA. The five ssDNA donors evaluated were grouped according to size (1.5-2 kb, left, and >2.5 kb, right). Average insertion efficiency (y-axis) is shown as a function of donor ssDNA length (x-axis). Bars represent standard error, and p=0.003.

FIG. 27 shows gene expression in iPS cells electroporated with a 2.5 kb ssDNA GFP-expressing template. iPS cells were imaged 6 days post electroporation with a GFP-encoding ssDNA template and AAVS1-targeting mRNA encoding gene editing protein of the present disclosure. Brightfield (left) and GFP (right) images are shown.

These data demonstrate that double-stranded DNA comprising 5′ phosphorothioate (PS)-containing nucleotides can be nearly completely digested by sequential lambda exonuclease or T7 exonuclease, whereas single-stranded DNA comprising 5′ phosphorothioate (PS)-containing nucleotides remained intact. By using this method, high purity single-stranded DNA can rapidly be made and then used, e.g., as a single stranded repair template for gene-editing.

In this example, dsDNA was PCR-amplified from plasmid templates using standard 5′ primers (which have the standard 5′ phosphate); this amplified strand was intended for digestion. dsDNA was also PCR-amplified using a modified primer (which had one to five 5′ phosphorothioate-containing nucleotides); this dsDNA was intended as the protein-encoding strand. The resulting PCR products were treated with lambda exonuclease, which preferentially digests strands with a 5′ phosphate group, to yield a single-stranded product. To minimize degradation of the ssDNA, a short, double-stranded oligonucleotide (dsDecoy) was included in the reaction, for which lambda exonuclease has higher affinity than ssDNA. Reactions were further treated with the less processive T7 exonuclease to eliminate residual dsDNA not digested by lambda exonuclease, yielding concentrated (>1 μg/μL) ssDNA. Gel electrophoresis analysis of five products ranging from 1.4 kb to 3.3 kb revealed a single, sharp band in the region of interest. Four of the ssDNA products contained less than 0.3% residual dsDNA by mass, as determined by gel electrophoresis using a double-stranded DNA standard of known concentration. The fifth, 3.3 kb ssDNA product contained 1.10% dsDNA by mass.

To test their utility as knock-in donors, ssDNA products were co-electroporated into hiPS cells with mRNA encoding gene-editing proteins and which targeted the AAVS1 safe harbor locus. Insertion efficiencies were 67.8% for a 1.2 kb donor, 8.6% for a 2 kb donor encoding a ROR1 CAR, and 2.7% for a 2.8 kb donor encoding green fluorescent protein. Notably, the presence of the 5′-phosphorothioate-containing nucleotides in the repair templates did not affect insertion frequency nor did they affect the gene's activity once integrated.

Although a single phosphorothioate-containing nucleotide in a DNA strand was sufficient to resist lambda exonuclease digestion and preserve single-stranded DNA for later use, a primer may include more phosphorothioate-containing nucleotides, e.g., 2, 3, 4, 5, 6, 7, 8, 9, 10 or more. Without wishing to bound by theory, more phosphorothioate-containing nucleotides would further protect the ssDNA from undesired digestion.

In summary, the data disclosed herein demonstrate a method for high-yield synthesis of ssDNA suitable for cellular applications, including mRNA gene editing-mediated knock-in iPS cells. Overall, this platform is useful in the development of a plurality of gene-editing therapeutics.

In summary, the data in this Example outlines a method for high-yield synthesis of single-stranded DNA (ssDNA) suitable for cellular applications, including gene editing, e.g., knock-ins in the genome of iPS cells. The present disclosure demonstrates that quality ssDNA can be synthesized from double-stranded DNA (dsDNA) via treatment with both lambda exonuclease and T7 exonuclease, and that a single phosphorothioate-containing nucleotide is sufficient to protect the coding strand from enzymatic activity. Additionally, this approach effectively synthesized ssDNA from dsDNA containing GC-rich regulatory elements, which could be relevant to cellular applications and experiments. The present disclosure also demonstrates that upon electroporation into iPS cells, ssDNA synthesized using this method can be inserted and expressed. Overall, the platform presented herein will prove useful in the development of gene-editing therapeutics.

### Example 4: Differentiation of Gene Edited iPSCs is Directed by Small-Molecule Inhibition of a Transgene-Encoded Protein

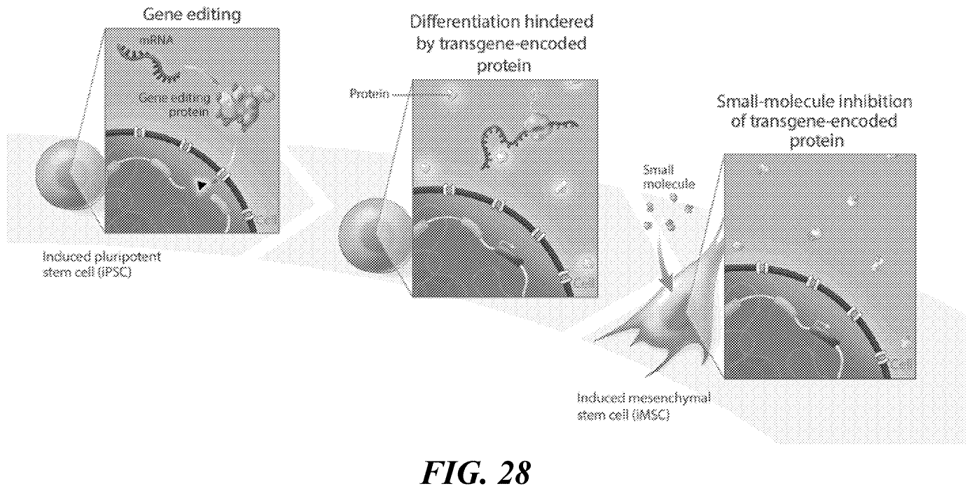

Indoleamine 2,3-dioxygenase 1 (IDO1) is an inducible, heme-containing enzyme that is critically involved in tryptophan catabolism and known to be a prominent immune regulator. Cell therapies with increased IDOI expression are of high interest for a variety of indications, including autoimmune disorders, inflammatory diseases, transplant recovery, and wound healing. In particular, iPSC-derived mesenchymal stem cells (iMSCs) engineered to overexpress IDO1 may be ideal for suppressing dysregulated immune cells simultaneously promoting expansion of regulatory T lymphocytes and the M1 (pro-inflammatory) to M2 (anti-inflammatory) polarization of macrophages. The data disclosed herein demonstrate the development of an iMSC cell line containing an IDO1 transgene under the control of a JeT promoter that was inserted into the AAVS1 safe-harbor locus in mRNA-reprogrammed iPSCs. A clonal population of edited cells (i.e., IDO1-iPSCs) was isolated using single-cell sorting. The IDO1-iPSCs were then differentiated to IDO1-iMSCs. During differentiation, the IDO1-Inserted cells showed an unexpected, cuboidal morphology and noticeable decreased proliferation rated relative to control iPSCs, possibly indicating that IDOI was interfering with the differentiation process. Accordingly, the use of small-molecule IDO1 inhibitors were investigated to determine whether such intervention could be a valid strategy for improving the differentiation. This process is illustrated in FIG. 28.

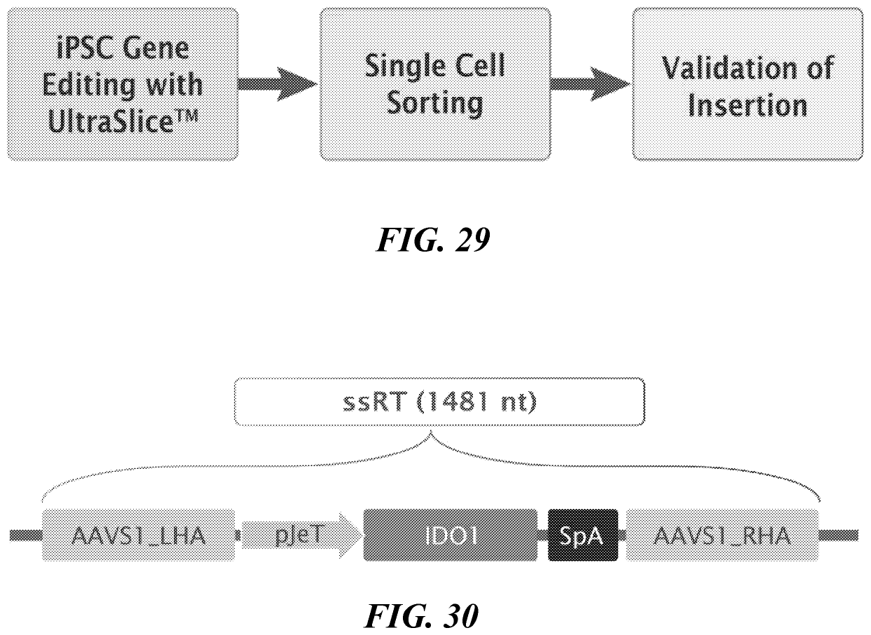

FIG. 29 is a flow chart illustrating the process of gene edited iPS cell line development. The iPSCs were first electroporated using mRNA encoding gene-editing proteins disclosed herein to insert a single stranded DNA (ssDNA) repair template for a gene of interest. Gene edited iPSCs were then single-cell sorted using a microfluidic cell sorter. When single-cell derived colonies recovered, biallelic insertion was confirmed by agarose gel electrophoresis and amplicon sequencing.

FIG. 30 shows a schematic of the IDO1-encoding single-stranded repair template (1481 nt), which was under control of the JeT promoter, terminated with a synthetic polyadenylation site, and bordered by both the left and right homology arms that are specific for the AAVS1 safe harbor locus.

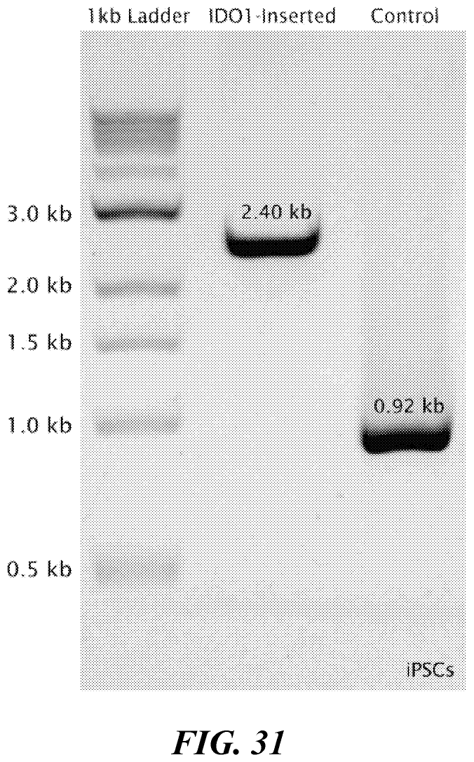

FIG. 31 is an agarose gel depicting bands of the AAVS1 amplicon for both Control iPSCs and IDO1-Inserted iPSCs, with a 1 kb ladder. The IDO1-inserted amplicon band was 2.40 kb in length, and the control amplicon band was 0.92 kb in length.

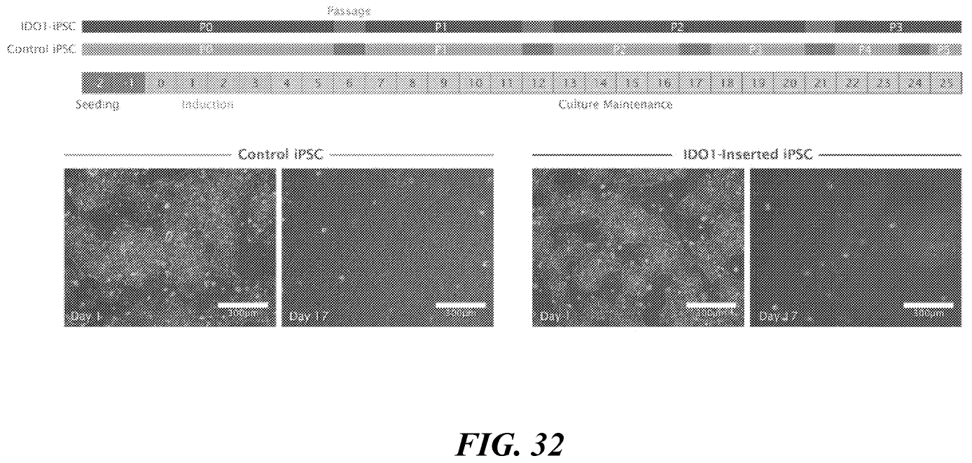

FIG. 32 shows that control and IDO1-Inserted iPSCs were differentiated into iMSCs using a commercially available kit. FIG. 32 (top) shows a schematic of the differentiation protocol. The cells with the IDO1 transgene proliferated at a slower relative to the Control iPSCs and therefore took more time to reach a necessary confluence level for passaging. Thus, the IDO1 transgene appeared to negatively affect the iPSC-to-iMSC differentiation. FIG. 32 (bottom) shows images of control and IDO1-inserted iPSCs at day 1 and at day 17 of the protocol.

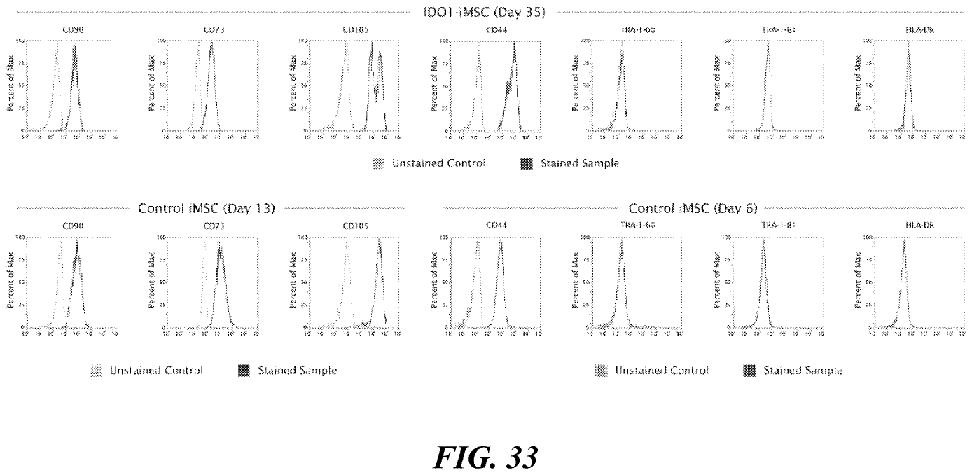

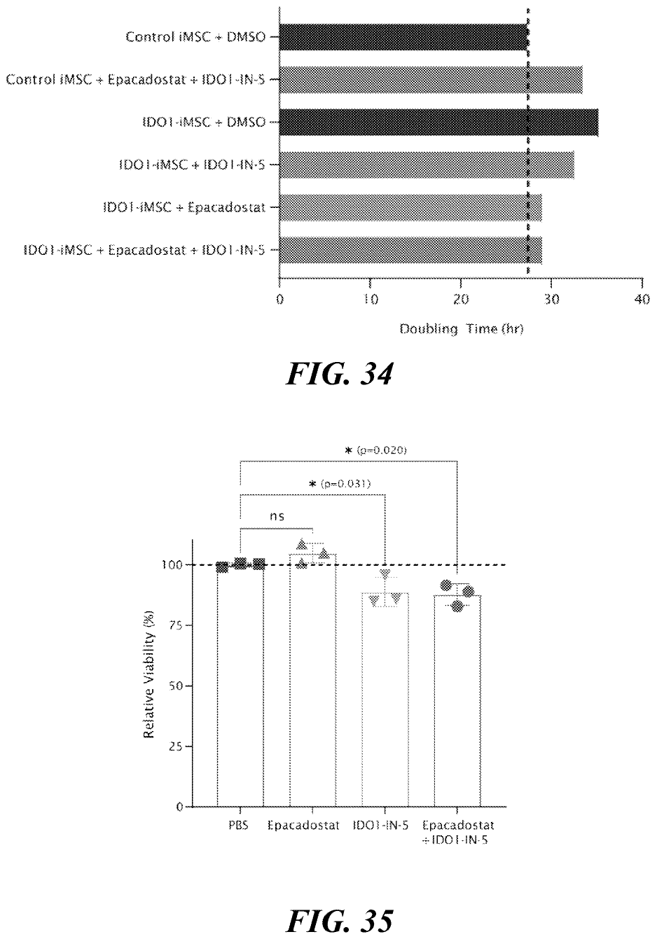

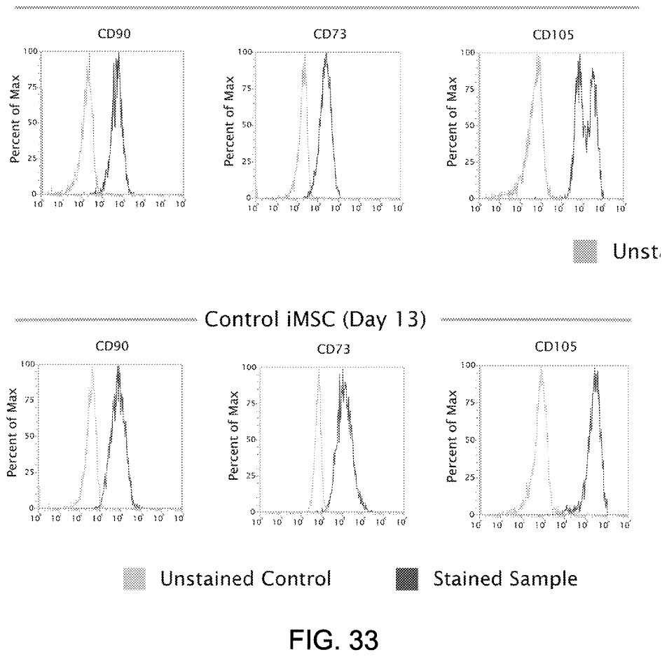

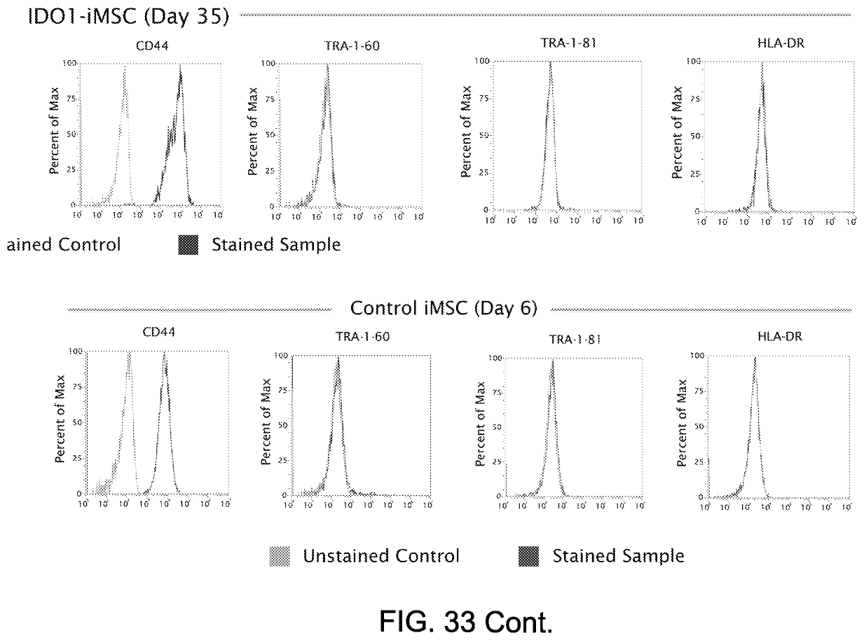

FIG. 33 shows flow cytometry histogram analysis of IDO1-iMSCs (top row) and Control iMSCs (bottom row). By day 35 of differentiation, IDO1-iMSCs showed phenotypic characteristics consistent with control iMSCs, albeit control iMSCs at day 17.

FIG. 34 is a graph showing doubling time of Control or IDO1-Inserted iMSCs after a five-day incubation with small molecule IDO1 inhibitors or controls. The dotted line indicates a doubling time of Control iMSCs with DMSO. Cells were seeded at day 35 post differentiation induction. All inhibitors were used at 10 μM concentrations in 0.5% DMSO. IDO1 Inserted iMSCs showed an increased proliferation rate with a six-hour reduction in doubling time when cultured in an IDO1 inhibitory environment. The population doubling time of IDO1 inhibited partly differentiated IDO1-iPSCs approached that of control iMSCs (29 h vs. 27 h). While both inhibitors could enhance proliferation independently, Epacadostat alone had a larger effect on population doubling time than the IDO1-IN-5 alone (6 h vs. 2 h reduction in doubling time), suggesting that the anti-inflammatory enzymatic function of IDO1 may be most responsible for decreased proliferation during differentiation of IDO1-iPSCs. Notably, the data presented in this Example also demonstrate that when administering both IDO1 inhibitors to non-engineered control iMSCs, the population doubling time increased relative to untreated test cultures (34 h versus 27 h), suggesting a possible minimum IDO1 protein level required for optimal iMSC culture.

FIG. 35 is a graph showing percent relative viability of iMSCs. The cytotoxicity of small-molecule IDO1 inhibitors was assessed by flow cytometry after a 48 hour incubation with Control iMSCs. Viability is shown relative to PBS controls. Epacadostat showed no significant difference relative to PBS, whereas IDO1-IN-5 and IDO1-IN-5+Epacadostat showed mild cytotoxicity (p=0.031 and p=0.020, respectively).

These data demonstrate the development of an iMSC cell line containing an IDO1 transgene under the control of a JeT promoter that was inserted into the AAVS1 safe-harbor locus in an mRNA-reprogrammed iPSCs. A clonal population of edited cells (i.e., IDO1-iPSCs) was isolated using single-cell sorting. Bi-allelic insertion of the IDO1 transgene was confirmed by amplicon sequencing. The IDO1-iPSCs were then differentiated into IDO1-iMSCs. During differentiation, the IDO1-iPSCs showed an unexpected, cuboidal morphology and with noticeably decreased proliferation rates relative to control iPSCs, possibly indicating that IDO1 was interfering with the differentiation process.

Two small-molecule IDO1 inhibitors, Epacadostat, which binds to holo (heme-bound) IDO1 and inhibits the enzymatic function of the protein and IDO1-IN-5, which binds to apo (heme-dissociated) IDO1 and inhibits IDO1-dependent cell signaling, were added to the culture for the final five days of differentiation. The addition of both IDO1 inhibitors (each at a 10 μM concentration) increased the proliferation rate of the partly-differentiated IDO1-iPSCs (six-hour reduction in doubling time) with a population doubling time approaching that of control iMSCs (29 h vs. 27 h). While both inhibitors enhanced proliferation independently, Epacadostat alone had a larger effect on population doubling time than the IDO1-IN-5 alone (six-hour vs. two-hour reduction in doubling time). Thus, the anti-inflammatory enzymatic function of IDO1 may be most responsible for decreased proliferation during differentiation of IDO1-iPSCs. Notably, when administering both IDO1 inhibitors to non-engineered control iMSCs, the population doubling time increased relative to untreated test cultures (34 hours versus 27 hours), indicating a possible minimum IDO1 protein level required for optimal iMSC culture. These results teach that small-molecule inhibition of transgene-encoded proteins may form a key element of directed differentiation process development for knock-in iPS cell lines, and are useful for the scale-up manufacturing of IDO1-iMSCs in particular.

In summary, the data presented in this Example demonstrate that engineered iPS cell lines that contain certain transgenes can exhibit altered growth characteristics and slower proliferation rates during differentiation, which may hinder scale-up and manufacturing process design. The data disclosed herein demonstrate that small-molecule inhibition of transgene-encoded proteins can enhance the growth rate of such gene-edited iPS cells during differentiation to iMSCs. Thus, the use of small-molecule inhibitors represents a promising new strategy to alleviate aberrant differentiation characteristics caused by the presence of a transgene in gene-edited iPSCs, which may prove useful for the generation of engineered iPSC-derived cell.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

### Numbered Embodiments

Embodiment 1. A method for creating a single-stranded break in a DNA site, the method comprising: (a) contacting the DNA with a first gene-editing protein comprising a nuclease domain (1) capable of binding to the DNA and forming a dimer with another nuclease domain, and (2) capable of creating a single-stranded break in a DNA site; and (b) contacting the DNA with a second gene-editing protein comprising a nuclease domain (1) capable of binding to the DNA and forming a dimer with another nuclease domain, and (2) incapable of creating a single-stranded break in a DNA site.
Embodiment 2. The method of embodiment 1, wherein the nuclease domain incapable of creating a single-stranded break in a DNA site comprises a mutation in its catalytic domain.
Embodiment 3. The method of embodiment 1 or 2, further comprising contacting the DNA with a single-stranded repair template.
Embodiment 4. The method of embodiment 3, wherein the single-stranded repair template integrates into the DNA at the single-stranded break.
Embodiment 5. A method for creating a single-stranded break in a DNA site of a cell, the method comprising contacting the cell with a pair of synthetic RNAs comprising a first synthetic RNA and a second synthetic RNA, wherein the first synthetic RNA encodes a gene-editing protein comprising a nuclease domain (1) capable of binding to the DNA and forming a dimer with another nuclease domain, and (2) capable of creating a single-stranded break in a DNA site of the cell, and wherein a second synthetic RNA encoding a gene-editing protein comprising a nuclease domain (1) capable of binding to the DNA and forming a dimer with another nuclease domain, and (2) incapable of creating a single-stranded break in a DNA site of the cell.
Embodiment 6. The method of embodiment 5, wherein the nuclease domain incapable of creating a single-stranded break in a DNA site comprises a mutation in its catalytic domain.
Embodiment 7. The method of embodiment 5 or 6, further comprising contacting the cell with a single-stranded repair template.
Embodiment 8. The method of embodiment 7, wherein the single-stranded repair template is DNA.
Embodiment 9. The method of embodiment 7 or 8, wherein the single-stranded repair template integrates into the DNA of the cell at the single-stranded break.
Embodiment 10. The method of any one of embodiments 1-9, wherein the nuclease domain incapable of creating a single-stranded break in a DNA site comprises a FokI domain.
Embodiment 11. The method of embodiment 10, wherein the FokI domain bears a mutation.
Embodiment 12. The method of embodiment 11, wherein the mutation is selected from the group consisting of D67A, D67N, D84A, and any combinations thereof, each of which is numbered in reference to the sequence of SEQ ID NO: 80.
Embodiment 13. The method of embodiment 12, wherein the mutation is a D67A mutation and the FokI domain comprises a sequence with at least 80%, 85%, 90%, 95%, 96%, 98%, or 99% sequence identity to SEQ ID NO: 81.
Embodiment 14. The method of embodiment 13, wherein the FokI domain is encoded by a nucleic acid comprising a sequence with at least 80%, 85%, 90%, 95%, 96%, 98%, or 99% sequence identity to SEQ ID NO: 85.
Embodiment 15. The method of embodiment 12, wherein the mutation is a D67N mutation and the FokI domain comprises a sequence with at least 80%, 85%, 90%, 95%, 96%, 98%, or 99% sequence identity to SEQ ID NO: 82.
Embodiment 16. The method of embodiment 15, wherein the FokI domain is encoded by a nucleic acid comprising a sequence with at least 80%, 85%, 90%, 95%, 96%, 98%, or 99%, or 100% sequence identity to SEQ ID NO: 86.
Embodiment 17. The method of embodiment 12, wherein the mutation is a D67A mutation and the FokI domain comprises a sequence with at least 80%, 85%, 90%, 95%, 96%, 98%, or 99%, or 100% sequence identity to SEQ ID NO: 83.
Embodiment 18. The method of embodiment 17, wherein the FokI domain is encoded by a nucleic acid comprising a sequence with at least 80%, 85%, 90%, 95%, 96%, 98%, or 99%, or 100% sequence identity to SEQ ID NO: 87.
Embodiment 19. The method of any one of embodiments 1-18, wherein the first gene editing protein and/or the second gene editing protein comprises a plurality of repeat sequences and at least one of the repeat sequences comprises the amino acid sequence: LTPvQVVAIAwxyz (SEQ ID NO: 16), wherein:
(a) v is Q, D or E, (b) w is S or N, (c) x is I, H, or N, (d) y is D, A, I, N, H, K, S, G or null, and (e) z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20).
Embodiment 20. The method of any one of embodiments 1-18, wherein the first gene editing protein and/or the second gene editing protein comprises a plurality of repeat sequences and at least one of the repeat sequences comprises the amino acid sequence: LTPvQVVAIAwxyzα (SEQ ID NO: 22), wherein: (a) v is Q, D or E, (b) w is S or N, (c) x is I, H, or N, (d) y is D, A, I, N, H, K, S, G or null, (e) z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), and (f) α is any four consecutive amino acids.
Embodiment 21. The method of embodiment 20, wherein a comprises at least one glycine (G) residue.
Embodiment 22. The method of embodiment 20 or 21, wherein a comprises at least one histidine (H) residue.
Embodiment 23. The method of any one of embodiments 20-22, wherein a comprises at least one histidine (H) residue at any one of positions 33, 34, or 35.
Embodiment 24. The method of any one of embodiments 20-23, wherein a comprises at least one aspartic acid (D) residue.
Embodiment 25. The method of any one of embodiments 20-24, wherein a comprises at least one, or two, or three of a glycine (G) residue, a histidine (H) residue, and an aspartic acid (D) residue.
Embodiment 26. The method of any one of embodiments 20-25, wherein a comprises one or more hydrophilic residues, optionally selected from: (a) a polar and positively charged hydrophilic amino acid, optionally selected from arginine (R) and lysine (K); (b) a polar and neutral of charge hydrophilic amino acid, optionally selected from asparagine (N), glutamine (Q), seine (S), threonine (T), proline (P), and cysteine (C); (c) a polar and negatively charged hydrophilic amino acid, optionally selected from aspartate (D) and glutamate (E), and (d) an aromatic, polar and positively charged hydrophilic amino acid, optionally selected from histidine (H).
Embodiment 27. The method of any one of embodiments 20-26, wherein a comprises one or more hydrophobic residues, optionally selected from: (a) a hydrophobic, aliphatic amino acid, optionally selected from glycine (G), alanine (A), leucine (L), isoleucine (I), methionine (M), and valine (V), and (b) a hydrophobic, aromatic amino acid, optionally selected from phenylalanine (F), tryptophan (W), and tyrosine (Y).
Embodiment 28. The method of any one of embodiments 20-27, wherein a is selected from GHGG (SEQ ID NO: 31), HGSG (SEQ ID NO: 32), HGGG (SEQ ID NO: 33), GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60).
Embodiment 29. The method of any one of embodiments 1-18, wherein the first gene editing protein and/or the second gene editing protein comprises: (a) a plurality of repeat sequences and at least one of the repeat sequences comprises the amino acid sequence: LTPvQVVAIAwxyzGHGG (SEQ ID NO: 17), wherein: “v” is Q, D or E, “w” is S or N, “x” is H, N, or I, “y” is D, A, I, N, G, H, K, S, G or null, and “z” is GGKQALETVQRLLPVLCQD (SEQ ID NO: 19) or GGKQALETVQRLLPVLCQA (SEQ ID NO: 20); and (b) a nuclease domain comprising a catalytic domain of a nuclease.
Embodiment 30. The method of any one of embodiments 1-18, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 31. The method of any one of embodiments 1-18, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 32. The method of any one of embodiments 1-18, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is any amino acid other than N, H and I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 33. The method of any one of embodiments 1-18, wherein the first gene editing protein and/or the second gene editing protein comprises a plurality of repeat sequences and at least one of the repeat sequences comprises the amino acid sequence: LTPvQVVAIAwIyzGHGG (SEQ ID NO: 88), wherein “v” is Q, D or E, “w” is S or N, “y” is any amino acid other than G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 34. The method of any one of embodiments 1-18, wherein the first gene editing protein and/or the second gene editing protein comprises a plurality of repeat sequences and at least one of the repeat sequences comprises the amino acid sequence: LTPvQVVAIAwIAzGHGG (SEQ ID NO: 71), wherein “v” is Q, D or E, “w” is S or N, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 35. The method of any one of embodiments 1-18, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 36. The method of any one of embodiments 1-18, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 37. The method of any one of embodiments 1-18, wherein the first gene editing protein and/or the second gene editing protein comprises a plurality of repeat sequences and at least one of the repeat sequences comprises the amino acid sequence: LTPvQVVAIAwx (SEQ ID NO: 67), wherein “v” is Q, D or E, “w” is S or N, and “x” is S, T or Q.
Embodiment 38. The method of any one of embodiments 1-18, wherein the first gene editing protein and/or the second gene editing protein comprises a plurality of repeat sequences and at least one of the repeat sequences comprises the amino acid sequence: LTPvQVVAIAwxy (SEQ ID NO: 72), wherein “v” is Q, D or E, “w” is S or N, “x” is S, T or Q, and “y” is selected from: D, A, I, N, H, K, S, and G.
Embodiment 39. A pair of synthetic RNAs comprising a first synthetic RNA and a second synthetic RNA, wherein the first synthetic RNA encodes a gene-editing protein comprising a nuclease domain (1) capable of binding to the DNA and forming a dimer with another nuclease domain, and (2) capable of creating a single-stranded break in a DNA site, and wherein the second synthetic RNA encodes a gene-editing protein comprising a nuclease domain (1) capable of binding to the DNA and forming a dimer with another nuclease domain, and (2) incapable of creating a single-stranded break in a DNA site.
Embodiment 40. The pair of synthetic RNAs of embodiment 39, wherein the nuclease domain incapable of creating a single-stranded break in a DNA site comprises a FokI domain.
Embodiment 41. The pair of synthetic RNAs of embodiment 39 or 40, wherein the FokI domain bears a mutation.
Embodiment 42. The pair of synthetic RNAs of embodiment 41, wherein the mutation is selected from the group consisting of D67A, D67N, D84A, and any combinations thereof, each of which is numbered in reference to the sequence of SEQ ID NO: 80.
Embodiment 43. The pair of synthetic RNAs of embodiment 41, wherein the mutation is a D67A mutation and the FokI domain comprises a sequence with at least 80%, 85%, 90%, 95%, 96%, 98%, or 99% sequence identity to SEQ ID NO: 81.
Embodiment 44. The pair of synthetic RNAs of embodiment 43, wherein the FokI domain is encoded by a nucleic acid comprising a sequence with at least 80%, 85%, 90%, 95%, 96%, 98%, or 99% sequence identity to SEQ ID NO: 85.
Embodiment 45. The pair of synthetic RNAs of embodiment 41, wherein the mutation is a D67N mutation and the FokI domain comprises a sequence with at least 80%, 85%, 90%, 95%, 96%, 98%, or 99% sequence identity to SEQ ID NO: 82.
Embodiment 46. The pair of synthetic RNAs of embodiment 45, wherein the FokI domain is encoded by a nucleic acid comprising a sequence with at least 80%, 85%, 90%, 95%, 96%, 98%, or 99%, or 100% sequence identity to SEQ ID NO: 86.
Embodiment 47. The pair of synthetic RNAs of embodiment 41, wherein the mutation is a D67A mutation and the FokI domain comprises a sequence with at least 80%, 85%, 90%, 95%, 96%, 98%, or 99%, or 100% sequence identity to SEQ ID NO: 83.
Embodiment 48. The pair of synthetic RNAs of embodiment 47, wherein the FokI domain is encoded by a nucleic acid comprising a sequence with at least 80%, 85%, 90%, 95%, 96%, 98%, or 99%, or 100% sequence identity to SEQ ID NO: 87.
Embodiment 49. The pair of synthetic RNAs of any one of embodiments 39 to 48, wherein the first gene editing protein and/or the second gene editing protein comprises a plurality of repeat sequences and at least one of the repeat sequences comprises the amino acid sequence: LTPvQVVAIAwxyz (SEQ ID NO: 16), wherein: (a) v is Q, D or E, (b) w is S or N, (c)
x is I, H, or N, (d) y is D, A, I, N, H, K, S, G or null, and (e) z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 29) or GGKQALETVQRLLPVLCQA (SEQ ID NO: 20).
Embodiment 50. The pair of synthetic RNAs of any one of embodiments 39 to 48, wherein the first gene editing protein and/or the second gene editing protein comprises a plurality of repeat sequences and at least one of the repeat sequences comprises the amino acid sequence: LTPvQVVAIAwxyzα (SEQ ID NO: 22), wherein: (a) v is Q, D or E, (b) w is S or N, (c)
x is I, H, or N, (d) y is D, A, I, N, H, K, S, G or null, (e) z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), and (f) α is any four consecutive amino acids.
Embodiment 51. The pair of synthetic RNAs of embodiment 50, wherein a comprises at least one glycine (G) residue.
Embodiment 52. The pair of synthetic RNAs of embodiment 50 or 51, wherein a comprises at least one histidine (H) residue.
Embodiment 53. The pair of synthetic RNAs of any one of embodiments 50-52, wherein a comprises at least one histidine (H) residue at any one of positions 33, 34, or 35.
Embodiment 54. The pair of synthetic RNAs of any one of embodiments 50-53, wherein a comprises at least one aspartic acid (D) residue.
Embodiment 55. The pair of synthetic RNAs of any one of embodiments 50-54, wherein a comprises at least one, or two, or three of a glycine (G) residue, a histidine (H) residue, and an aspartic acid (D) residue.
Embodiment 56. The pair of synthetic RNAs of any one of embodiments 50-55, wherein a comprises one or more hydrophilic residues, optionally selected from: (a) a polar and positively charged hydrophilic amino acid, optionally selected from arginine (R) and lysine (K); (b) a polar and neutral of charge hydrophilic amino acid, optionally selected from asparagine (N), glutamine (Q), seine (S), threonine (T), proline (P), and cysteine (C); (c) a polar and negatively charged hydrophilic amino acid, optionally selected from aspartate (D) and glutamate (E), and (d) an aromatic, polar and positively charged hydrophilic amino acid, optionally selected from histidine (H).
Embodiment 57. The pair of synthetic RNAs of any one of embodiments 50-56, wherein a comprises one or more hydrophobic residues, optionally selected from: (a) a hydrophobic, aliphatic amino acid, optionally selected from glycine (G), alanine (A), leucine (L), isoleucine (I), methionine (M), and valine (V), and (b) a hydrophobic, aromatic amino acid, optionally selected from phenylalanine (F), tryptophan (W), and tyrosine (Y).
Embodiment 58. The pair of synthetic RNAs of any one of embodiments 50-57, wherein a is selected from GHGG (SEQ ID NO: 31), HGSG (SEQ ID NO: 32), HGGG (SEQ ID NO: 33), GGHD (SEQ ID NO: 34), GAHD (SEQ ID NO: 35), AHDG (SEQ ID NO: 36), PHDG (SEQ ID NO: 37), GPHD (SEQ ID NO: 38), GHGP (SEQ ID NO: 39), PHGG (SEQ ID NO: 40), PHGP (SEQ ID NO: 41), AHGA (SEQ ID NO: 42), LHGA (SEQ ID NO: 43), VHGA (SEQ ID NO: 44), IVHG (SEQ ID NO: 45), IHGM (SEQ ID NO: 46), RHGD (SEQ ID NO: 47), RDHG (SEQ ID NO: 48), RHGE (SEQ ID NO: 49), HRGE (SEQ ID NO: 50), RHGD (SEQ ID NO: 47), HRGD (SEQ ID NO: 51), GPYE (SEQ ID NO: 52), NHGG (SEQ ID NO: 53), THGG (SEQ ID NO: 54), GTHG (SEQ ID NO: 21), GSGS (SEQ ID NO: 56), GSGG (SEQ ID NO: 57), GGGG (SEQ ID NO: 58), GRGG (SEQ ID NO: 59), and GKGG (SEQ ID NO: 60).
Embodiment 59. The pair of synthetic RNAs of any one of embodiments 39-48, wherein the first gene editing protein and/or the second gene editing protein comprises: (a) a plurality of repeat sequences and at least one of the repeat sequences comprises the amino acid sequence: LTPvQVVAIAwxyzGHGG (SEQ ID NO: 17), wherein: “v” is Q, D or E, “w” is S or N, “x” is H, N, or I, “y” is D, A, I, N, G, H, K, S, G or null, and “z” is GGKQALETVQRLLPVLCQD (SEQ ID NO: 19) or GGKQALETVQRLLPVLCQA (SEQ ID NO: 20); and (b) a nuclease domain comprising a catalytic domain of a nuclease.
Embodiment 60. The pair of synthetic RNAs of any one of embodiments 39-48, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 61. The pair of synthetic RNAs of any one of embodiments 39-48, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is N, H or I, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 62. The pair of synthetic RNAs of any one of embodiments 39-48, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is any amino acid other than N, H and I, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 63. The pair of synthetic RNAs of any one of embodiments 39-48, wherein the first gene editing protein and/or the second gene editing protein comprises a plurality of repeat sequences and at least one of the repeat sequences comprises the amino acid sequence: LTPvQVVAIAwIyzGHGG (SEQ ID NO: 88), wherein “v” is Q, D or E, “w” is S or N, “y” is any amino acid other than G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 64. The pair of synthetic RNAs of any one of embodiments 39-48, wherein the first gene editing protein and/or the second gene editing protein comprises a plurality of repeat sequences and at least one of the repeat sequences comprises the amino acid sequence: LTPvQVVAIAwIAzGHGG (SEQ ID NO: 71), wherein “v” is Q, D or E, “w” is S or N, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 65. The pair of synthetic RNAs of any one of embodiments 39-48, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is any amino acid or no amino acid, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 66. The pair of synthetic RNAs of any one of embodiments 39-48, wherein the first gene editing protein and/or the second gene editing protein comprises a plurality of repeat sequences and at least one of the repeat sequences comprises the amino acid sequence: LTPvQVVAIAwxyzGHGG (SEQ ID NO: 17), wherein “v” is Q, D or E, “w” is S or N, “x” is S, T or Q, “y” is selected from: D, A, I, N, H, K, S, and G, and “z” is GGRPALE (SEQ ID NO: 23), GGKQALE (SEQ ID NO: 24), GGKQALETVQRLLPVLCQD (SEQ ID NO: 19), GGKQALETVQRLLPVLCQA (SEQ ID NO: 20), GKQALETVQRLLPVLCQD (SEQ ID NO: 69) or GKQALETVQRLLPVLCQA (SEQ ID NO: 68).
Embodiment 67. The pair of synthetic RNAs of any one of embodiments 39-48, wherein the first gene editing protein and/or the second gene editing protein comprises a plurality of repeat sequences and at least one of the repeat sequences comprises the amino acid sequence: LTPvQVVAIAwx (SEQ ID NO: 67), wherein “v” is Q, D or E, “w” is S or N, and “x” is S, T or Q.
Embodiment 68. The pair of synthetic RNAs of any one of embodiments 39-48, wherein the first gene editing protein and/or the second gene editing protein comprises a plurality of repeat sequences and at least one of the repeat sequences comprises the amino acid sequence: LTPvQVVAIAwxy (SEQ ID NO: 72), wherein “v” is Q, D or E, “w” is S or N, “x” is S, T or Q, and “y” is selected from: D, A, I, N, H, K, S, and G.
Embodiment 69. A composition comprising the pair of synthetic RNAs of any one of embodiments 39-68.
Embodiment 70. The composition of embodiment 69, wherein the composition further comprises a single-stranded DNA template.
Embodiment 71. A kit comprising the pair of synthetic RNAs of any one of embodiments 39-68 and a single-stranded DNA template.
Embodiment 72. A method for activating gene expression, the method comprising: obtaining a cell comprising a transgene which expresses a protein of interest under the control of a promoter which is active during a defined period of a cell's differentiation and, when the cell's differentiation state is before or after the defined period during which the promoter is active, contacting the cell with a compound that demethylates DNA.
Embodiment 73. The method of embodiment 72, wherein the promoter has reduced activity as the cell differentiates, and wherein the method comprises contacting the cell with the compound that demethylates DNA during differentiation to reactivate the promoter.
Embodiment 74. The method of embodiment 72, wherein the promoter is inactive once the cell has differentiated, and wherein the method comprises contacting the cell with the compound that demethylates DNA after the cell has differentiated to reactivate the promoter.
Embodiment 75. The method of embodiment 72, wherein the promoter is active as the cell differentiates, and wherein the method comprises contacting the cell with the compound that demethylates DNA before the cell begins to differentiate to activate the promoter.
Embodiment 76. The method of any one of embodiments 72-75, wherein the compound that demethylates DNA is a selective histone deacetylase (HDAC) inhibitor.
Embodiment 77. The method of embodiment 76, wherein the selective HDAC inhibitor is selected from the group consisting of: trapoxin, suberanilohydroxamic acid, valproate, and romidepsin, and combinations thereof, optionally the selective HDAC inhibitor is Trichostatin A or a functional derivative thereof.
Embodiment 78. The method of any one of embodiments 72-77, wherein activating or reactivating the promoter provides expression of protein of interest at an atypical period in the cell's differentiation.
Embodiment 79. The method of embodiment 78, wherein expression of the protein of interest at the atypical period in the cell's differentiation changes fate of the cell and/or changes activity of the cell.
Embodiment 80. The method of embodiment 79, wherein changing the fate of the cell comprising differentiating the cell into a cell type or cell subtype for which it was not previously destined.
Embodiment 81. The method of any one of embodiments 72-80, wherein the cell is an induced pluripotent stem cell (iPSC) which has not begun differentiating into an induced mesenchymal stem cell (iMSC).
Embodiment 82. The method of any one of embodiments 72-80, wherein the cell is an induced pluripotent stem cell (iPSC) which is differentiating into an induced mesenchymal stem cell (iMSC).
Embodiment 83. The method of any one of embodiments 72-80, wherein the promoter is a JeT promoter.
Embodiment 84. A method for producing single-stranded DNA, the method comprising:
(a) obtaining a double-stranded DNA, which comprises a strand comprising at least one phosphorothioate-containing nucleotide and a strand lacking a phosphorothioate-containing nucleotide, and (b) contacting the double-stranded DNA with one or more nucleases which digest the strand lacking the phosphorothioate-containing nucleotide, thereby obtaining a single stranded DNA.
Embodiment 85. The method of embodiment 82, wherein the double-stranded DNA is synthesized by PCR using a standard 5′ primer lacking a phosphorothioate-containing nucleotide, and a 5′ primer comprising one or more phosphorothioate-containing nucleotides, or wherein the method further comprises synthesizing the double-stranded DNA by PCR using a standard 5′ primer lacking the phosphorothioate-containing nucleotide and a 5′ primer comprising one or more phosphorothioate-containing nucleotides.
Embodiment 86. The method of embodiment 84 or 85, wherein the one or more nucleases comprises lambda exonuclease or T7 exonuclease.
Embodiment 87. The method of embodiment 84 or 85, wherein the one or more nucleases comprises lambda exonuclease and T7 exonuclease.
Embodiment 88. The method of embodiment 87, wherein the DNA is contacted with lambda exonuclease before contacting with T7 exonuclease.
Embodiment 89. The method of any one of embodiments 84-88, wherein the strand comprising at least one phosphorothioate-containing nucleotide resists digestion by the one or more nucleases.
Embodiment 90. The method of any one of embodiments 84-88, wherein the method reduces the amount of double-stranded contamination in a product comprising the single-stranded DNA by at least 95%, e.g., at least 99%.
Embodiment 91. The method of any one of embodiments 84-88, wherein the double stranded DNA comprises at least 50% GC base pairs.
Embodiment 92. The method of any one of embodiments 84-91, wherein the single stranded DNA is used as a repair template in the method of any one of embodiments 8-38.
Embodiment 93. A single stranded DNA comprising at least one phosphorothioate-containing nucleotide and obtained by the method of any one of embodiments 84-92.
Embodiment 94. A method for manufacturing iPSC-derived differentiated cells that are engineered to overexpress IDO1, the method comprising: obtaining an iPSC comprising a genetic modification which results in overexpression of IDO1 in the iPSC, culturing the iPSC under conditions that promote differentiation of the iPSC, and contacting the iPSC with one or more IDO1 inhibitors.
Embodiment 95. The method of embodiment 94, wherein the iPSC comprising the genetic modification which results in overexpression of IDO1 in the iPSC has delayed or non-existent differentiation absent contact with the one or more IDO1 inhibitors.
Embodiment 96. The method of embodiment 94 or 95, wherein the iPSC comprising the genetic modification which results in overexpression of IDO1 in the iPSC has increased population doubling time absent contact with the one or more IDO1 inhibitors.
Embodiment 97. The method of any one of embodiments 94-96, wherein the contacting the iPSC with the one or more IDO1 inhibitors permits differentiation of the iPSC and/or reduces population doubling time of the iPSC.
Embodiment 98. The method of embodiment 97, wherein the method comprises differentiating the iPSC into a cell of the lymphoid lineage, a cell of the myeloid lineage, a hematopoietic stem cell, or a mesenchymal stromal/stem cell (MSC).
Embodiment 99. The method of any one of embodiments 94-98, wherein the one or more IDO1 inhibitors comprise Epacadostat and/or IDO1-IN-5.
Embodiment 100. The method of any one of embodiments 94-98, wherein the one or more IDO1 inhibitors comprise both Epacadostat and IDO1-IN-5.
Embodiment 101. A method for suppressing dysregulated immune cells while simultaneously promoting expansion of regulatory T lymphocytes and the M1 (pro-inflammatory) to M2 (anti-inflammatory) polarization of macrophages in a subject in need thereof, the method comprising administering to the subject a therapeutically effective amount of an engineered cell, wherein the engineered cell is a cell of the lymphoid lineage, a cell of the myeloid lineage, a hematopoietic stem cell, or a mesenchymal stromal/stem cell (MSC), and the engineered cell comprises a genetic modification that results in overexpression of IDO1 in the engineered cell.
Embodiment 102. The method of embodiment 101, wherein the engineered cell is derived from an iPSC.
Embodiment 103. A pharmaceutical composition comprising a therapeutically effective amount of an engineered cell, wherein the engineered cell is a cell of the lymphoid lineage, a cell of the myeloid lineage, a hematopoietic stem cell, or a mesenchymal stromal/stem cell (MSC), and the engineered cell comprises a genetic modification that results in overexpression of IDO1 in the engineered cell.
Embodiment 104. A solid support comprising a therapeutically effective amount of mesenchymal stromal/stem cells (MSCs), wherein the MSCs comprise a genetic modification resulting in overexpression of IDO1 in the MSCs, and are derived from an iPSC.
Embodiment 105. A solid support comprising a therapeutically effective amount of the mesenchymal stromal/stem cells (MSCs) manufactured by the method of any one of embodiments 94-100.
Embodiment 106. The solid support of embodiment 104 or 105, wherein the solid support comprises a patch.
Embodiment 107. The solid support of embodiment 104 or 105, wherein the solid support comprises a sheet comprising collagen.
Embodiment 108. The solid support of any one of embodiments 104-107, wherein the solid support further comprises a polymer.
Embodiment 109. The solid support of any one of embodiments 104-108, wherein the solid support further comprises a hydrogel.
Embodiment 110. A method for treating a damaged organ, the method comprising contacting a damaged portion of the damaged organ with the solid support of any one of embodiments 104-109.
Embodiment 111. A method for treating a damaged organ, the method comprising contacting a damaged portion of the damaged organ with a solid support comprising a therapeutically effective amount of mesenchymal stromal/stem cells (MSCs), wherein the MSCs comprise a genetic modification resulting in overexpression of IDO1 in the MSCs, and are derived from an iPSC.

1-111. (canceled)

112. A method for generating a single-strand break in a target deoxyribonucleic acid (DNA), the method comprising contacting a cell comprising the target DNA with a first synthetic ribonucleic acid (RNA) and a second synthetic RNA,
wherein the first synthetic RNA encodes a first gene-editing protein and the second synthetic RNA encodes a second gene-editing protein,
wherein the first gene-editing protein and the second gene-editing protein are capable of forming a dimer, and
wherein the dimer is capable of creating a single-strand break, thereby generating the single-strand break.

113. The method of claim 112, wherein the first-gene editing protein or the second gene-editing protein comprises a mutation.

114. The method of claim 112, further comprising contacting the cell with a single-stranded repair template.

115. The method of claim 114, wherein the single-stranded repair template comprises DNA.

116. The method of claim 115, wherein the single-stranded repair template integrates into DNA of the cell at the single-strand break.

117. The method of claim 113, wherein the first gene-editing protein or the second gene-editing protein comprises a FokI domain.

118. The method of claim 117, wherein the FokI domain comprises the mutation.

119. The method of claim 118, wherein the mutation is selected from the group consisting of: D67A, D67N, D84A, and any combinations thereof, wherein each mutation is numbered in reference to the sequence of SEQ ID NO: 80.

120. The method of claim 112, wherein the first gene editing protein and/or the second gene editing protein comprises a plurality of repeat sequences, and at least one of the repeat sequences comprises the amino acid sequence: LTPvQVVAIAwxyzα (SEQ ID NO: 22), wherein:
(a) v is Q, D or E,
(b) wis S or N,
(c) x is I, H, or N,
(d) y is D, A, I, N, H, K, S, G or null,
(e) z is GGRPALE (SEQ ID NO: 23), GGKQALE (SEQ ID NO: 24), GGKQALETVQRLLPVLCQDHG (SEQ ID NO: 25), GGKQALETVQRLLPVLCQAHG (SEQ ID NO: 26), GKQALETVQRLLPVLCQDHG (SEQ ID NO: 27), GKQALETVQRLLPVLCQAHG (SEQ ID NO: 28), GGKQALETVQRLLPVLCQD (SEQ ID NO: 19) or GGKQALETVQRLLPVLCQA (SEQ ID NO: 20), and
(f) α is any four consecutive amino acids.

121. The method of claim 120, wherein a comprises at least one, or two, or three of a glycine (G) residue, a histidine (H) residue, and/or an aspartic acid (D) residue.

122. A method for generating single-stranded DNA, the method comprising:
(a) obtaining a double-stranded DNA, wherein the double-stranded DNA comprises a strand comprising at least one phosphorothioate-containing nucleotide and a strand lacking a phosphorothioate-containing nucleotide; and
(b) contacting the double-stranded DNA with one or more nucleases which digest the strand lacking the phosphorothioate-containing nucleotide,
thereby generating the single-stranded DNA.

123. The method of claim 122, wherein the double-stranded DNA is synthesized by PCR using a 5′ primer lacking a phosphorothioate-containing nucleotide and a 5′ primer comprising one or more phosphorothioate-containing nucleotides.

124. The method of claim 123, wherein the method further comprises synthesizing the double-stranded DNA by PCR using a standard 5′ primer lacking the phosphorothioate-containing nucleotide and a 5′ primer comprising one or more phosphorothioate-containing nucleotides.

125. The method of claim 122, wherein the one or more nucleases comprise lambda exonuclease or T7 exonuclease.

126. The method of claim 122, wherein the one or more nucleases comprise lambda exonuclease and T7 exonuclease.

127. The method of claim 126, wherein the double-stranded DNA is contacted with lambda exonuclease before being contacted with T7 exonuclease.

128. The method of claim 122, wherein the strand comprising at least one phosphorothioate-containing nucleotide resists digestion by the one or more nucleases.

129. The method of claim 122, wherein the method reduces the amount of double-stranded contamination in a product comprising the single-stranded DNA by at least 95%.

130. The method of claim 122, wherein the double-stranded DNA comprises at least 50% GC base pairs.

131. The method of claim 122, wherein the single-stranded DNA is used as a repair template.


SEQUENCE LISTING (110 sequences)

SEQ ID NO: 1-15 - skipped (15 entries)

SEQ ID NO: 16 - protein, 34 aa, synthetic construct
  VARIANT 4: note = Q or D or E
  VARIANT 11: note = S or N
  VARIANT 12: note = I or H or N
  VARIANT 13: note = D or A or I or N or H or K or S or G or absent
  VARIANT 14..34: note = GGRPALE or GGKQALE or GGKQALETVQRLLPVLCQDHG or GGKQALETVQRLLPVLCQAHG or GKQALETVQRLLPVLCQDHG or GKQALETVQRLLPVLCQAHG or GGKQALETVQRLLPVLCQD or GGKQALETVQRLLPVLCQA
LTPXQVVAIAXXXXXXXXXXXXXXXXXXXXXXXX

SEQ ID NO: 17 - protein, 36 aa, synthetic construct
  VARIANT 4: note = Q or D or E
  VARIANT 11: note = S or N
  VARIANT 12: note = H or N or I
  VARIANT 13: note = D or A or I or N or G or H or K or S or G or absent
  VARIANT 14..32: note = GGKQALETVQRLLPVLCQD or GGKQALETVQRLLPVLCQA
LTPXQVVAIAXXXXXXXXXXXXXXXXXXXXXXGHGG

SEQ ID NO: 18 - protein, 36 aa, synthetic construct
  VARIANT 4: note = Q or D or E
  VARIANT 11: note = S or N
  VARIANT 12: note = H or N or I
  VARIANT 13: note = D or A or I or N or G or H or K or S or or absent
  VARIANT 14: note = GGKQALETVQRLLPVLCQD or GGKQALETVQRLLPVLCQA
LTPXQVVAIAXXXXXXXXXXXXXXXXXXXXXXGTHG

SEQ ID NO: 19 - protein, 19 aa, synthetic construct
GGKQALETVQRLLPVLCQD

SEQ ID NO: 20 - protein, 19 aa, synthetic construct
GGKQALETVQRLLPVLCQA

SEQ ID NO: 21 - protein, 4 aa, synthetic construct
GTHG

SEQ ID NO: 22 - protein, 38 aa, synthetic construct
  VARIANT 4: note = Q or D or E
  VARIANT 11: note = S or N
  VARIANT 12: note = I or H or N
  VARIANT 13: note = D or A or I or N or H or K or S or G or absent
  VARIANT 14..34: note = GGRPALE or GGKQALE or GGKQALETVQRLLPVLCQDHG or GGKQALETVQRLLPVLCQAHG or GKQALETVQRLLPVLCQDHG or GKQALETVQRLLPVLCQAHG or GGKQALETVQRLLPVLCQD or GGKQALETVQRLLPVLCQA
  VARIANT 35..38: note = Any amino acid
LTPXQVVAIAXXXXXXXXXXXXXXXXXXXXXXXXXXXX

SEQ ID NO: 23 - protein, 7 aa, synthetic construct
GGRPALE

SEQ ID NO: 24 - protein, 7 aa, synthetic construct
GGKQALE

SEQ ID NO: 25 - protein, 21 aa, synthetic construct
GGKQALETVQRLLPVLCQDHG

SEQ ID NO: 26 - protein, 21 aa, synthetic construct
GGKQALETVQRLLPVLCQAHG

SEQ ID NO: 27 - protein, 20 aa, synthetic construct
GKQALETVQRLLPVLCQDHG

SEQ ID NO: 28 - protein, 20 aa, synthetic construct
GKQALETVQRLLPVLCQAHG

SEQ ID NO: 29 - protein, 19 aa, synthetic construct
GGKQALETVQRLLPVLCQD

SEQ ID NO: 30 - protein, 36 aa, synthetic construct
  VARIANT 4: note = D or E
  VARIANT 11: note = S or N
  VARIANT 12: note = N or H or I
  VARIANT 13: note = Any amino acid or absent
  VARIANT 14..34: note = GGRPALE or GGKQALE or GGKQALETVQRLLPVLCQDHG or GGKQALETVQRLLPVLCQAHG or GKQALETVQRLLPVLCQDHG or GKQALETVQRLLPVLCQAHG
LTPXQVVAIAXXXXXXXXXXXXXXXXXXXXXXXXHG

SEQ ID NO: 31 - protein, 4 aa, synthetic construct
GHGG

SEQ ID NO: 32 - protein, 4 aa, synthetic construct
HGSG

SEQ ID NO: 33 - protein, 4 aa, synthetic construct
HGGG

SEQ ID NO: 34 - protein, 4 aa, synthetic construct
GGHD

SEQ ID NO: 35 - protein, 4 aa, synthetic construct
GAHD

SEQ ID NO: 36 - protein, 4 aa, synthetic construct
AHDG

SEQ ID NO: 37 - protein, 4 aa, synthetic construct
PHDG

SEQ ID NO: 38 - protein, 4 aa, synthetic construct
GPHD

SEQ ID NO: 39 - protein, 4 aa, synthetic construct
GHGP

SEQ ID NO: 40 - protein, 4 aa, synthetic construct
PHGG

SEQ ID NO: 41 - protein, 4 aa, synthetic construct
PHGP

SEQ ID NO: 42 - protein, 4 aa, synthetic construct
AHGA

SEQ ID NO: 43 - protein, 4 aa, synthetic construct
LHGA

SEQ ID NO: 44 - protein, 4 aa, synthetic construct
VHGA

SEQ ID NO: 45 - protein, 4 aa, synthetic construct
IVHG

SEQ ID NO: 46 - protein, 4 aa, synthetic construct
IHGM

SEQ ID NO: 47 - protein, 4 aa, synthetic construct
RHGD

SEQ ID NO: 48 - protein, 4 aa, synthetic construct
RDHG

SEQ ID NO: 49 - protein, 4 aa, synthetic construct
RHGE

SEQ ID NO: 50 - protein, 4 aa, synthetic construct
HRGE

SEQ ID NO: 51 - protein, 4 aa, synthetic construct
HRGD

SEQ ID NO: 52 - protein, 4 aa, synthetic construct
GPYE

SEQ ID NO: 53 - protein, 4 aa, synthetic construct
NHGG

SEQ ID NO: 54 - protein, 4 aa, synthetic construct
THGG

SEQ ID NO: 55 - protein, 36 aa, synthetic construct
  VARIANT 4: note = D or E
  VARIANT 12: note = S or N
  VARIANT 13: note = Any amino acid other than G
  VARIANT 14..34: note = GGRPALE or GGKQALE or GGKQALETVQRLLPVLCQDHG or GGKQALETVQRLLPVLCQAHG or GKQALETVQRLLPVLCQDHG or GKQALETVQRLLPVLCQAHG
LTPXQVVAIAIXXXXXXXXXXXXXXXXXXXXXXXHG

SEQ ID NO: 56 - protein, 4 aa, synthetic construct
GSGS

SEQ ID NO: 57 - protein, 4 aa, synthetic construct
GSGG

SEQ ID NO: 58 - protein, 4 aa, synthetic construct
GGGG

SEQ ID NO: 59 - protein, 4 aa, synthetic construct
GRGG

SEQ ID NO: 60 - protein, 4 aa, synthetic construct
GKGG

SEQ ID NO: 61 - DNA, 150 bp, synthetic construct
  source 1..150: mol_type = other DNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - DNA, 150 bp, synthetic construct
  source 1..150: mol_type = other DNA
aaaaaaaaaaaaaaaaaaaaaaaaaaaaaaaaaaaaaaagaaaaaaaaaaaaaaaaaaaaaaaaaaaaaaaaaaaaaaagaaaaaaaaaaaaaaaaaaaaaaaaaaaaaaaaaaaaaaagaaaaaaaaaaaaaaaaaaaaaaaaaaaaaa

SEQ ID NO: 63 - DNA, 150 bp, synthetic construct
  source 1..150: mol_type = other DNA
aaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaaaaaaaaaaagaaaaaaaaaa

SEQ ID NO: 64 - DNA, 150 bp, synthetic construct
  source 1..150: mol_type = other DNA
aaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaagaaaaaaaaag

SEQ ID NO: 65 - protein, 36 aa, synthetic construct
  VARIANT 12: note = I or H or N
  VARIANT 13: note = D or A or I or N or H or K or S or G or absent
LTPEQVVAIASXXGGKQALETVQRLLPVLCQAGHGG

SEQ ID NO: 66 - protein, 36 aa, synthetic construct
  VARIANT 4: note = D or E
  VARIANT 11: note = S or N
  VARIANT 14..34: note = GGRPALE or GGKQALE or GGKQALETVQRLLPVLCQDHG or GGKQALETVQRLLPVLCQAHG or GKQALETVQRLLPVLCQDHG or GKQALETVQRLLPVLCQAHG
LTPXQVVAIAXIAXXXXXXXXXXXXXXXXXXXXXHG

SEQ ID NO: 67 - protein, 12 aa, synthetic construct
  VARIANT 4: note = Q or D or E
  VARIANT 11: note = S or N
  VARIANT 12: note = S or T or Q
LTPXQVVAIAXX

SEQ ID NO: 68 - protein, 18 aa, synthetic construct
GKQALETVQRLLPVLCQA

SEQ ID NO: 69 - protein, 18 aa, synthetic construct
GKQALETVQRLLPVLCQD

SEQ ID NO: 70 - protein, 36 aa, synthetic construct; identical to SEQ ID NO: 17
  VARIANT 4: note = Q or D or E
  VARIANT 11: note = S or N
  VARIANT 12: note = Any amino acid other than G
  VARIANT 13: note = GGRPALE or GGKQALE or GGKQALETVQRLLPVLCQD or GGKQALETVQRLLPVLCQA or GKQALETVQRLLPVLCQD or GKQALETVQRLLPVLCQA

SEQ ID NO: 71 - protein, 36 aa, synthetic construct
  VARIANT 4: note = Q or D or E
  VARIANT 11: note = S or N
  VARIANT 14..32: note = GGRPALE or GGKQALE or GGKQALETVQRLLPVLCQD or GGKQALETVQRLLPVLCQA or GKQALETVQRLLPVLCQD or GKQALETVQRLLPVLCQA
LTPXQVVAIAXIAXXXXXXXXXXXXXXXXXXXGHGG

SEQ ID NO: 72 - protein, 17 aa, synthetic construct
  VARIANT 4: note = Q or D or E
  VARIANT 11: note = S or N
  VARIANT 12: note = S or T or Q
  VARIANT 13: note = D or A or I or N or H or K or S or and G
LTPXQVVAIAXXXGHGG

SEQ ID NO: 73 - protein, 16 aa, synthetic construct
DIVGVGKQWSGARALE

SEQ ID NO: 74 - protein, 16 aa, synthetic construct
DIVGVGKQKRGARALE

SEQ ID NO: 75 - protein, 20 aa, synthetic construct
DIVGVGGSKRGAGSGARALE

SEQ ID NO: 76 - protein, 360 aa, synthetic construct
MAGHLASDFAFSPPPGGGGDGPGGPEPGWVDPRTWLSFQGPPGGPGIGPGVGPGSEVWGIPPCPPPYEFCGGMAYCGPQVGVGLVPQGGLETSQPEGEAGVGVESNSDGASPEPCTVTPGAVKLEKEKLEQNPEESQDIKALQKELEQFAKLLKQKRITLGYTQADVGLTLGVLFGKVFSQTTICRFEALQLSFKNMCKLRPLLQKWVEEADNNENLQEICKAETLVQARKRKRTSIENRVRGNLENLFLQCPKPTLQQISHIAQQLGLEKDVVRVWFCNRRQKGKRSSSDYAQREDFEAAGSPFSGGPVSFPLAPGPHFGTPGYGSPHFTALYSSVPFPEGEAFPPVSVTTLGSPMHSN

SEQ ID NO: 77 - protein, 317 aa, synthetic construct
MYNMMETELKPPGPQQTSGGGGGNSTAAAAGGNQKNSPDRVKRPMNAFMVWSRGQRRKMAQENPKMHNSEISKRLGAEWKLLSETEKRPFIDEAKRLRALHMKEHPDYKYRPRRKTKTLMKKDKYTLPGGLLAPGGNSMASGVGVGAGLGAGVNQRMDSYAHMNGWSNGSYSMMQDQLGYPQHPGLNAHGAAQMQPMHRYDVSALQYNSMTSSQTYMNGSPTYSMSYSQQGTPGMALGSMGSVVKSEASSSPPVVTSSSHSRAPCQAGDLRDMISMYLPGAEVPEPAAPSRLHMSQHYQSGPVPGTAINGTLPLSHM

SEQ ID NO: 78 - protein, 477 aa, synthetic construct
QPPGESDMAVSDALLPSFSTFASGPAGREKTLRQAGAPNNRWREELSHMKRLPPVLPGRPYDLAAATVATDLESGGAGAACGGSNLAPLPRRETEEFNDLLDLDFILSNSLTHPPESVAATVSSSASASSSSSPSSSGPASAPSTCSFTYPIRAGNDPGVAPGGTGGGLLYGRESAPPPTAPFNLADINDVSPSGGFVAELLRPELDPVYIPPQQPQPPGGGLMGKFVLKASLSAPGSEYGSPSVISVSKGSPDGSHPVVVAPYNGGPPRTCPKIKQEAVSSCTHLGAGPPLSNGHRPAAHDFPLGRQLPSRTTPTLGLEEVLSSRDCHPALPLPPGFHPHPGPNYPSFLPDQMQPQVPPLHYQELMPPGSCMPEEPKPKRGRRSWPRKRTATHTCDYAGCGKTYTKSSHLKAHLRTHTGEKPYHCDWDGCGWKFARSDELTRHYRKHTGHRPFQCQKCDRAFSRSDHLALHMKRHF

SEQ ID NO: 79 - protein, 454 aa, synthetic construct
MDFFRVVENQQPPATMPLNVSFTNRNYDLDYDSVQPYFYCDEEENFYQQQQQSELQPPAPSEDIWKKFELLPTPPLSPSRRSGLCSPSYVAVTPFSLRGDNDGGGGSFSTADQLEMVTELLGGDMVNQSFICDPDDETFIKNIIIQDCMWSGFSAAAKLVSEKLASYQAARKDSGSPNPARGHSVCSTSSLYLQDLSAAASECIDPSVVFPYPLNDSSSPKSCASQDSSAFSPSSDSLLSSTESSPQGSPEPLVLHEETPPTTSSDSEEEQEDEEEIDVVSVEKRQAPGKRSESGSPSAGGHSKPPHSPLVLKRCHVSTHQHNYAAPPSTRKDYPAAKRVKLDSVRVLRQISNNRKCTSPRSSDTEENVKRRTHNVLERQRRNELKRSFFALRDQIPELENNEKAPKVVILKKATAYILSVQAEEQKLISEEDLLRKRREQLKHKLEQLRNSCA

SEQ ID NO: 80 - protein, 198 aa, synthetic construct
QLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 81 - protein, 198 aa, synthetic construct
QLVKSELEEKKSELRHKLKYVPHEYIELIEIARNSTQDRILEMKVMEFFMKVYGYRGKHLGGSRKPAGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 82 - protein, 198 aa, synthetic construct
QLVKSELEEKKSELRHKLKYVPHEYIELIEIARNSTQDRILEMKVMEFFMKVYGYRGKHLGGSRKPNGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 83 - protein, 198 aa, synthetic construct
QLVKSELEEKKSELRHKLKYVPHEYIELIEIARNSTQDRILEMKVMEFFMKVYGYRGKHLGGSRKPDGAIYTVGSPIDYGVIVATKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 84 - DNA, 597 bp, synthetic construct
  source 1..597: mol_type = other DNA
caactcgtgaagagtgaacttgaggagaaaaagtcggagctgcggcacaaattgaaatacgtaccgcatgaatacatcgaacttatcgaaattgctaggaactcgactcaagacagaatccttgagatgaaggtaatggagttctttatgaaggtttatggataccgagggaagcatctcggtggatcacgaaaacccgacggagcaatctatacggtggggagcccgattgattacggagtgatcgtcgacacgaaagcctacagcggtgggtacaatcttcccatcgggcaggcagatgagatgcaacgttatgtcgaagaaaatcagaccaggaacaaacacatcaatccaaatgagtggtggaaagtgtatccttcatcagtgaccgagtttaagtttttgtttgtctctgggcatttcaaaggcaactataaggcccagctcacacggttgaatcacattacgaactgcaatggtgcggttttgtccgtagaggaactgctcattggtggagaaatgatcaaagcgggaactctgacactggaagaagtcagacgcaagtttaacaatggcgagatcaatttccgctcataa

SEQ ID NO: 85 - DNA, 597 bp, synthetic construct
  source 1..597: mol_type = other DNA
caactcgtgaagagtgaacttgaggagaaaaagtcggagctgcggcacaaattgaaatacgtaccgcatgaatacatcgaacttatcgaaattgctaggaactcgactcaagacagaatccttgagatgaaggtaatggagttctttatgaaggtttatggataccgagggaagcatctcggtggatcacgaaaacccgccggagcaatctatacggtggggagcccgattgattacggagtgatcgtcgacacgaaagcctacagcggtgggtacaatcttcccatcgggcaggcagatgagatgcaacgttatgtcgaagaaaatcagaccaggaacaaacacatcaatccaaatgagtggtggaaagtgtatccttcatcagtgaccgagtttaagtttttgtttgtctctgggcatttcaaaggcaactataaggcccagctcacacggttgaatcacattacgaactgcaatggtgcggttttgtccgtagaggaactgctcattggtggagaaatgatcaaagcgggaactctgacactggaagaagtcagacgcaagtttaacaatggcgagatcaatttccgctcataa

SEQ ID NO: 86 - DNA, 597 bp, synthetic construct
  source 1..597: mol_type = other DNA
caactcgtgaagagtgaacttgaggagaaaaagtcggagctgcggcacaaattgaaatacgtaccgcatgaatacatcgaacttatcgaaattgctaggaactcgactcaagacagaatccttgagatgaaggtaatggagttctttatgaaggtttatggataccgagggaagcatctcggtggatcacgaaaacccaacggagcaatctatacggtggggagcccgattgattacggagtgatcgtcgacacgaaagcctacagcggtgggtacaatcttcccatcgggcaggcagatgagatgcaacgttatgtcgaagaaaatcagaccaggaacaaacacatcaatccaaatgagtggtggaaagtgtatccttcatcagtgaccgagtttaagtttttgtttgtctctgggcatttcaaaggcaactataaggcccagctcacacggttgaatcacattacgaactgcaatggtgcggttttgtccgtagaggaactgctcattggtggagaaatgatcaaagcgggaactctgacactggaagaagtcagacgcaagtttaacaatggcgagatcaatttccgctcataa

SEQ ID NO: 87 - DNA, 597 bp, synthetic construct
  source 1..597: mol_type = other DNA
caactcgtgaagagtgaacttgaggagaaaaagtcggagctgcggcacaaattgaaatacgtaccgcatgaatacatcgaacttatcgaaattgctaggaactcgactcaagacagaatccttgagatgaaggtaatggagttctttatgaaggtttatggataccgagggaagcatctcggtggatcacgaaaacccgacggagcaatctatacggtggggagcccgattgattacggagtgatcgtcgccacgaaagcctacagcggtgggtacaatcttcccatcgggcaggcagatgagatgcaacgttatgtcgaagaaaatcagaccaggaacaaacacatcaatccaaatgagtggtggaaagtgtatccttcatcagtgaccgagtttaagtttttgtttgtctctgggcatttcaaaggcaactataaggcccagctcacacggttgaatcacattacgaactgcaatggtgcggttttgtccgtagaggaactgctcattggtggagaaatgatcaaagcgggaactctgacactggaagaagtcagacgcaagtttaacaatggcgagatcaatttccgctcataa

SEQ ID NO: 88 - protein, 36 aa, synthetic construct
  VARIANT 4: note = Q or D or E
  VARIANT 11: note = S or N
  VARIANT 13: note = Any amino acid other than G
  VARIANT 14..32: note = GGRPALE or GGKQALE or GGKQALETVQRLLPVLCQD or GGKQALETVQRLLPVLCQA or GKQALETVQRLLPVLCQD or GKQALETVQRLLPVLCQA
LTPXQVVAIAXIXXXXXXXXXXXXXXXXXXXXGHGG

SEQ ID NO: 89 - protein, 4 aa, synthetic construct
KQWS

SEQ ID NO: 90 - protein, 8 aa, synthetic construct
GSKRGAGS

SEQ ID NO: 91 - DNA, 18 bp, synthetic construct
  source 1..18: mol_type = other DNA
aaaccggacggagcaatt

SEQ ID NO: 92 - DNA, 18 bp, synthetic construct
  source 1..18: mol_type = other DNA
atcgtggctactaagctt

SEQ ID NO: 93 - DNA, 18 bp, synthetic construct
  source 1..18: mol_type = other DNA
aaaccggccggaggaatt

SEQ ID NO: 94 - DNA, 18 bp, synthetic construct
  source 1..18: mol_type = other DNA
aaaccgaacggaggaatt

SEQ ID NO: 95 - DNA, 18 bp, synthetic construct
  source 1..18: mol_type = other DNA
atcgtggatactaagctt

SEQ ID NO: 96 - protein, 11 aa, synthetic construct
  VARIANT 4: note = E or Q
LTPXQVVAIAS

SEQ ID NO: 97 - protein, 36 aa, synthetic construct; identical to SEQ ID NO: 17
  VARIANT 4: note = Q or D or E
  VARIANT 11: note = S or N
  VARIANT 12: note = H or N or I
  VARIANT 13: note = Any amino acid or absent
  VARIANT 14..32: note = GGRPALE or GGKQALE or GGKQALETVQRLLPVLCQD or GGKQALETVQRLLPVLCQA or GKQALETVQRLLPVLCQD or GKQALETVQRLLPVLCQA

SEQ ID NO: 98 - protein, 36 aa, synthetic construct; identical to SEQ ID NO: 17
  VARIANT 4: note = Q or D or E
  VARIANT 11: note = S or N
  VARIANT 12: note = H or N or I
  VARIANT 13: note = D or A or I or N or H or K or S or G
  VARIANT 14..32: note = GGRPALE or GGKQALE or GGKQALETVQRLLPVLCQD or GGKQALETVQRLLPVLCQA or GKQALETVQRLLPVLCQD or GKQALETVQRLLPVLCQA

SEQ ID NO: 99 - protein, 36 aa, synthetic construct; identical to SEQ ID NO: 30
  VARIANT 4: note = D or E
  VARIANT 11: note = S or N
  VARIANT 12: note = N or H or I
  VARIANT 13: note = D or A or I or N or H or K or S or G
  VARIANT 14..34: note = GGRPALE or GGKQALE or GGKQALETVQRLLPVLCQDHG or GGKQALETVQRLLPVLCQAHG or GKQALETVQRLLPVLCQDHG or GKQALETVQRLLPVLCQAHG

SEQ ID NO: 100 - protein, 36 aa, synthetic construct; identical to SEQ ID NO: 17
  VARIANT 4: note = Q or D or E
  VARIANT 11: note = S or N
  VARIANT 12: note = S or T or Q
  VARIANT 13: note = Any amino acid or absent
  VARIANT 14..32: note = GGRPALE or GGKQALE or GGKQALETVQRLLPVLCQD or GGKQALETVQRLLPVLCQA or GKQALETVQRLLPVLCQD or GKQALETVQRLLPVLCQA

SEQ ID NO: 101 - protein, 36 aa, synthetic construct; identical to SEQ ID NO: 17
  VARIANT 4: note = Q or D or E
  VARIANT 11: note = S or N
  VARIANT 12: note = S or T or Q
  VARIANT 13: note = D or A or I or N or H or K or S or G
  VARIANT 14..32: note = GGRPALE or GGKQALE or GGKQALETVQRLLPVLCQD or GGKQALETVQRLLPVLCQA or GKQALETVQRLLPVLCQD or GKQALETVQRLLPVLCQA

SEQ ID NO: 102 - protein, 36 aa, synthetic construct; identical to SEQ ID NO: 30
  VARIANT 4: note = D or E
  VARIANT 11: note = S or N
  VARIANT 12: note = Any amino acid other than N or H or I
  VARIANT 13: note = Any amino acid or absent
  VARIANT 14..34: note = GGRPALE or GGKQALE or GGKQALETVQRLLPVLCQDHG or GGKQALETVQRLLPVLCQAHG or GKQALETVQRLLPVLCQDHG or GKQALETVQRLLPVLCQAHG

SEQ ID NO: 103 - protein, 12 aa, synthetic construct
  VARIANT 4: note = D or E
  VARIANT 11: note = S or N
  VARIANT 12: note = S or T or Q
LTPXQVVAIAXX

SEQ ID NO: 104 - protein, 34 aa, synthetic construct; identical to SEQ ID NO: 16
  VARIANT 4: note = D or E
  VARIANT 11: note = S or N
  VARIANT 12: note = S or T or Q
  VARIANT 13: note = D or A or I or N or H or K or S or G or absent
  VARIANT 14..34: note = GGRPALE or GGKQALE or GGKQALETVQRLLPVLCQDHG or GGKQALETVQRLLPVLCQAHG or GKQALETVQRLLPVLCQDHG or GKQALETVQRLLPVLCQAHG or GGKQALETVQRLLPVLCQD or GGKQALETVQRLLPVLCQA

SEQ ID NO: 105 - protein, 36 aa, synthetic construct; identical to SEQ ID NO: 17
  VARIANT 4: note = Q or D or E
  VARIANT 11: note = S or N
  VARIANT 12: note = Any amino acid other than N or H or I
  VARIANT 13: note = Any amino acid or absent
  VARIANT 14..32: note = GGRPALE or GGKQALE or GGKQALETVQRLLPVLCQD or GGKQALETVQRLLPVLCQA or GKQALETVQRLLPVLCQD or GKQALETVQRLLPVLCQA

SEQ ID NO: 106 - protein, 36 aa, synthetic construct; identical to SEQ ID NO: 30
  VARIANT 4: note = Q or D or E
  VARIANT 11: note = S or N
  VARIANT 12: note = Any amino acid other than N or H or I
  VARIANT 13: note = Any amino acid or absent
  VARIANT 14..34: note = GGRPALE or GGKQALE or GGKQALETVQRLLPVLCQDHG or GGKQALETVQRLLPVLCQAHG or GKQALETVQRLLPVLCQDHG or GKQALETVQRLLPVLCQAHG

SEQ ID NO: 107 - DNA, 82 bp, synthetic construct
  source 1..82: mol_type = other DNA
gccaccagcattctctcttccactcctgcagggccccatcggctttcctggagaacgcgggctgaagggcgaccgtggagac

SEQ ID NO: 108 - DNA, 82 bp, synthetic construct
  source 1..82: mol_type = other DNA
gccaccagcattctctcttccactcctgcggggccccatcggttttcggggaaagcggggggagaggggacaccggggaccc

SEQ ID NO: 109 - DNA, 82 bp, synthetic construct
  source 1..82: mol_type = other DNA
cccccccccacttttttttttcctcctgcagggccccatcggctttcctggagaacgcgggctgaagggcgaccgtggagac

SEQ ID NO: 110 - DNA, 82 bp, synthetic construct; identical to SEQ ID NO: 107
  source 1..82: mol_type = other DNA